(19) United States
(12) Patent Application Publication
(10) Pub. No.: US 2022/0153847 A1
(43) Pub. Date: May 19, 2022

(54) ANTI-HLA-DQ2.5 ANTIBODY

(71) Applicant: Chugai Seiyaku Kabushiki Kaisha, Tokyo (JP)

(72) Inventors: Yuu Okura, Singapore (SG); Noriyuki Takahashi, Singapore (SG); Takashi Tsushima, Singapore (SG); Zulkarnain Harfuddin, Singapore (SG)

(73) Assignee: Chugai Seiyaku Kabushiki Kaisha, Tokyo (JP)

(21) Appl. No.: 17/438,496

(22) PCT Filed: Apr. 1, 2020
(86) PCT No.: PCT/JP2020/014978

§ 371 Date: Sep. 13, 2021

(30) Foreign Application Priority Data
Apr. 1, 2019 (JP) 2019-070242

(51) Int. Cl. C07K 16/28

(52) U.S. Cl. CPC C07K 16/2833; C07K 2317/92; C07K 2317/31; C07K 2317/565

(57) ABSTRACT

The present invention provides anti-HLA-DQ2.5 antibodies. The anti-HLA-DQ2.5 antibodies of the invention have binding activity to complexes formed by HLA-DQ2.5 and a gluten peptide, but have substantially no binding activity to complexes formed by HL A-DQ2.5 and an irrelevant peptide. Furthermore, it was found that the antibodies of the invention have inhibitory effects on T cell activation.

### TECHNICAL FIELD

The present invention relates to anti-HLA-DQ2.5 antibodies

### BACKGROUND ART

Celiac (coeliac) disease is an autoimmune disorder in which the ingestion of gluten causes damage to the small intestine in genetically-sensitive patients (NPL 1 to 5). About 1% of the Western population, i.e., 8 million people in the United States and the European Union are thought to suffer from celiac disease; however, no remarkable therapeutic advances have been achieved since the disease was recognized in 1940s.

Human leukemia antigens (HLAs) belonging to Major Histocompatibility Complex (MHC) class II include HLA-DR, HLA-DP and HLA-DQ molecules such as the HLA-DQ2.5 isoform (hereinafter referred to as “HLA-DQ2.5”), which form heterodimers composed of alpha and beta chains on the cell surface. A majority (>90%) of the celiac disease patients have an HLA-DQ2.5 haplotype allele (NPL 6). The isoform is thought to have stronger affinity towards a gluten peptide. As with other isoforms, HLA-DQ2.5 presents processed antigens derived from exogenous sources to a T cell receptor (TCR) on T cells. As a result of digestion of gluten-rich food such as bread in celiac disease patients, immunogenic gluten peptides such as gliadin peptides are formed (NPL 2). The peptides are transported through the small intestine epithelium into lamina propria and deamidated by tissue transglutaminase such as transglutaminase 2 (TG2). The deamidated gliadin peptides are processed by antigen-presenting cells (APCs) which load them on HLA-DQ2.5. The loaded peptides are presented to HLA-DQ2.5-restricted T cells, and activate innate and adaptive immune responses. This causes inflammatory injury of the small intestinal mucosa and symptoms including various types of gastrointestinal disturbance, nutritional deficiencies, and systemic symptoms. It is reported that an anti-HLA DQ neutralizing antibody inhibits activation of T cells from celiac patients. (NPL7)

The currently practicable treatment of celiac disease is lifelong adherence to a gluten-free diet (GFD). However, in reality, it is difficult to completely eliminate gluten exposure even with GFD. The tolerable gluten dose for these patients is only about 10 to 50 mg/day (NPL 11). Cross contamination can widely occur in GFD production, and a trace amount of gluten can cause celiac disease symptoms even in patients with good compliance to GFD. In the presence of such a risk of unintentional gluten exposure, there is a need for adjunctive therapy to GFD.

### CITATION LIST

### Non Patent Literature

[NPL 1] N Engl J Med 2007; 357:1731-1743
[NPL 2] J Biomed Sci. 2012; 19(1): 88
[NPL 3] N Engl J Med 2003; 348:2517-2524
[NPL 4] Gut 2003; 52:960-965
[NPL 5] Dig Dis Sci 2004; 49:1479-1484
[NPL 6] Gastroenterology 2011; 141:610-620
[NPL 7] Gut 2005; 54:1217-1223
[NPL 8] Gastroenterology 2014; 146:1649-58
[NPL 9] Nutrients 2013 Oct. 5(10): 3975-3992
[NPL 10] J Clin Invest. 2007; 117(1):41-49
[NPL 11] Am J Clin Nutr 2007; 85: 160-6

### SUMMARY OF INVENTION

### Technical Problem

Under the above-mentioned circumstances with the need for adjunctive therapy, the present invention provides anti-HLA-DQ2.5 antibodies.

### Solution to Problem

The antigen-binding molecules, in particular, monospecific and multispecific (e.g., bispecific) antibodies of the present invention can bind to one or more complexes formed by HLA-DQ2.5 and a gluten peptide.

More specifically, the present invention provides the following.

[1] An antigen-binding molecule which has binding activity to at least one, two, three, four, five, six, seven, eight, nine, or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,

wherein the antigen-binding molecule has substantially no binding activity to either or both of a HLA-DQ2.5 positive PBMC B cell and a Ba/F3 cell that expresses HLA-DQ2.5.

[1-2] The antigen-binding molecule of [1], wherein the antigen-binding molecule which has binding activity to at least one, two, three, four, five, six, seven eight, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,

wherein the antigen-binding molecule has substantially no binding activity to either or both of a HLA-DQ2.5 positive PBMC B cell and a Ba/F3 cell that expresses HLA-DQ2.5.

[2] The antigen-binding molecule of [1], wherein the antigen-binding molecule has binding activity to at least one, two, three, or four or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; and complex formed by HLA-DQ2.5 and a 14 mer 1 peptide,

wherein the antigen-binding molecule has substantially no binding activity to either or both of a HLA-DQ2.5 positive PBMC B cell and a Ba/F3 cell that expresses HLA-DQ2.5.

[2-2] The antigen-binding molecule of [2], wherein the antigen-binding molecule has binding activity to at least one, two, three, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; and complex formed by HLA-DQ2.5 and a 14 mer 1 peptide,

wherein the antigen-binding molecule has substantially no binding activity to either or both of a HLA-DQ2.5 positive PBMC B cell and a Ba/F3 cell that expresses HLA-DQ2.5.

[3] The antigen-binding molecule of [1], wherein the antigen-binding molecule has binding activity to at least three, four, five, six, seven, eight, nine, or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,

wherein the antigen-binding molecule has substantially no binding activity to either or both of a HLA-DQ2.5 positive PBMC B cell and a Ba/F3 cell that expresses HLA-DQ2.5.

[3-2] The antigen-binding molecule of [3], wherein the antigen-binding molecule has binding activity to at least three, four, five, six, seven, eight, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,

wherein the antigen-binding molecule has substantially no binding activity to either or both of a HLA-DQ2.5 positive PBMC B cell and a Ba/F3 cell that expresses HLA-DQ2.5.

[4] An antigen-binding molecule which has binding activity to all of: complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide,

wherein the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLA-DQ2.5.

[4-2] The antigen-binding molecule of [4], wherein the antigen-binding molecule has binding activity to all of: complex formed by HLA-DQ2.5 and a BC hordein peptide; and complex formed by HLA-DQ2.5 and a gamma 1 gliadin peptide,

wherein the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLA-DQ2.5.

[5] The antigen-binding molecule of [4], wherein the antigen-binding molecule has binding activity to all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; and complex formed by HLA-DQ2.5 and a 26 mer gliadin,

wherein the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLA-DQ2.5.

[6] The antigen-binding molecule of [5], wherein the antigen-binding molecule has binding activity to a complex formed by HLA-DQ2.5 and an immune dominant peptide related to celiac disease.

[7] The antigen-binding molecule of [5], wherein the antigen-binding molecule has binding activity to all of: a complex formed by HLA-DQ2.5 and an immune dominant peptide related to celiac disease; complex formed by HLA-DQ2.5 and a 26 mer gliadin peptide; and complex formed by HLA-DQ2.5 and a 14 mer 1 peptide.

[8] The antigen-binding molecule of [5], wherein the antigen-binding molecule has binding activity to all of: complex formed by HLA-DQ2.5 and a 33mer gliadin peptide; complex formed by HLA-DQ2.5 and an alpha 1 gliadin peptide; complex formed by HLA-DQ2.5 and an alpha 2 gliadin peptide; complex formed by HLA-DQ2.5 and a gamma 1 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and an alpha 3 gliadin peptide; complex formed by HLA-DQ2.5 and an alpha 1b gliadin peptide; complex formed by HLA-DQ2.5 and a gamma 4b gliadin peptide; complex formed by HLA-DQ2.5 and an avenin 1 peptide; complex formed by HLA-DQ2.5 and an avenin 2 peptide; complex formed by HLA-DQ2.5 and a hordein 1 peptide; complex formed by HLA-DQ2.5 and a hordein 2 peptide; complex formed by HLA-DQ2.5 and a secalin 1 peptide; complex formed by HLA-DQ2.5 and a secalin 2 peptide; complex formed by HLA-DQ2.5 and a 14mer 1 peptide; and complex formed by HLA-DQ2.5 and a 26mer gliadin peptide.

[5-2] The antigen-binding molecule of [5], wherein the antigen-binding molecule has binding activity to all of: complex formed by HLA-DQ2.5 and a BC hordein peptide; complex formed by HLA-DQ2.5 and a gamma 1 gliadin peptide; and complex formed by HLA-DQ2.5 and a 26 mer gliadin,

wherein the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLA-DQ2.5.

[9] The antigen-binding molecule of any one of [1] to [8], wherein the antigen-binding molecule blocks the interaction between HLA-DQ2.5/gluten peptide complex and HLA-DQ2.5/gluten peptide-restricted CD4+ T cell.

[10] The antigen-binding molecule of any one of [1] or [9], wherein the antigen-binding molecule has substantially no binding activity to HLA-DQ8, HLA-DQ2.2, HLA-DQ7.5, HLA-DQ5.1, HLA-DQ6.3, HLADQ7.3, HLA-DR or HLA-DP.

[11] The antigen-binding molecule of any one of [1] to [10], wherein the antigen-binding molecule has enhanced binding activity to a complex formed by HLA-DQ2.5 and a gluten peptide.

[12] The antigen-binding molecule of any one of [1] to [11], wherein the antigen-binding molecule has stronger binding activity to at least one, two, three, four, five, six, seven, eight, nine, or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

[12-2] The antigen-binding molecule of [12] wherein the antigen-binding molecule of the invention has stronger binding activity to at least one, two, three, four, five, six, seven, eight, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

[13] An antigen-binding molecule which has binding activity to at least one, two, three, four, five, six, seven, eight, nine, or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,

wherein the antigen-binding molecule has substantially no binding activity to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and an HLADQ2.5 positive PBMC B cell,
wherein the antigen-binding molecule blocks the interaction between HLA-DQ2.5/gluten peptide complex and HLA-DQ2.5/gluten peptide-restricted CD4+ T cell.

[13-2] The antigen-binding molecule of [13], wherein the antigen-binding molecule of the invention has binding activity to at least one, two, three, four, five, six, seven, eight, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,

wherein the antigen-binding molecule has substantially no binding activity to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and an HLADQ2.5 positive PBMC B cell, where the antigen-binding molecule blocks the interaction between HLA-DQ2.5/gluten peptide complex and HLA-DQ2.5/gluten peptide-restricted CD4+ T cell. In this context, the gluten peptide is the peptide in the complex bound by any of the antigen-binding molecules described above.

[14] The antigen-binding molecule of any one of [1] to [13-2], which is any one of (1) to (5) below:

(1) an antigen-binding molecule comprising the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;
(2) an antigen-binding molecule comprising the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;
(3) an antigen-binding molecule comprising the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28;
(4) an antigen-binding molecule that binds to the same epitope bound by the antigen-binding molecule of any one of (1) to (3);
(5) an antigen-binding molecule that competes with the antigen-binding molecule of any one of (1) to (3) for binding to HLA-DQ2.5 or a complex formed by HLA-DQ2.5 and a gluten peptide.

[15] The antigen-binding molecule of any one of [1] to [14], wherein the antigen-binding molecule is a bispecific antigen-binding molecule.

[16] The antigen-binding molecule of [15], wherein the bispecific antigen-binding molecule is a bispecific antibody.

[17] An antigen-binding molecule that comprises at least two antigen-binding domains,

wherein either of the antigen-binding domains has binding activity to one or more complexes formed between HLA-DQ2.5 and an immune dominant peptide related to celiac disease,
wherein either of the antigen-binding domains has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

[18] An antigen-binding molecule that comprises at least two antigen-binding domains,

wherein either of the antigen-binding domains has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; and complex formed by HLA-DQ2.5 and a BC hordein peptide,
wherein either of the antigen-binding domains has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

[19] The antigen-binding molecule of [18], wherein either of the antigen-binding domains has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide.

[20] The antigen-binding molecule of [19], wherein either of the antigen-binding domains has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; and complex formed by HLA-DQ2.5 and a gamma 1 gliadin peptide.

[21] An antigen-binding molecule that comprises at least two antigen-binding domains], wherein either of the antigen-binding domains has binding activity to all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide,

wherein either of the antigen-binding domains has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

[22] The antigen-binding molecule of [21], wherein either of the antigen-binding domains has binding activity to all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide,

[23] An antigen-binding molecule that comprises a first antigen-binding domain and a second antigen-binding domain, wherein the first antigen-binding domain has binding activity to one or more complexes formed by HLA-DQ2.5 and a gluten peptide,

wherein the second antigen-binding domain has binding activity to one or more complexes formed by HLA-DQ2.5 and a gluten peptide, wherein at least one gluten peptide in the complexes bound by the first antigen-binding domain is different from at least one gluten peptide in the complexes bound by the second antigen-binding domain.

[24] The antigen-binding molecule of [23], wherein the antigen-binding molecule has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide.

[25] The antigen-binding molecule of [23], wherein the antigen-binding molecule has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; and complex formed by HLA-DQ2.5 and a gamma 1 gliadin peptide.

[26] The antigen-binding molecule of [23], wherein the antigen-binding molecule has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide,

wherein the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

[27] The antigen-binding molecule of [26], wherein the antigen-binding molecule has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; and complex formed by HLA-DQ2.5 and a gamma 1 gliadin peptide.

[28] An antigen-binding molecule that comprises a first antigen-binding domain which has binding activity to a complex formed by HLA-DQ2.5 and a first gluten peptide, and a second antigen-binding domain which has binding activity to a complex formed by HLA-DQ2.5 and a second gluten peptide,

wherein the antigen-binding molecule has binding activity to at least two or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide,
wherein the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

[29] The antigen-binding molecule of [28], wherein the antigen-binding molecule has binding activity to at least two or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide,

[30] An antigen-binding molecule that comprises a first antigen-binding domain and a second antigen-binding domain,

wherein the first antigen-binding domain has binding activity to at least one or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; and complex formed by HLA-DQ2.5 and a 33 mer gliadin peptide;
wherein the second antigen-binding domain has binding activity to at least one or more of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26mer gliadin peptide; complex formed by HLA-DQ2.5 and a 14mer 1 peptide; complex formed by HLA-DQ2.5 and a 33 mer gliadin peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide,
wherein the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

[31] The antigen-binding molecule of [30], wherein the second antigen-binding domain has binding activity to at least one or more of: complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and a 33 mer gliadin peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide.

[32]. The antigen-binding molecule of any one of [17] to [31], wherein the antigen-binding molecule blocks the interaction between HLA-DQ2.5/gluten peptide complex and HLADQ2.5/gluten peptide-restricted CD4+ T cell.

[33] The antigen-binding molecule of any one of [17] to [32], wherein the antigen-binding molecule has substantially no binding activity to HLA-DQ2.2, HLA-DQ7.5, HLA-DQ5.1, HLA-DQ6.3, HLADQ7.3, HLA-DR, or HLA-DP.

[34] The antigen-binding molecule of any one of [17] to [33], which has enhanced binding activity to a complex formed by HLA-DQ2.5 and a gluten peptide.

[35] The antigen-binding molecule of any one of [17] to [34], wherein the antigen-binding molecule has stronger binding activity to at least two, three, four, five, six, seven, eight, nine, ten, eleven, twelve, 13, 14, 15, 16, 17, 18, or all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

[36] The antigen-binding molecule of any one of [17] to [35], wherein the antigen-binding molecule has stronger binding activity to at least two, three, four, five, six, seven, eight, nine, ten, eleven, twelve, 13, 14, 15, 16, 17, or all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

[37] The antigen-binding molecule of any one of [17] to [36], which is any one of (1) to (5) below:

(1) an antigen-binding molecule comprising the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;
(2) an antigen-binding molecule comprising the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;
(3) an antigen-binding molecule comprising the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28;
(4) an antigen-binding molecule that binds to the same epitope bound by the antigen-binding molecule of any one of (1) to (3);
(5) an antigen-binding molecule that competes with the antigen-binding molecule of any one of (1) to (3) for binding to HLA-DQ2.5 or a complex formed by HLA-DQ2.5 and a gluten peptide.

[38] The antigen-binding molecule of any one of [17] to [37], wherein the antigen-binding molecule is a bispecific antigen-binding molecule.

[39] The antigen-binding molecule of [38], wherein the bispecific antigen-binding molecule is a bispecific antibody.

[40] The antigen-binding molecule of any one of [37] to [39], which is any one of (a) to (d) below:

(a) an antigen-binding molecule comprising (i) and (iii) below,
(b) an antigen-binding molecule comprising (ii) and (iii) below,
(c) an antigen-binding molecule that binds to the same epitope bound by the antigen-binding molecule of (a) or (b),
(d) an antigen-binding molecule that competes with the antigen-binding molecule of (a) or (b) for binding to HLA-DQ2.5 or a complex formed by HLA-DQ2.5 and a gluten peptide,
(i) the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;
(ii) the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;
(iii) the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28.

[40-1] The antigen-binding molecule of any one of [9], [13], and [32], wherein the gluten peptide(s) is/are one, two, three, four, five, six, seven, eight, or all of alpha 1 gliadin peptide, alpha 2 gliadin peptide, omega 1 gliadin peptide, omega 2 gliadin peptide, gamma 1 gliadin peptide, gamma 2 gliadin peptide, BC hordein peptide, alpha 1b gliadin peptide, and gamma 4a gliadin peptide.

[40-2] The antigen-binding molecule of [40-1], wherein the gluten peptides are alpha 1 gliadin peptide, alpha 2 glaidin peptide, omega 1 gliadin peptide, and alpha 1b gliadin peptide.

[40-3] The antigen-binding molecule of [40-1], wherein the gluten peptides are alpha 2 glaidin peptide, omega 2 gliadin peptide, gamma 1 gliadin peptide, gamma 2 gliadin peptide, BC hordein peptide, alpha 1b glaidin peptide, and gamma 4a gliadin peptide.

[40-4] The antigen-binding molecule of [40-1], wherein the gluten peptides are alpha 2 glaidin peptide, omega 2 gliadin peptide, gamma 1 gliadin peptide, and BC hordein peptide.

[40-5] The antigen-binding molecule of [40-1], wherein the gluten peptides are alpha 1 gliadin peptide, alpha 2 glaidin peptide, omega 1 gliadin peptide, omega 2 gliadin peptide, gamma 1 gliadin peptide, BC hordein peptide, alpha 1b glaidin peptide, gamma 4a gliadin peptide, and gamma 2 gliadin peptide.

[40-5a] The antigen-binding molecule of [40-1], wherein the gluten peptides are alpha 1 gliadin peptide, alpha 2 glaidin peptide, omega 1 gliadin peptide, omega 2 gliadin peptide, gamma 1 gliadin peptide, BC hordein peptide, alpha 1b glaidin peptide, and gamma 4a gliadin peptide.

[40-6] The antigen-binding molecule of [40-1], wherein the gluten peptides are alpha 1 gliadin peptide, alpha 2 glaidin peptide, omega 1 gliadin peptide, omega 2 gliadin peptide, gamma 1 gliadin peptide, BC hordein peptide, and alpha 1b glaidin peptide.

[41] A nucleic acid encoding the antigen-binding molecule of any one of [1] to [40-6].

[42] A vector into which the nucleic acid of [41] is introduced.

[43] A cell comprising the nucleic acid of [41] or the vector of [42].

[44] A method of producing an antigen-binding molecule by culturing the cell of [43].

[45] An antigen-binding molecule of any one of (1) to (5) below:

(1) an antigen-binding molecule comprising the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;
(2) an antigen-binding molecule comprising the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;
(3) an antigen-binding molecule comprising the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28;
(4) an antigen-binding molecule that binds to the same epitope bound by the antigen-binding molecule of any one of (1) to (3);
(5) an antigen-binding molecule that competes with the antigen-binding molecule of any one of (1) to (3) for binding to HLA-DQ2.5 or a complex formed by HLA-DQ2.5 and a gluten peptide.

[46] The antigen-binding molecule of [45], which is any one of (a) to (d) below:

(a) an antigen-binding molecule comprising (i) and (iii) below,
(b) an antigen-binding molecule comprising (ii) and (iii) below,
(c) an antigen-binding molecule that binds to the same epitope bound by the antigen-binding molecule of (a) or (b),
(d) an antigen-binding molecule that competes with the antigen-binding molecule of (a) or (b) for binding to HLA-DQ2.5 or a complex formed by HLA-DQ2.5 and a gluten peptide,
(i) the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20; (ii) the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24; (iii) the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28.

### BRIEF DESCRIPTION OF DRAWINGS

FIG. 1 shows analysis on binding of DQN0344xx//IC17 to complexes formed by HLA-DQ2.5 and gluten-derived peptides or irrelevant peptides. In the figure, “alpha”, “gamma”, and “omega” are abbreviated as “a”, “g”, and “w”. The same applies to other figures and other parts of the specification.

FIG. 2 shows analysis on binding of DQN0385ee//IC17 to complexes formed by HLA-DQ2.5 and gluten-derived peptides or irrelevant peptides.

FIG. 3 shows analysis on binding of DQN0429cc//IC17 to complexes formed by HLA-DQ2.5 and gluten-derived peptides or irrelevant peptides.

FIG. 4 shows analysis on binding of DQN0344xx//DQN0385ee to complexes formed by HLA-DQ2.5 and gluten-derived peptides or irrelevant peptides.

FIG. 5 shows analysis on binding of DQN0344xx//DQN0429cc to complexes formed by HLA-DQ2.5 and gluten-derived peptides or irrelevant peptides.

FIG. 6 shows analysis on binding of DQN0139bb//IC17 to complexes formed by HLA-DQ2.5 and gluten-derived peptides or irrelevant peptides.

FIG. 7 shows analysis on binding of DQN0344xx to complexes formed by HLA-DQ2.5 and gluten-derived peptides or irrelevant peptides.

FIG. 8 shows analysis on binding of DQN0385ee to complexes formed by HLA-DQ2.5 and gluten-derived peptides or irrelevant peptides.

FIG. 9 shows analysis on binding of DQN0429cc to complexes formed by HLA-DQ2.5 and gluten-derived peptides or irrelevant peptides.

FIG. 10 shows analysis on binding of DQN0139bb to complexes formed by HLA-DQ2.5 and gluten-derived peptides or irrelevant peptides.

FIG. 11 shows analysis on binding of IC17 to complexes formed by HLA-DQ2.5 and gluten-derived peptides or irrelevant peptides.

FIG. 12 shows analysis on binding of the antibodies to HLA-DQ5.1, HLA-DQ6.3, HLA-DR, and HLA-DP. The four bars, from left to right, show the results for HLA-DQ5.1, HLA-DQ6.3, HLA-DR, and HLA-DP, respectively.

FIG. 13 shows analysis on binding of the antibodies to HLA-DQ2.5-positive PBMC-B cells.

FIG. 14 shows analysis on binding of the antibodies to HLA-DQ2.5-positive PBMC-B cells.

FIG. 15 is a summary of the above results. Numeral data for FIG. 15 are shown in Table 4.

FIG. 16 is a summary of the above results. Numeral data for FIG. 16 are shown in Table 5.

FIG. 17 shows the neutralizing activity of the bivalent antibodies. For DQN0344xx, DQN0385ee, DQN0429cc, DQN0139bb, and IC17, the eight bars, from left to right, show the results for the antibody concentrations of 20, 5, 1.25, 0.3125, 0.078125, 0.019531, 0.004883, and 0.001221 microgram/mL, respectively.

FIG. 18 shows the neutralizing activity of the bispecific antibodies. For DQN0344xx//IC17, DQN0385ee//IC17, DQN0429cc//IC17, DQN0344xx//DQN0385ee, DQN0344xx//DQN0429cc, DQN0139bb//IC17, and IC17, the eight bars, from left to right, show the results for the antibody concentrations of 20, 5, 1.25, 0.3125, 0.078125, 0.019531, 0.004883, and 0.001221 microgram/mL, respectively.

FIG. 19 shows the ELISA result of the primary screening. The identified single hit (positive) B-cell clone could bind to IgG1 delta-GK and IgG4 delta-GK specifically but not to IgG1 delta-K and IgG4 delta-K. An anti-keyhole limpet hemocyanin (KLH) rabbit monoclonal antibody was used as an isotype control.

FIG. 20 shows the ELISA result of the secondary screening. The identified single hit (positive) B-cell clone could bind to IgG1 delta-GK and IgG4 delta-GK specifically but not to IgG1 delta-GK-amide and IgG4 delta-GK-amide. An anti-KLH rabbit monoclonal antibody was used as an isotype control.

FIG. 21 shows the ELISA result of the purified monoclonal antibody. YG55 could bind to IgG1 delta-GK and IgG4 delta-GK specifically but not to IgG1 delta-GK-amide and IgG4 delta-GK-amide. An anti-KLH rabbit monoclonal antibody was used as an isotype control.

FIG. 22 shows the inhibitory effect of DQN0344xx, DQN0385ee, DQN0429cc, DQN0139bb, DQN0344xx//DQN0385ee, and DQN0344xx//DQN0429cc on DQ2.5/alpha 1 gliadin dependent Jurkat T cell activation.

FIG. 23 shows the inhibitory effect of DQN0344xx, DQN0385ee, DQN0429cc, DQN0139bb, DQN0344xx//DQN0385ee, and DQN0344xx//DQN0429cc on DQ2.5/alpha 2 gliadin dependent Jurkat T cell activation.

FIG. 24 shows the inhibitory effect of DQN0344xx, DQN0385ee, DQN0429cc, DQN0139bb, DQN0344xx//DQN0385ee, and DQN0344xx//DQN0429cc on DQ2.5/omega 1 gliadin dependent Jurkat T cell activation.

FIG. 25 shows the inhibitory effect of DQN0344xx, DQN0385ee, DQN0429cc, DQN0139bb, DQN0344xx//DQN0385ee, and DQN0344xx//DQN0429cc on DQ2.5/omega 2 gliadin dependent Jurkat T cell activation.

FIG. 26 shows the inhibitory effect of DQN0344xx, DQN0385ee, DQN0429cc, DQN0139bb, DQN0344xx//DQN0385ee, and DQN0344xx//DQN0429cc on DQ2.5/gamma 1 gliadin dependent Jurkat T cell activation.

FIG. 27 shows the inhibitory effect of DQN0344xx, DQN0385ee, DQN0429cc, DQN0139bb, DQN0344xx//DQN0385ee, and DQN0344xx//DQN0429cc on DQ2.5/gamma 2 gliadin dependent Jurkat T cell activation.

FIG. 28 shows the inhibitory effect of DQN0344xx, DQN0385ee, DQN0429cc, DQN0139bb, DQN0344xx//DQN0385ee, and DQN0344xx//DQN0429cc on DQ2.5/BC hordein dependent Jurkat T cell activation.

FIG. 29 shows the inhibitory effect of DQN0344xx, DQN0385ee, DQN0429cc, DQN0139bb, DQN0344xx//DQN0385ee, and DQN0344xx//DQN0429cc on DQ2.5/alpha 1b gliadin dependent Jurkat T cell activation.

FIG. 30 shows the inhibitory effect of DQN0344xx, DQN0385ee, DQN0429cc, DQN0139bb, DQN0344xx//DQN0385ee, and DQN0344xx//DQN0429cc on DQ2.5/gamma 4a gliadin dependent Jurkat T cell activation.

FIG. 31 shows the inhibitory effect of DQN0344xx on DQ2.5/gluten peptides dependent Jurkat T cell activation.

FIG. 32 shows the inhibitory effect of DQN0385ee on DQ2.5/gluten peptides dependent Jurkat T cell activation.

FIG. 33 shows the inhibitory effect of DQN0429cc on DQ2.5/gluten peptides dependent Jurkat T cell activation.

FIG. 34 shows the inhibitory effect of DQN0344xx//DQN0385ee on DQ2.5/gluten peptides dependent Jurkat T cell activation.

FIG. 35 shows the inhibitory effect of DQN0344xx//DQN0429cc on DQ2.5/gluten peptides dependent Jurkat T cell activation.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “anti-HLA-DQ2.5 antibody” refers to an antibody that is capable of binding to HLA-DQ2.5 or one or more complexes formed by HLA-DQ2.5 and a gluten peptide with sufficient affinity such that the antibody is useful as a diagnostic and/or therapeutic agent in targeting HLA-DQ2.5. In one embodiment, the extent of binding of an anti-HLA-DQ2.5 antibody to an unrelated antigen is less than about 10% of the binding of the antibody to HLA-DQ2.5 or the HLA-DQ2.5/gluten peptide complex as measured, e.g., by a radioimmunoassay (RIA). In certain embodiments, an antibody which has “binding activity” to HLA-DQ2.5 or the HLA-DQ2.5/gluten peptide complex has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁸ M to 10⁻¹³M, e.g., from 10⁻⁹M to 10⁻¹³ M).

The term “antigen-binding molecule”, as used herein, refers to any molecule that comprises an antigen-binding site or any molecule that has binding activity to an antigen, and may further refers to molecules such as a peptide or protein having a length of about five amino acids or more. The peptide and protein are not limited to those derived from a living organism, and for example, they may be a polypeptide produced from an artificially designed sequence. They may also be any of a naturally-occurring polypeptide, synthetic polypeptide, recombinant polypeptide, and such. Scaffold molecules comprising known stable conformational structure such as alpha/beta barrel as scaffold, and in which part of the molecule is made into antigen-binding site, is also one embodiment of the antigen binding molecule described herein. In some embodiments, the “antigen-binding molecule” is an antibody. The terms “antigen-binding molecule” and “antibody” herein are used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. In some embodiments, the antibody is a multispecific antibody. In some embodiments, the multispecific antibody is a bispecific antibody.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Autoimmune disease” refers to a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, celiac disease, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobulinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

The term “celiac (coeliac) disease” refers to a hereditary autoimmune disease caused by damages in the small intestine upon ingestion of gluten contained in food. Symptoms of celiac disease include, but not limited to, gastrointestinal disturbance such as abdominal pain, diarrhea, and gastroesophageal reflux, vitamin deficiency, mineral deficiency, central nervous system (CNS) symptoms such as fatigue and anxiety depression, bone symptoms such as osteomalacia and osteoporosis, skin symptoms such as skin inflammation, blood symptoms such as anemia and lymphocytopenia, and other symptoms such as infertility, hypogonadism, and children's failure to thrive and short stature.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

Herein, the term “gluten” collectively refers to a composite of storage proteins called prolamins found in wheat and other related grains. In the gut lumen, gluten is degraded into so-called gluten peptides. Gluten peptides include, but are not limited to, gliadin from wheat, hordein from barley, and secalin from rye, and avenin from oat.

In celiac disease, gluten peptides are antigenic peptides recognized by T cells that cause the disease. Meanwhile, immune dominance is the phenomenon where immune response is mainly triggered by a relatively small number of antigenic peptides. Such antigenic peptides may be called “immune dominant peptides”. In celiac disease, such immune dominant peptides include, for example, alpha 1 gliadin and alpha 2 gliadin (both of which are included in the sequence of 33mer gliadin), and omega 1 gliadin, omega 2 gliadin, and BC hordein (five peptides in total) (Science Translational Medicine 21 Jul. 2010:Vol. 2, Issue 41, pp. 41ra51). Alternatively, the immune dominant peptides include alpha 1 gliadin, alpha 2 gliadin, omega 1 gliadin, omega 2 gliadin, BC hordein, gamma 1 gliadin, and gamma 2 gliadin (seven peptides in total), but are not limited thereto. Herein, such immune dominant peptides may be called “immune dominant peptides related to celiac disease”. As long as they are dominantly related to celiac disease, the types and total number of the peptides are not particularly limited.

The phrase “substantially no binding activity”, as used herein, refers to activity of an antibody to bind to an antigen of no interest at a level of binding that includes non-specific or background binding but does not include specific binding. In other words, such an antibody has “no specific/significant binding activity” towards the antigen of no interest. The specificity can be measured by any methods mentioned in this specification or known in the art. The above-mentioned level of non-specific or background binding may be zero, or may not be zero but near zero, or may be very low enough to be technically neglected by those skilled in the art. For example, when a skilled person cannot detect or observe any significant (or relatively strong) signal for binding between the antibody and the antigen of no interest in a suitable binding assay, it can be said that the antibody has “substantially no binding activity” or “no specific/significant binding activity” towards the antigen of no interest. Alternatively, “substantially no binding activity” or “no specific/significant binding activity” can be rephrased as “not specifically/significantly/substantially bind” (to the antigen of no interest). Sometimes, the phrase “no binding activity” has substantially the same meaning as the phrase “substantially no binding activity” or “no specific/significant binding activity” in the art.

Herein, “HLA-DR/DP” means “HLA-DR and HLA-DP” or “HLA-DR or HLA-DP”. These HLAs are MHC class II molecules encoded by the corresponding haplotype alleles on the MHC class II locus in human. “HLA-DQ” collectively refers to HLA-DQ isoforms including HLA-DQ2.5, HLA-DQ2.2, HLA-DQ7.5, HLA-DQ5.1, HLADQ6.3, HLA-DQ7.3, and HLA-DQ8. In the present invention, in addition to HLADQ2.5, HLA-DQ2.2, and HLA-DQ7.5, HLA-DQ molecules include, but are not limited to, HLA-DQ molecules of known subtypes (isoforms) such as HLA-DQ2.3, HLA-DQ4.3, HLA-DQ4.4, HLA-DQ5.1, HLA-DQ5.2, HLA-DQ5.3, HLA-DQ5.4, HLA-DQ6.1, HLA-DQ6.2, HLA-DQ6.3, HLA-DQ6.4, HLA-DQ6.9, HLA-DQ7.2, HLA-DQ7.3, HLA-DQ7.4, HLA-DQ7.5, HLA-DQ7.6, HLA-DQ8, HLA-DQ9.2, and HLA-DQ9.3. Similarly, “HLA-DR (DP)” refers to HLA-DR (DP) isoforms.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

In one embodiment, HVR residues comprise those identified in the specification.]] Unless otherwise indicated, HVR residues and other residues in the variable domain (e.g., FR residues) are numbered herein according to Kabat et al., supra.

An “immunoconjugate” is an antibody conjugated to one or more heterologous molecule(s).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

In the present invention, a CLIP peptide (for example, SEQ ID NO: 45) may be used together with a suitable HLA-DQ molecule such as HLA-DQ2.5, HLA-DQ2.2, and HLA-DQ7.5 when evaluating the binding of the anti-HLA-DQ2.5 antibodies to these HLA-DQ molecules. Meanwhile, for HLA-DQ5.1, a DBY peptide (for example, SEQ ID NO: 44) may be used for this purpose. This peptide is a portion of the DBY protein which is an HLA-DQ5-restricted histocompatibility antigen.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-HLA-DQ2.5 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa and lambda, based on the amino acid sequence of its constant domain.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “HLA-DQ2.5,” as used herein, refers to any native HLA-DQ2.5 from any vertebrate source, including mammals such as primates (e.g. humans) and rodents (e.g., mice and rats), unless otherwise indicated. The term encompasses “full-length” unprocessed HLA-DQ2.5 as well as any form of HLA-DQ2.5 that results from processing in the cell. The term also encompasses naturally occurring variants of HLADQ2.5, e.g., splice variants or allelic variants. The amino acid sequence of exemplary HLA-DQ2.5 is publicly available in Research Collaboratory for Structural Bioinformatics (RCSB) Protein Data Bank (PDB) accession code 4OZG.

Herein, “TCR” means “T-cell receptor” which is a membrane protein located on the surface of T cells (such as HLA-DQ2.5-restricted CD4+ T cells), and recognizes an antigen fragment (such as a gluten peptide) presented on MHC molecules including HLA-DQ2.5.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions

In one aspect, the invention is based, in part, on the binding of an anti-HLA-DQ2.5 antibody to HLA-DQ2.5 that presents a gluten peptide to T cells. In certain embodiments, antibodies that bind to HLA-DQ2.5 are provided.

A. Exemplary Anti-HLA-D02.5 Antigen-Binding Molecules/Antibodies

In one aspect, the invention provides isolated antigen-binding molecules or antibodies that has binding activity to HLA-DQ2.5 or one or more complexes formed by HLA-DQ2.5 and a gluten peptide. In certain embodiments, the anti-HLA-DQ2.5 antibody (“the antibody”) has the functions/characteristics below.

The antibody has binding activity to HLA-DQ2.5 or the HLA-DQ2.5/gluten peptide complex. In other words, the antibody binds to HLA-DQ2.5 or the HLA-DQ2.5/gluten peptide complex. More preferably, the antibody has specific binding activity to HLADQ2.5 or the HLA-DQ2.5/gluten peptide complex. That is, the antibody specifically binds to HLA-DQ2.5 or the HLA-DQ2.5/gluten peptide complex.

The antibody has substantially no binding activity to an antigen of no interest, such as HLA-DQ2.2/DQ5.1/DQ6.3/DQ7.3/DQ7.5/DQ8/DR/DP, i.e., the antibody does not substantially bind to the antigen of no interest. For example, the antibody has no specific binding activity to HLA-DR/DP or no significant binding activity to HLA-DR/DP. That is, the antibody does not specifically bind to HLA-DR/DP or significantly bind to HLA-DR/DP. Similarly, the antibody has substantially no binding activity to an HLA-DQ molecule such as HLA-DQ2.2, HLA-DQ7.5, HLA-DQ8, HLA-DQ5.1, HLA-DQ6.3, and HLA-DQ7.3, i.e., the antibody does not substantially bind to an HLA-DQ molecule such as HLA-DQ2.2, HLA-DQ7.5, HLA-DQ8, HLADQ5.1, HLA-DQ6.3, and HLA-DQ7.3. In other words, the antibody has no specific/significant binding activity to an HLA-DQ molecule such as HLA-DQ2.2, HLADQ7.5, HLA-DQ8, HLA-DQ5.1, HLA-DQ6.3, and HLA-DQ7.3. That is, the antibody does not specifically/significantly bind to an HLA-DQ molecule such as HLA-DQ2.2, HLA-DQ7.5, HLA-DQ8, HLA-DQ5.1, HLA-DQ6.3, and HLA-DQ7.3. To prevent any substantial inhibitory effects on these non-target MHC class II molecules, and to improve antibody PK for the celiac disease patients who have HLA-DQ2.5, these characteristics are preferable.

*The feature of the “substantially no binding activity” can be defined, for example, as described in the FACS results described herein. The antibody having “substantially no binding activity” to a specific antigen has an MFI (Mean Fluorescence Intensity) value that is 250% or less, preferably 200% or less, more preferably 150% or less of the MFI value of the negative control (e.g., herein, “IC17” in Table 4, and “IC17 bivalent” in Table 5) under the measurement conditions described herein.

In an aspect, for a bivalent antibody, the antibody having “substantially no binding activity” to a specific antigen has an MFI value that is 5% or less, preferably 4% or less, more preferably 3% or less, still more preferably 2% or less, yet more preferably 1% or less when taking a MFI value of the IC17 as 0% and a MFI value of the DQN0139bb (WO2018/155692) as 100% under the measurement conditions described herein.

In an aspect, for a bispecific antibody, the antibody having “substantially no binding activity” to a specific antigen has an MFI value that is 2% or less, more preferably 1% or less when taking a MFI value of the IC17 bivalent antibody as 0% and a MFI value of the DQN0139bb//IC17 as 100% under the measurement conditions described herein.

The antibody has binding activity to HLA-DQ2.5 that is in complex with a gluten peptide described herein. Herein, a complex formed between an HLA-DQ2.5 molecule and a gluten peptide is referred to as “a complex formed by HLA-DQ2.5 and a gluten peptide”, “an HLA-DQ2.5/gluten peptide complex”, or “HLA-DQ2.5/gluten peptide”. It may also be rephrased as, for example, “HLA-DQ2.5 loaded with a gluten peptide”, “gluten peptide-loaded HLA-DQ2.5”, “HLA-DQ2.5 bound by a gluten peptide”, “HLADQ2.5 in the form of a complex with a gluten peptide”, and “a complex of HLADQ2.5 and a gluten peptide”. The above wording (e.g., “a complex formed by HLADQ2.5 and . . . [peptide]”) also apply to peptides such as 33mer gliadin peptide, 26mer gliadin peptide, 14 mer 1 peptide, alpha 1 gliadin peptide, alpha 1b gliadin peptide, alpha 2 gliadin peptide, alpha 3 gliadin peptide, gamma 1 gliadin peptide, gamma 2 gliadin peptide, gamma 4b gliadin peptide, omega 1 gliadin peptide, omega 2 gliadin peptide, secalin 1 peptide, secalin 2 peptide, salmonella peptide, Mycobacterium bovis peptide, Hepatitis B virus peptide, BC hordein peptide, thyroperoxidase peptide, avenin 1 peptide, avenin 2 peptide, avenin 3 peptide, hordein 1 peptide, hordein 2 peptide, etc.

The gluten peptide is preferably a gliadin peptide. The gliadin peptide is preferably a 33mer gliadin peptide, a 26mer gliadin peptide, a 14 mer 1 peptide, an alpha 1 gliadin peptide, an alpha 1b gliadin peptide, an alpha 2 gliadin peptide, an alpha 3 gliadin peptide, a gamma 1 gliadin peptide, a gamma 2 gliadin peptide, a gamma 4b gliadin peptide, an omega 1 gliadin peptide or an omega 2 gliadin peptide.
In other aspects, the gluten peptide is preferably selected from the group consisting of: a BC hordein peptide, an avenin 1 peptide, an avenin 2 peptide, an avenin 3 peptide, a hordein 1 peptide, a hordein 2 peptide, a secalin 1 peptide, a secalin 2 peptide. Meanwhile, herein, “irrelevant” peptides include those that have been reported to be able to be presented on HLA-DQ2.5 but are irrelevant to the present invention, i.e., those which are not the above-mentioned gluten peptides of interest. For example, the irrelevant peptides include, but are not limited to, a CLIP peptide, a Hepatitis B virus (HBV) peptide, a salmonella peptide, a thyroperoxidase (TPO) peptide, Mycobacterium bovis peptide, etc.
To prevent any substantial inhibitory effects on these non-target MHC class II molecules and HLA-DQ2.5 in the form of a complex with irrelevant peptide, and to improve antibody PK for the celiac disease patients, these characteristics are preferable.
*The feature of the “binding activity” can be defined, for example, as described in the FACS results described herein. The antibody having “binding activity” to a specific antigen has an MFI (Mean Fluorescence Intensity) value that is 300% or above, preferably 500% or above, more preferably 1000% or above of the MFI value of the negative control (e.g., herein, “IC17” in Table 4, and “IC17 bivalent” in Table 5) under the measurement conditions described herein.

In an aspect, for a bivalent antibody, the antibody having “binding activity” to a specific antigen has an MFI value that is 7.5% or above, preferably 10% or above, more preferably 20% or above when taking a MFI value of the IC17 as 0% and a MFI value of the DQN0139bb as 100% under the measurement conditions described herein.

In an aspect, for a bispecific antibody, the antibody having “binding activity” to a specific antigen has an MFI value that is 3% or above, preferably 6% or above, preferably 10% or above, more preferably 20% or above when taking a MFI value of the IC17 bivalent antibody as 0% and a MFI value of the DQN0139bb//IC17 as 100% under the measurement conditions described herein.

When particularly referring to the specificity of binding, “binding activity” can be rephrased as “specific binding activity”.

*Anti-HLA-DQ2.5 antibodies of the invention have a dissociation constant (Kd) of 5×10⁻⁷ M or less, preferably 4×10⁻⁷ M or less, preferably 3×10⁻⁷ M or less, preferably 2×10⁻⁷ M or less, preferably 1×10⁻⁷ M or less, preferably 9×10⁻⁸ M or less, preferably 8×10⁻⁸ M or less, preferably 7×10⁻⁸ M or less, preferably 6×10⁻⁸ M or less, preferably 5×10⁻⁸ M or less, preferably 4×10⁻⁸ M or less, preferably 3×10⁻⁸ M or less, preferably 2×10⁻⁸ M or less, preferably 1×10⁻⁸ M or less, preferably 9×10⁻⁹ M or less, preferably 8×10⁻⁹ M or less, preferably 7×10⁻⁹ M or less, preferably 6×10 9 M or less, preferably 5×10⁻⁹ M or less, preferably 4×10⁻⁹ M or less, preferably 3×10⁻⁹ M or less, preferably 2×10⁻⁹ M or less, for binding to one or more complexes formed by HLA-DQ2.5 and a gluten peptide described herein.

In some embodiments, the antigen-binding molecules/domains of the invention have binding activity to (at least) one, two, three, four, five, six, seven, eight, nine, ten, eleven, twelve, 13, 14, 15, 16, 17, 18, 19, or all of the complexes (1) to (20) below:

(1) complex formed by HLA-DQ2.5 and a 33mer gliadin peptide;

(2) complex formed by HLA-DQ2.5 and an alpha 1 gliadin peptide;

(3) complex formed by HLA-DQ2.5 and an alpha 2 gliadin peptide;

(4) complex formed by HLA-DQ2.5 and a gamma 1 gliadin peptide;

(5) complex formed by HLA-DQ2.5 and a gamma 2 gliadin peptide;

(6) complex formed by HLA-DQ2.5 and an omega 1 gliadin peptide;

(7) complex formed by HLA-DQ2.5 and an omega 2 gliadin peptide;

(8) complex formed by HLA-DQ2.5 and a BC hordein peptide;

(9) complex formed by HLA-DQ2.5 and an alpha 3 gliadin peptide;

(10) complex formed by HLA-DQ2.5 and an alpha 1b gliadin peptide;

(11) complex formed by HLA-DQ2.5 and a gamma 4b gliadin peptide;

(12) complex formed by HLA-DQ2.5 and an avenin 1 peptide;

(13) complex formed by HLA-DQ2.5 and an avenin 2 peptide;

(14) complex formed by HLA-DQ2.5 and an avenin 3 peptide;

(15) complex formed by HLA-DQ2.5 and a hordein 1 peptide;

(16) complex formed by HLA-DQ2.5 and a hordein 2 peptide;

(17) complex formed by HLA-DQ2.5 and a secalin 1 peptide;

(18) complex formed by HLA-DQ2.5 and a secalin 2 peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have binding activity to (at least) one, two, three, four, five, six, seven, eight, nine, or all of the complexes below:

(1) complex formed by HLA-DQ2.5 and a 33mer gliadin peptide;

(2) complex formed by HLA-DQ2.5 and an alpha 1 gliadin peptide;

(3) complex formed by HLA-DQ2.5 and an alpha 2 gliadin peptide;

(4) complex formed by HLA-DQ2.5 and a gamma 1 gliadin peptide;

(5) complex formed by HLA-DQ2.5 and a gamma 2 gliadin peptide;

(6) complex formed by HLA-DQ2.5 and an omega 1 gliadin peptide;

(7) complex formed by HLA-DQ2.5 and an omega 2 gliadin peptide;

(8) complex formed by HLA-DQ2.5 and a BC hordein peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have binding activity to one, two, three, four, or all of the complexes below:

(4) complex formed by HLA-DQ2.5 and a gamma 1 gliadin peptide;

(5) complex formed by HLA-DQ2.5 and a gamma 2 gliadin peptide;

(8) complex formed by HLA-DQ2.5 and a BC hordein peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have binding activity to (at least) one, two, three, four, five, six, seven, eight, nine, ten, eleven, or all of the complexes below:

(1) complex formed by HLA-DQ2.5 and a 33mer gliadin peptide;

(2) complex formed by HLA-DQ2.5 and an alpha 1 gliadin peptide;

(3) complex formed by HLA-DQ2.5 and an alpha 2 gliadin peptide;

(6) complex formed by HLA-DQ2.5 and an omega 1 gliadin peptide;

(9) complex formed by HLA-DQ2.5 and an alpha 3 gliadin peptide;

(10) complex formed by HLA-DQ2.5 and an alpha 1b gliadin peptide;

(12) complex formed by HLA-DQ2.5 and an avenin 1 peptide;

(13) complex formed by HLA-DQ2.5 and an avenin 2 peptide;

(14) complex formed by HLA-DQ2.5 and an avenin 3 peptide;

(15) complex formed by HLA-DQ2.5 and a hordein 1 peptide;

(17) complex formed by HLA-DQ2.5 and a secalin 1 peptide; and

(18) complex formed by HLA-DQ2.5 and a secalin 2 peptide.

In some embodiments, the antigen-binding molecules/domains of the invention have binding activity to (at least) one, two, three, four, five, six, seven, eight, nine, ten, eleven, twelve, 13, 14, 15, 16, 17, 18, or all of the complexes below:

(1) complex formed by HLA-DQ2.5 and a 33mer gliadin peptide;

(2) complex formed by HLA-DQ2.5 and an alpha 1 gliadin peptide;

(3) complex formed by HLA-DQ2.5 and an alpha 2 gliadin peptide;

(4) complex formed by HLA-DQ2.5 and a gamma 1 gliadin peptide;

(5) complex formed by HLA-DQ2.5 and a gamma 2 gliadin peptide;

(6) complex formed by HLA-DQ2.5 and an omega 1 gliadin peptide;

(7) complex formed by HLA-DQ2.5 and an omega 2 gliadin peptide;

(8) complex formed by HLA-DQ2.5 and a BC hordein peptide;

(9) complex formed by HLA-DQ2.5 and an alpha 3 gliadin peptide;

(10) complex formed by HLA-DQ2.5 and an alpha 1b gliadin peptide;

(11) complex formed by HLA-DQ2.5 and a gamma 4b gliadin peptide;

(12) complex formed by HLA-DQ2.5 and an avenin 1 peptide;

(13) complex formed by HLA-DQ2.5 and an avenin 2 peptide;

(15) complex formed by HLA-DQ2.5 and a hordein 1 peptide;

(16) complex formed by HLA-DQ2.5 and a hordein 2 peptide;

(17) complex formed by HLA-DQ2.5 and a secalin 1 peptide;

(18) complex formed by HLA-DQ2.5 and a secalin 2 peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have binding activity to (at least) one, two, three, four, five, six, seven, eight, nine, ten, eleven, twelve, 13, 14, 15, 16, 17, or all of the complexes below:

(1) complex formed by HLA-DQ2.5 and a 33mer gliadin peptide;

(2) complex formed by HLA-DQ2.5 and an alpha 1 gliadin peptide;

(3) complex formed by HLA-DQ2.5 and an alpha 2 gliadin peptide;

(4) complex formed by HLA-DQ2.5 and a gamma 1 gliadin peptide;

(6) complex formed by HLA-DQ2.5 and an omega 1 gliadin peptide;

(7) complex formed by HLA-DQ2.5 and an omega 2 gliadin peptide;

(8) complex formed by HLA-DQ2.5 and a BC hordein peptide;

(9) complex formed by HLA-DQ2.5 and an alpha 3 gliadin peptide;

(10) complex formed by HLA-DQ2.5 and an alpha 1b gliadin peptide;

(11) complex formed by HLA-DQ2.5 and a gamma 4b gliadin peptide;

(12) complex formed by HLA-DQ2.5 and an avenin 1 peptide;

(13) complex formed by HLA-DQ2.5 and an avenin 2 peptide;

(15) complex formed by HLA-DQ2.5 and a hordein 1 peptide;

(16) complex formed by HLA-DQ2.5 and a hordein 2 peptide;

(17) complex formed by HLA-DQ2.5 and a secalin 1 peptide;

(18) complex formed by HLA-DQ2.5 and a secalin 2 peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have binding activity to (at least) one, two, three, four, five, six, seven, eight, nine, ten, eleven, twelve, 13, or all of the complexes below:

(1) complex formed by HLA-DQ2.5 and a 33mer gliadin peptide;

(2) complex formed by HLA-DQ2.5 and an alpha 1 gliadin peptide;

(3) complex formed by HLA-DQ2.5 and an alpha 2 gliadin peptide;

(4) complex formed by HLA-DQ2.5 and a gamma 1 gliadin peptide;

(6) complex formed by HLA-DQ2.5 and an omega 1 gliadin peptide;

(7) complex formed by HLA-DQ2.5 and an omega 2 gliadin peptide;

(8) complex formed by HLA-DQ2.5 and a BC hordein peptide;

(10) complex formed by HLA-DQ2.5 and an alpha 1b gliadin peptide;

(11) complex formed by HLA-DQ2.5 and a gamma 4b gliadin peptide;

(16) complex formed by HLA-DQ2.5 and a hordein 2 peptide;

(17) complex formed by HLA-DQ2.5 and a secalin 1 peptide;

(18) complex formed by HLA-DQ2.5 and a secalin 2 peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have binding activity to (at least) one, two, three, four, five, six, seven, eight, nine, ten, eleven, twelve, 13, 14, 15, 16, 17, 18, or all of the complexes below:

(1) complex formed by HLA-DQ2.5 and a 33mer gliadin peptide;

(2) complex formed by HLA-DQ2.5 and an alpha 1 gliadin peptide;

(3) complex formed by HLA-DQ2.5 and an alpha 2 gliadin peptide;

(4) complex formed by HLA-DQ2.5 and a gamma 1 gliadin peptide;

(6) complex formed by HLA-DQ2.5 and an omega 1 gliadin peptide;

(7) complex formed by HLA-DQ2.5 and an omega 2 gliadin peptide;

(8) complex formed by HLA-DQ2.5 and a BC hordein peptide;

(9) complex formed by HLA-DQ2.5 and an alpha 3 gliadin peptide;

(10) complex formed by HLA-DQ2.5 and an alpha 1b gliadin peptide;

(11) complex formed by HLA-DQ2.5 and a gamma 4b gliadin peptide;

(12) complex formed by HLA-DQ2.5 and an avenin 1 peptide;

(13) complex formed by HLA-DQ2.5 and an avenin 2 peptide;

(14) complex formed by HLA-DQ2.5 and an avenin 3 peptide;

(15) complex formed by HLA-DQ2.5 and a hordein 1 peptide;

(16) complex formed by HLA-DQ2.5 and a hordein 2 peptide;

(17) complex formed by HLA-DQ2.5 and a secalin 1 peptide;

(18) complex formed by HLA-DQ2.5 and a secalin 2 peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have binding activity to (at least) one, two, three, four, five, six, seven, eight, nine, ten, eleven, twelve, 13, or all of the complexes below:

(1) complex formed by HLA-DQ2.5 and a 33mer gliadin peptide;

(3) complex formed by HLA-DQ2.5 and an alpha 2 gliadin peptide;

(4) complex formed by HLA-DQ2.5 and a gamma 1 gliadin peptide;

(6) complex formed by HLA-DQ2.5 and an omega 1 gliadin peptide;

(7) complex formed by HLA-DQ2.5 and an omega 2 gliadin peptide;

(8) complex formed by HLA-DQ2.5 and a BC hordein peptide;

(9) complex formed by HLA-DQ2.5 and an alpha 3 gliadin peptide;

(10) complex formed by HLA-DQ2.5 and an alpha 1b gliadin peptide;

(11) complex formed by HLA-DQ2.5 and a gamma 4b gliadin peptide;

(16) complex formed by HLA-DQ2.5 and a hordein 2 peptide;

(17) complex formed by HLA-DQ2.5 and a secalin 1 peptide;

(18) complex formed by HLA-DQ2.5 and a secalin 2 peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have substantially no binding activity to (at least) one, two, three, four, five, six, seven, eight, nine, ten, eleven, twelve, 13, 14, 15, 16, 17, 18, or 19 of the complexes (1) to (20) below:

(1) complex formed by HLA-DQ2.5 and a 33mer gliadin peptide;

(2) complex formed by HLA-DQ2.5 and an alpha 1 gliadin peptide;

(3) complex formed by HLA-DQ2.5 and an alpha 2 gliadin peptide;

(4) complex formed by HLA-DQ2.5 and a gamma 1 gliadin peptide;

(5) complex formed by HLA-DQ2.5 and a gamma 2 gliadin peptide;

(6) complex formed by HLA-DQ2.5 and an omega 1 gliadin peptide;

(7) complex formed by HLA-DQ2.5 and an omega 2 gliadin peptide;

(8) complex formed by HLA-DQ2.5 and a BC hordein peptide;

(9) complex formed by HLA-DQ2.5 and an alpha 3 gliadin peptide;

(10) complex formed by HLA-DQ2.5 and an alpha 1b gliadin peptide;

(11) complex formed by HLA-DQ2.5 and a gamma 4b gliadin peptide;

(12) complex formed by HLA-DQ2.5 and an avenin 1 peptide;

(13) complex formed by HLA-DQ2.5 and an avenin 2 peptide;

(14) complex formed by HLA-DQ2.5 and an avenin 3 peptide;

(15) complex formed by HLA-DQ2.5 and a hordein 1 peptide;

(16) complex formed by HLA-DQ2.5 and a hordein 2 peptide;

(17) complex formed by HLA-DQ2.5 and a secalin 1 peptide;

(18) complex formed by HLA-DQ2.5 and a secalin 2 peptide;

(19) complex formed by HLA-DQ2.5 and a 14mer 1 peptide; and

(20) complex formed by HLA-DQ2.5 and a 26mer gliadin peptide.

In some embodiments, the antigen-binding molecules/domains of the invention have substantially no binding activity to (at least) one, two, three, four, five, six, or all of (a) to (g) below:

(a) complex formed by HLA-DQ2.5 and a CLIP peptide;

(b) complex formed by HLA-DQ2.5 and a Hepatitis B virus (HBV) peptide;

(c) complex formed by HLA-DQ2.5 and a salmonella peptide;

(d) complex formed by HLA-DQ2.5 and a thyroperoxidase (TPO) peptide;

(e) complex formed by HLA-DQ2.5 and Mycobacterium bovis peptide;

(f) a HLA-DQ2.5 positive PBMC B cell; and

(g) a Ba/F3 cell that expresses HLA-DQ2.5.

In some embodiments, the antigen-binding molecules/domains of the invention have stronger binding activity to (at least) one, two, three, four, five, six, seven, eight, nine, ten, eleven, twelve, 13, 14, 15, 16, 17, 18, 19, or all of the complexes (1) to (20) above, compared to (at least) one, two, three, four, five, six, or all of (a) to (g) above.

The antibody has neutralizing activity against the binding between a complex formed by HLA-DQ2.5 and a gluten peptide and TCR. In other words, the antibody blocks the binding between the HLA-DQ2.5/gluten peptide complex and TCR. The binding occurs in the presence of a gluten peptide, i.e., when HLA-DQ2.5 is bound by a gluten peptide, or forms a complex with a gluten peptide. The gluten peptide is preferably any of the gliadin peptides described herein. The antibody blocks the interaction between an HLA-DQ2.5/gluten peptide complex and an HLA-DQ2.5/gluten peptide-restricted CD4+ T cell.

More preferably, the antibody blocks at least one, two, three, four, five, six, seven or all of: the interaction between an HLA-DQ2.5/33mer gliadin peptide complex and an HLA-DQ2.5/33mer gliadin peptide-restricted CD4+ T cell, the interaction between an HLA-DQ2.5/26mer gliadin peptide complex and an HLA-DQ2.5/26mer gliadin peptide-restricted CD4+ T cell, the interaction between an HLA-DQ2.5/14mer 1 peptide complex and an HLA-DQ2.5/14mer 1 peptide-restricted CD4+ T cell, the interaction between an HLA-DQ2.5/alpha 1 gliadin peptide complex and an HLADQ2.5/alpha 1 gliadin peptide-restricted CD4+ T cell, the interaction between an HLA-DQ2.5/alpha 1b gliadin peptide complex and an HLA-DQ2.5/alpha 1b gliadin peptide-restricted CD4+ T cell, the interaction between an HLA-DQ2.5/alpha 2 gliadin peptide complex and an HLA-DQ2.5/alpha 2 gliadin peptide-restricted CD4+ T cell, the interaction between an HLA-DQ2.5/alpha 3 gliadin peptide complex and an HLADQ2.5/alpha 3 gliadin peptide-restricted CD4+ T cell, the interaction between an HLA-DQ2.5/gamma 1 gliadin peptide complex and an HLA-DQ2.5/gamma 1 gliadin peptide-restricted CD4+ T cell, the interaction between an HLA-DQ2.5/gamma 2 gliadin peptide complex and an HLA-DQ2.5/gamma 2 gliadin peptide-restricted CD4+ T cell, the interaction between an HLA-DQ2.5/gamma 4b gliadin peptide complex and an HLA-DQ2.5/gamma 4b gliadin peptide-restricted CD4+ T cell, the interaction between an HLA-DQ2.5/omega 1 gliadin peptide complex and an HLA-DQ2.5/omega 1 gliadin peptide-restricted CD4+ T cell, the interaction between an HLADQ2.5/omega 2 gliadin peptide complex and an HLA-DQ2.5/omega 2 gliadin peptide-restricted CD4+ T cell, the interaction between an HLA-DQ2.5/BC hordein peptide complex and an HLA-DQ2.5/BC hordein peptide-restricted CD4+ T cell, the interaction between an HLA-DQ2.5/avenin 1 peptide complex and an HLADQ2.5/avenin 1 peptide-restricted CD4+ T cell, the interaction between an HLADQ2.5/avenin 2 peptide complex and an HLA-DQ2.5/avenin 2 peptide-restricted CD4+ T cell, the interaction between an HLA-DQ2.5/avenin 3 peptide complex and an HLA-DQ2.5/avenin 3 peptide-restricted CD4+ T cell, the interaction between an HLADQ2.5/hordein 1 peptide complex and an HLA-DQ2.5/hordein 1 peptide-restricted CD4+ T cell, the interaction between an HLA-DQ2.5/hordein 2 peptide complex and an HLA-DQ2.5/hordein 2 peptide-restricted CD4+ T cell, the interaction between an HLA-DQ2.5/secalin 1 peptide complex and an HLA-DQ2.5/secalin 1 peptide-restricted CD4+ T cell and the interaction between an HLA-DQ2.5/secalin 2 peptide complex and an HLA-DQ2.5/secalin 2 peptide-restricted CD4+ T cell.

The blocking of the interaction can be achieved by blocking of the above-mentioned binding between HLA-DQ2.5 (or the HLA-DQ2.5/gluten peptide complex) and TCR.

*The feature of the “neutralizing activity” can be defined, for example, as described herein. The antibody having the “neutralizing activity” can neutralize the binding between HLA-DQ2.5 (or the HLA-DQ2.5/gluten peptide complex) and TCR for 95% or more, preferably 97% or more, more preferably 99% or more by antibody concentration of 1 microgram (micro g)/mL under the measurement conditions described herein.

The antibody of the invention may have substantially no binding activity to (does not substantially bind to) either or both of a HLA-DQ2.5 positive PBMC B cell and a Ba/F3 cell that expresses HLA-DQ2.5. In other words, the antibody may have no specific/significant binding activity to (does not specifically/significantly bind to) either or both of a HLA-DQ2.5 positive PBMC B cell and a Ba/F3 cell that expresses HLA-DQ2.5. The meaning of the phrase “substantially no binding activity” and similar wordings is defined elsewhere herein.

Preferably, anti-HLA-DQ2.5 antibodies (antigen-binding molecules) of the present invention have specific binding activity to HLA-DQ2.5 in the form of a complex with a gluten peptide but have substantially no binding activity to HLA-DQ2.5 in the form of a complex with an irrelevant peptide, or HLA-DQ2.5 which is not in the form of a complex with a peptide.

In some embodiments, an antigen-binding molecule of the invention has binding activity to at least one, two, three, four, five, six, seven, eight, nine, or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLADQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, where the antigen-binding molecule has substantially no binding activity to either or both of a HLA-DQ2.5 positive PBMC B cell and a Ba/F3 cell that expresses HLA-DQ2.5.

In some embodiments, an antigen-binding molecule of the invention has binding activity to at least one, two, three, four, five, six, seven, eight, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, where the antigen-binding molecule has substantially no binding activity to either or both of a HLA-DQ2.5 positive PBMC B cell and a Ba/F3 cell that expresses HLA-DQ2.5.

In some embodiments, an antigen-binding molecule of the invention has binding activity to at least one, two, three, or four or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; and complex formed by HLADQ2.5 and a 14 mer 1 peptide, where the antigen-binding molecule has substantially no binding activity to either or both of a HLA-DQ2.5 positive PBMC B cell and a Ba/F3 cell that expresses HLA-DQ2.5.

In some embodiments, an antigen-binding molecule of the invention has binding activity to at least one, two, three, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; and complex formed by HLA-DQ2.5 and a 14 mer 1 peptide, where the antigen-binding molecule has substantially no binding activity to either or both of a HLA-DQ2.5 positive PBMC B cell and a Ba/F3 cell that expresses HLA-DQ2.5.

In some embodiments, an antigen-binding molecule of the invention has binding activity to at least three, four, five, six, seven, eight, nine, or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, where the antigen-binding molecule has substantially no binding activity to either or both of a HLA-DQ2.5 positive PBMC B cell and a Ba/F3 cell that expresses HLA-DQ2.5.

In some embodiments, an antigen-binding molecule of the invention has binding activity to at least three, four, five, six, seven, eight, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; complex formed by HLA-DQ2.5 and a 14 mer 1 peptide; complex formed by HLADQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, where the antigen-binding molecule has substantially no binding activity to either or both of a HLA-DQ2.5 positive PBMC B cell and a Ba/F3 cell that expresses HLA-DQ2.5.

In some embodiments, the antigen-binding molecule of the invention has substantially no binding activity to a HLA-DQ2.5 positive PBMC B cell. In some embodiments, the antigen-binding molecule of the invention has substantially no binding activity to a Ba/F3 cell that expresses HLA-DQ2.5. In some embodiments, the antigen-binding molecule of the invention has substantially no binding activity to a HLADQ2.5 positive PBMC B cell and a Ba/F3 cell that expresses HLA-DQ2.5. Herein, the absence of binding to the HLA-DQ2.5 positive PBMC B cell and/or Ba/F3 cell that expresses HLA-DQ2.5 means that the antigen-binding molecule have substantially no binding activity to HLA-DQ2.5 which is not in the form of a complex with a gluten peptide, or which is in the form of a complex with an irrelevant peptide.

In some embodiments, an antigen-binding molecule of the invention has binding activity to all of: complex formed by HLA-DQ2.5 and a BC hordein peptide; complex formed by HLA-DQ2.5 and a gamma 1 gliadin peptide; and complex formed by HLADQ2.5 and a gamma 2 gliadin peptide, where the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLADQ2.5 positive PBMC B cell; and a Ba/F3 cell that expresses HLA-DQ2.5.

In some embodiments, an antigen-binding molecule of the invention has binding activity to all of: complex formed by HLA-DQ2.5 and a BC hordein peptide; and complex formed by HLA-DQ2.5 and a gamma 1 gliadin peptide, where the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLADQ2.5.

In some embodiments, an antigen-binding molecule of the invention has binding activity to all of: complex formed by HLA-DQ2.5 and a BC hordein peptide; complex formed by HLA-DQ2.5 and a gamma 1 gliadin peptide; complex formed by HLADQ2.5 and a gamma 2 gliadin peptide; and complex formed by HLA-DQ2.5 and a 26 mer gliadin, where the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLA-DQ2.5.

In some embodiments, the antigen-binding molecule of the invention has binding activity to a complex formed by HLA-DQ2.5 and an immune dominant peptide related to celiac disease.

In some embodiments, the antigen-binding molecule of the invention has binding activity to all of: a complex formed by HLA-DQ2.5 and an immune dominant peptide related to celiac disease; complex formed by HLA-DQ2.5 and a 26 mer gliadin peptide; and complex formed by HLA-DQ2.5 and a 14 mer 1 peptide.

In some embodiments, the antigen-binding molecule of the invention has binding activity to all of: complex formed by HLA-DQ2.5 and a 33mer gliadin peptide; complex formed by HLA-DQ2.5 and an alpha 1 gliadin peptide; complex formed by HLA-DQ2.5 and an alpha 2 gliadin peptide; complex formed by HLA-DQ2.5 and a gamma 1 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and an alpha 3 gliadin peptide; complex formed by HLA-DQ2.5 and an alpha 1b gliadin peptide; complex formed by HLA-DQ2.5 and a gamma 4b gliadin peptide; complex formed by HLA-DQ2.5 and an avenin 1 peptide; complex formed by HLADQ2.5 and an avenin 2 peptide; complex formed by HLA-DQ2.5 and a hordein 1 peptide; complex formed by HLA-DQ2.5 and a hordein 2 peptide; complex formed by HLA-DQ2.5 and a secalin 1 peptide; complex formed by HLA-DQ2.5 and a secalin 2 peptide; complex formed by HLA-DQ2.5 and a 14mer 1 peptide; and complex formed by HLA-DQ2.5 and a 26mer gliadin peptide.

In some embodiments, an antigen-binding molecule of the invention has binding activity to all of: complex formed by HLA-DQ2.5 and a BC hordein peptide; complex formed by HLA-DQ2.5 and a gamma 1 gliadin peptide; and complex formed by HLADQ2.5 and a 26 mer gliadin, where the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLA-DQ2.5.

In some embodiments, the antigen-binding molecule of the invention blocks the interaction between HLA-DQ2.5/gluten peptide complex and HLA-DQ2.5/gluten peptide-restricted CD4+ T cell. In this context, the gluten peptide is the peptide in the complex bound by any of the antigen-binding molecules described above.

In some embodiments, the gluten peptide(s) is/are the following:

[1] one, two, three, four, five, six, seven, eight, or all of alpha 1 gliadin peptide, alpha 2 gliadin peptide, omega 1 gliadin peptide, omega 2 gliadin peptide, gamma 1 gliadin peptide, gamma 2 gliadin peptide, BC hordein peptide, alpha 1b gliadin peptide, and gamma 4a gliadin peptide;

[2] alpha 1 gliadin peptide, alpha 2 glaidin peptide, omega 1 gliadin peptide, and alpha 1b gliadin peptide;

[3] alpha 2 glaidin peptide, omega 2 gliadin peptide, gamma 1 gliadin peptide, gamma 2 gliadin peptide, BC hordein peptide, alpha 1b glaidin peptide, and gamma 4a gliadin peptide;

[4] alpha 2 glaidin peptide, omega 2 gliadin peptide, gamma 1 gliadin peptide, and BC hordein peptide;

[5] alpha 1 gliadin peptide, alpha 2 glaidin peptide, omega 1 gliadin peptide, omega 2 gliadin peptide, gamma 1 gliadin peptide, BC hordein peptide, alpha 1b glaidin peptide, gamma 4a gliadin peptide, and gamma 2 gliadin peptide;

[5a] alpha 1 gliadin peptide, alpha 2 glaidin peptide, omega 1 gliadin peptide, omega 2 gliadin peptide, gamma 1 gliadin peptide, BC hordein peptide, alpha 1b glaidin peptide, and gamma 4a gliadin peptide;

[6] alpha 1 gliadin peptide, alpha 2 glaidin peptide, omega 1 gliadin peptide, omega 2 gliadin peptide, gamma 1 gliadin peptide, BC hordein peptide, and alpha 1b glaidin peptide.

In some embodiments, the antigen-binding molecule of the invention has substantially no binding activity to HLA-DQ8. In some embodiments, the antigen-binding molecule of the invention has substantially no binding activity to HLA-DQ2.2, HLADQ7.5, HLA-DQ5.1, HLA-DQ6.3, or HLADQ7.3. In some embodiments, the antigen-binding molecule of the invention has substantially no binding activity to HLA-DR or HLA-DP.

In some embodiments, the antigen-binding molecule of the invention has enhanced binding activity to a complex formed by HLA-DQ2.5 and a gluten peptide. In this context, the gluten peptide may be any of the gluten peptides described above. The degree of enhancement may be determined as compared to the binding activity to a complex formed by HLA-DQ2.5 and an irrelevant peptide, or to a cell without the complex of interest, e.g., a HLA-DQ2.5 positive PBMC B cell and/or a Ba/F3 cell that expresses HLA-DQ2.5.

In some embodiments, the antigen-binding molecule of the invention has stronger binding activity to at least one, two, three, four, five, six, seven, eight, nine, or all of: complex formed by HLA-DQ2.5 and a BC hordein peptide; complex formed by HLADQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLADQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

In some embodiments, the antigen-binding molecule of the invention has stronger binding activity to at least one, two, three, four, five, six, seven, eight, or all of: complex formed by HLA-DQ2.5 and a BC hordein peptide; complex formed by HLADQ2.5 and a gamma 1 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

In some embodiments, the antigen-binding molecule of the invention has binding activity to at least one, two, three, four, five, six, seven, eight, nine, or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLADQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, where the antigen-binding molecule has substantially no binding activity to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and an HLADQ2.5 positive PBMC B cell, where the antigen-binding molecule blocks the interaction between HLA-DQ2.5/gluten peptide complex and HLA-DQ2.5/gluten peptide-restricted CD4+ T cell. In this context, the gluten peptide is the peptide in the complex bound by any of the antigen-binding molecules described above.

In some embodiments, the antigen-binding molecule of the invention has binding activity to at least one, two, three, four, five, six, seven, eight, or all of: complex formed by HLA-DQ2.5 and a BC hordein peptide; complex formed by HLA-DQ2.5 and a gamma 1 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide, where the antigen-binding molecule has substantially no binding activity to at least one, two, three, four, five or all of: complex formed by HLA-DQ2.5 and a CLIP peptide; complex formed by HLADQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and an HLADQ2.5 positive PBMC B cell, where the antigen-binding molecule blocks the interaction between HLA-DQ2.5/gluten peptide complex and HLA-DQ2.5/gluten peptide-restricted CD4+ T cell. In this context, the gluten peptide is the peptide in the complex bound by any of the antigen-binding molecules described above.

In one aspect, the invention provides an anti-HLA-DQ2.5 antibody comprising at least one, two, three, four, five, or six HVRs (CDRs) selected from (a) HVR-H1 (HCDR1) comprising the amino acid sequence of any one of SEQ ID NOs: 2, 6, 10, and 14; (b) HVR-H2 (HCDR2) comprising the amino acid sequence of any one of SEQ ID NOs: 3, 7, 11, and 15; (c) HVR-H3 (HCDR3) comprising the amino acid sequence of any one of SEQ ID NOs: 4, 8, 12, and 16; (d) HVR-L1 (LCDR1) comprising the amino acid sequence of any one of SEQ ID NOs: 18, 22, 26, and 30; (e) HVR-L2 (LCDR2) comprising the amino acid sequence of any one of SEQ ID NOs: 19, 23, 27, and 31; and (f) HVR-L3 (LCDR3) comprising the amino acid sequence of any one of SEQ ID NOs: 20, 24, 28, and 32.

In one aspect, the invention provides an antibody comprising at least one or two, or all three of the VH HVR (HCDR) sequences selected from (a) HVR-H1 (HCDR1) comprising the amino acid sequence of any one of SEQ ID NOs: 2, 6, 10, and 14; (b) HVR-H2 (HCDR2) comprising the amino acid sequence of any one of SEQ ID NOs: 3, 7, 11, and 15; and (c) HVR-H3 (HCDR3) comprising the amino acid sequence of any one of SEQ ID NOs: 4, 8, 12, and 16.

In another aspect, the invention provides an antibody comprising at least one or two, or all three of the VL HVR (LCDR) sequences selected from (a) HVR-L1 (LCDR1) comprising the amino acid sequence of any one of SEQ ID NOs: 18, 22, 26, and 30; (b) HVR-L2 (LCDR2) comprising the amino acid sequence of any one of SEQ ID NOs: 19, 23, 27, and 31; and (c) HVR-L3 (LCDR3) comprising the amino acid sequence of any one of SEQ ID NOs: 20, 24, 28, and 32.

In another aspect, an antibody of the invention comprises (a) a VH domain comprising at least one or two, or all three of the VH HVR (HCDR) sequences selected from (i) HVR-H1 (HCDR1) comprising the amino acid sequence of any one of SEQ ID NOs: 2, 6, 10, and 14, (ii) HVR-H2 (HCDR2) comprising the amino acid sequence of any one of SEQ ID NOs: 3, 7, 11, and 15, and (iii) HVR-H3 (HCDR3) comprising an amino acid sequence of any one of SEQ ID NOs: 4, 8, 12, and 16; and (b) a VL domain comprising at least one or two, or all three of the VL HVR (LCDR) sequences selected from (i) HVR-L1 (LCDR1) comprising the amino acid sequence of any one of SEQ ID NOs: 18, 22, 26, and 30, (ii) HVR-L2 (LCDR2) comprising the amino acid sequence of any one of SEQ ID NOs: 19, 23, 27, and 31, and (c) HVR-L3 (LCDR3) comprising the amino acid sequence of any one of SEQ ID NOs: 20, 24, 28, and 32.

In another aspect, the invention provides an antibody comprising (a) HVR-H1 (HCDR1) comprising the amino acid sequence of any one of SEQ ID NOs: 2, 6, 10, and 14; (b) HVR-H2 (HCDR2) comprising the amino acid sequence of any one of SEQ ID NOs: 3, 7, 11, and 15; (c) HVR-H3 (HCDR3) comprising the amino acid sequence of any one of SEQ ID NOs: 4, 8, 12, and 16; (d) HVR-L1 (LCDR1) comprising the amino acid sequence of any one of SEQ ID NOs: 18, 22, 26, and 30; (e) HVR-L2 (LCDR2) comprising the amino acid sequence of any one of SEQ ID NOs: 19, 23, 27, and 31; and (f) HVR-L3 (LCDR3) comprising an amino acid sequence selected from any one of SEQ ID NOs: 20, 24, 28, and 32.

In another aspect, the sequence ID numbers (SEQ ID NOs) of the VH, VL, HCDR1, HCDR2, HCDR3, LCDR1, LCDR2, and LCDR3 sequences for the antibodies of the present invention are as follows:

TABLE 1

| Antibody | SEQ ID NO: | | | | | | | |
|---|---|---|---|---|---|---|---|---|
| Name | VH | HCDR1 | HCDR2 | HCDR3 | VL | LCDR1 | LCDR2 | LCDR3 |
| DQN0385ee | 1 | 2 | 3 | 4 | 17 | 18 | 19 | 20 |
| DQN0429cc | 5 | 6 | 7 | 8 | 21 | 22 | 23 | 24 |
| DQN0344xx | 9 | 10 | 11 | 12 | 25 | 26 | 27 | 28 |
| DQN0139bb | 85 | 86 | 87 | 88 | 89 | 90 | 91 | 92 |
| IC17 | 13 | 14 | 15 | 16 | 29 | 30 | 31 | 32 |

In certain embodiments, the antigen-binding molecule of the invention is any one of (1) to (5) below:

(1) an antigen-binding molecule comprising the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;

(2) an antigen-binding molecule comprising the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;

(3) an antigen-binding molecule comprising the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28;

(4) an antigen-binding molecule that binds to the same epitope bound by the antigen-binding molecule of any one of (1) to (3);

(5) an antigen-binding molecule that competes with the antigen-binding molecule of any one of (1) to (3) for binding to HLA-DQ2.5 or a complex formed by HLA-DQ2.5 and a gluten peptide.

In some embodiments, the antigen-binding molecule of the invention is a bispecific antigen-binding molecule.

In some embodiments, the bispecific antigen-binding molecule of the invention is a bispecific antibody.

In certain embodiments, any one or more amino acids of an anti-HLA-DQ2.5 antibody as provided above are substituted in any of the HVR positions.

In certain embodiments, the substitutions are conservative substitutions, as provided herein.

In any of the above embodiments, an anti-HLA-DQ2.5 antibody is humanized. In one embodiment, an anti-HLA-DQ2.5 antibody comprises HVRs as in any of the above embodiments, and further comprises an acceptor human framework, e.g., a human immunoglobulin framework or a human consensus framework. In another embodiment, an anti-HLA-DQ2.5 antibody comprises HVRs as in any of the above embodiments, and further comprises the FR1, FR2, FR3, or FR4 sequence shown herein.

In another aspect, an anti-HLA-DQ2.5 antibody comprises a heavy-chain variable domain (VH) sequence having at least 90%, 91%, 92%, 93%, 94%, 95%, 96%, 97%, 98%, 99%, or 100% sequence identity to the amino acid sequence of any one of SEQ ID NOs: 1, 5, 9, and 13. In certain embodiments, a VH sequence having at least 90%, 91%, 92%, 93%, 94%, 95%, 96%, 97%, 98%, or 99% identity comprises substitutions (e.g., conservative substitutions), insertions, or deletions relative to the reference sequence, but an anti-HLA-DQ2.5 antibody comprising that sequence retains the ability to bind to HLA-DQ2.5. In certain embodiments, a total of 1 to 10 amino acids have been substituted, inserted and/or deleted in any one of SEQ ID NOs: 1, 5, 9, and 13. In certain embodiments, substitutions, insertions, or deletions occur in regions outside the HVRs (i.e., in the FRs). Optionally, the anti-HLA-DQ2.5 antibody comprises the VH sequence of any one of SEQ ID NOs: 1, 5, 9, and 13 or a sequence comprising a post-translational modification thereof. In a particular embodiment, the VH comprises one, two or three HVRs selected from: (a) HVR-H1 comprising the amino acid sequence of any one of SEQ ID NOs: 2, 6, 10, and 14, (b) HVR-H2 comprising the amino acid sequence of any one of SEQ ID NOs: 3, 7, 11, and 15, and (c) HVR-H3 comprising the amino acid sequence of any one of SEQ ID NOs: 4, 8, 12, and 16. Post-translational modifications include but are not limited to a modification of glutamine or glutamate at the N terminus of the heavy chain or light chain to pyroglutamic acid by pyroglutamylation.

In another aspect, an anti-HLA-DQ2.5 antibody is provided, wherein the antibody comprises a light-chain variable domain (VL) having at least 90%, 91%, 92%, 93%, 94%, 95%, 96%, 97%, 98%, 99%, or 100% sequence identity to the amino acid sequence of any one of SEQ ID NOs: 17, 21, 25, and 29. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-HLA-DQ2.5 antibody comprising that sequence retains the ability to bind to HLA-DQ2.5. In certain embodiments, a total of 1 to 10 amino acids have been substituted, inserted and/or deleted in any one of SEQ ID NOs: 17, 21, 25, and 29. In certain embodiments, the substitutions, insertions, or deletions occur in regions outside the HVRs (i.e., in the FRs). Optionally, the anti-HLA-DQ2.5 antibody comprises the VL sequence of any one of SEQ ID NOs: 17, 21, 25, and 29 or a sequence comprising a post-translational modification thereof. In a particular embodiment, the VL comprises one, two or three HVRs selected from (a) HVR-L1 comprising the amino acid sequence of any one of SEQ ID NOs: 18, 22, 26, and 30; (b) HVR-L2 comprising the amino acid sequence of any one of SEQ ID NOs: 19, 23, 27, and 31; and (c) HVR-L3 comprising the amino acid sequence of any one of SEQ ID NOs: 20, 24, 28, and 32. Post-translational modifications include but are not limited to a modification of glutamine or glutamate at the N terminus of the heavy chain or light chain to pyroglutamic acid by pyroglutamylation.

In another aspect, an anti-HLA-DQ2.5 antibody is provided, wherein the antibody comprises a VH as in any of the embodiments provided above, and a VL as in any of the embodiments provided above. In one embodiment, the antibody comprises the VH sequence of any one of SEQ ID NOs: 1, 5, 9, and 13 or a sequence comprising a post-translational modification thereof, and the VL sequence of any one of SEQ ID NOs: 17, 21, 25, and 29 or a sequence comprising a post-translational modification thereof. Post-translational modifications include but are not limited to a modification of glutamine or glutamate at the N terminus of the heavy chain or light chain to pyroglutamic acid by pyroglutamylation.

In a further aspect, the invention provides an antibody that binds to the same epitope as an anti-HLA-DQ2.5 antibody provided herein. For example, in certain embodiments, an antibody is provided that binds to the same epitope as any of the antibodies described herein. In certain embodiments, an antibody is provided that binds to an epitope within a fragment of HLA-DQ2.5 consisting of about 8 to 17 amino acids, or within a complex formed by HLA-DQ2.5 and a gluten peptide. In this context, the gluten peptide may be any of the gluten peptides described herein.

In a further aspect, the invention provides an antibody that competes with another antibody for binding to HLA-DQ2.5 or a complex formed by HLA-DQ2.5 and a gluten peptide. For example, in certain embodiments, an antibody is provided that competes with any of the antibodies described herein for binding to HLA-DQ2.5 or a complex formed by HLA-DQ2.5 and a gluten peptide. In this context, the gluten peptide may be any of the gluten peptides described herein.

In a further aspect of the invention, an anti-HLA-DQ2.5 antibody according to any of the above embodiments is a monoclonal antibody, including a chimeric, humanized or human antibody. In one embodiment, an anti-HLA-DQ2.5 antibody is an antibody fragment, e.g., a Fv, Fab, Fab′, scFv, diabody, or F(ab′)₂ fragment. In another embodiment, the antibody is a full-length antibody, e.g., an intact IgG1 antibody or other antibody class or isotype as defined herein.

In a further aspect, an anti-HLA-DQ2.5 antibody according to any of the above embodiments may incorporate any of the features described below, whether singly or in combination:

1. Antibody Affinity

In certain embodiments, an antibody provided herein has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE (registered trademark)-3000 (BIAcore, Inc., Piscataway, N.J.) is performed at 25 degrees C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (approximately 0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB (registered trademark) technology; U.S. Pat. No. 7,041,870 describing K-M MOUSE (registered trademark) technology, and U.S. Patent Application Publication No. US 2007/0061900, describing VELOCIMOUSE (registered trademark) technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

a) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

b) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Fc Region

The term “Fc region” or “Fc domain” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma RIb and Fc gamma RIc; Fc gamma RII (CD32) including isoforms Fc gamma RIIa (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIIIa (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RII (CD32), Fc gamma RIII (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fc gamma RI (CD64), Fc gamma RIIA (CD32), Fc gamma RIIB (CD32), Fc gamma RIIIA (CD16), and/or Fc gamma RIIIB (CD16). The polynucleotide sequence and amino acid sequence of Fc gamma RI are shown in SEQ ID NOs: 80 (NM_000566.3) and 74 (NP_000557.1), respectively; the polynucleotide sequence and amino acid sequence of Fc gamma RIIA are shown in SEQ ID NOs: 81 (BC020823.1) and 75 (AAH20823.1), respectively; the polynucleotide sequence and amino acid sequence of Fc gamma RIIB are shown in SEQ ID NOs: 82 (BC146678.1) and 76 (AAI46679.1), respectively; the polynucleotide sequence and amino acid sequence of Fc gamma RIIIA are shown in SEQ ID NOs: 83 (BC033678.1) and 77 (AAH33678.1), respectively; and the polynucleotide sequence and amino acid sequence of Fc gamma RIIIB are shown in SEQ ID NOs: 84 (BC128562.1) and 78 (AAI28563.1), respectively (RefSeq accession number is shown in each parentheses). Whether an Fc gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fc gamma receptor, Fc alpha receptor, Fc beta receptor, FcRn, C1q, and C3, mannan-binding lectin, mannose receptor, Staphylococcus Protein A, Staphylococcus Protein G, and viral Fc gamma receptors. The Fc ligands also include Fc receptor homologs (FcRH) (Davis et al., (2002) Immunological Reviews 190, 123-136), which are a family of Fc receptors homologous to Fc gamma receptor. The Fc ligands also include unidentified molecules that bind to Fc.

Fc Gamma Receptor-Binding Activity

The impaired binding activity of Fc domain to any of the Fc gamma receptors Fc gamma RI, Fc gamma RIIA, Fc gamma RIIB, Fc gamma RIIIA, and/or Fc gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fe domain, inducing a signal of 520 to 620 nm as a result. The antigen-binding molecule or antibody having a non-tagged mutant Fc domain competes with the antigen-binding molecule or antibody comprising a wild-type Fc domain for the interaction with Fc gamma receptor. The relative binding affinity can be determined by quantifying the reduction of fluorescence as a result of competition. Methods for biotinylating the antigen-binding molecules or antibodies such as antibodies using Sulfo-NHS-biotin or the like are known. Appropriate methods for adding the GST tag to an Fc gamma receptor include methods that involve fusing polypeptides encoding Fc gamma receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE methods. Examples of such inhibition assay are described in Proc. Natl. Acad. Sci. USA (2006) 103(11), 4005-4010.

Fc Region with a Reduced Fc Gamma Receptor-Binding Activity

Herein, “a reduced Fc gamma receptor-binding activity” means, for example, that based on the above-described analysis method the competitive activity of a test antigen-binding molecule or antibody is 50% or less, preferably 45% or less, 40% or less, 35% or less, 30% or less, 20% or less, or 15% or less, and particularly preferably 10% or less, 9% or less, 8% or less, 7% or less, 6% or less, 5% or less, 4% or less, 3% or less, 2% or less, or 1% or less than the competitive activity of a control antigen-binding molecule or antibody.

In the present invention, SG181 may be used as a Fe gamma receptor silenced Fc which attenuates Fc binding against Fc gamma receptors. In some embodiments, SG181.S3n (SEQ ID NO: 33) and SG181.S3p (SEQ ID NO: 34) may be used as heavy chain constant regions sequences. These heavy chain constant regions sequences may be included in the antigen-binding molecules or antibodies of the present invention for reduced Fc gamma receptor binding.

Antigen-binding molecules or antibodies comprising the Fc domain of a monoclonal IgG1, IgG2, IgG3, or IgG4 antibody can be appropriately used as control antigen-binding molecules or antibodies. The Fc domain structures are shown in SEQ ID NOs: 62 (A is added to the N terminus of RefSeq accession number AAC82527.1), 63 (A is added to the N terminus of RefSeq accession number AAB59393.1), 64 (A is added to the N terminus of RefSeq accession number CAA27268.1), and 65 (A is added to the N terminus of RefSeq accession number AAB59394.1). Furthermore, when an antigen-binding molecule or antibody comprising an Fc domain mutant of an antibody of a particular isotype is used as a test substance, the effect of the mutation of the mutant on the Fc gamma receptor-binding activity is assessed using as a control an antigen-binding molecule or antibody comprising an Fc domain of the same isotype. As described above, antigen-binding molecules or antibodies comprising an Fc domain mutant whose Fc gamma receptor-binding activity has been judged to be reduced are appropriately prepared.

Such known mutants include, for example, mutants having a deletion of amino acids 231A-238S (EU numbering) (WO 2009/011941), as well as mutants C226S, C229S, P238S, (C220S) (J. Rheumatol (2007) 34, 11); C226S and C229S (Hum. Antibod. Hybridomas (1990) 1(1), 47-54); C226S, C229S, E233P, L234V, and L235A (Blood (2007) 109, 1185-1192).

Specifically, the preferred antigen-binding molecules or antibodies include those comprising an Fc domain with a mutation (such as substitution) of at least one amino acid selected from the following amino acid positions: 220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, or 332 (EU numbering), in the amino acids forming the Fc domain of an antibody of a particular isotype. The isotype of antibody from which the Fc domain originates is not particularly limited, and it is possible to use an appropriate Fc domain derived from a monoclonal IgG1, IgG2, IgG3, or IgG4 antibody. It is preferable to use Fc domains derived from IgG1 antibodies.

The preferred antigen-binding molecules or antibodies include, for example, those comprising an Fc domain which has any one of the substitutions shown below, whose positions are specified according to EU numbering (each number represents the position of an amino acid residue in the EU numbering; and the one-letter amino acid symbol before the number represents the amino acid residue before substitution, while the one-letter amino acid symbol after the number represents the amino acid residue after the substitution) in the amino acids forming the Fc domain of IgG1 antibody:

### (a) L234F, L235E, P331S;

### (b) C226S, C229S, P238S;

### (c) C226S, C229S; or

### (d) C226S, C229S, E233P, L234V, L235A;

as well as those having an Fc domain which has a deletion of the amino acid sequence at positions 231 to 238.

Furthermore, the preferred antigen-binding molecules or antibodies also include those comprising an Fc domain that has any one of the substitutions shown below, whose positions are specified according to EU numbering in the amino acids forming the Fc domain of an IgG2 antibody:

### (e) H268Q, V309L, A330S, and P331S;

### (f) V234A;

### (g) G237A;

### (h) V234A and G237A;

### (i) A235E and G237A; or

(j) V234A, A235E, and G237A. Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG3 antibody:

### (k) F241A;

### (l) D265A; or

(m) V264A. Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG4 antibody:

### (n) L235A, G237A, and E318A;

### (o) L235E; or

(p) F234A and L235A. Each number represents the position of an amino acid residue in EU numbering; and the one-letter amino acid symbol before the number represents the amino acid residue before substitution, while the one-letter amino acid symbol after the number represents the amino acid residue after the substitution.

The other preferred antigen-binding molecules or antibodies include, for example, those comprising an Fc domain in which any amino acid at position 233, 234, 235, 236, 237, 327, 330, or 331 (EU numbering) in the amino acids forming the Fc domain of an IgG1 antibody is substituted with an amino acid of the corresponding position in EU numbering in the corresponding IgG2 or IgG4.

The preferred antigen-binding molecules or antibodies also include, for example, those comprising an Fc domain in which any one or more of the amino acids at positions 234, 235, and 297 (EU numbering) in the amino acids forming the Fc domain of an IgG1 antibody is substituted with other amino acids. The type of amino acid after substitution is not particularly limited; however, the antigen-binding molecules or antibodies comprising an Fc domain in which any one or more of the amino acids at positions 234, 235, and 297 are substituted with alanine are particularly preferred.

The preferred antigen-binding molecules or antibodies also include, for example, those comprising an Fc domain in which an amino acid at position 265 (EU numbering) in the amino acids forming the Fc domain of an IgG1 antibody is substituted with another amino acid. The type of amino acid after substitution is not particularly limited; however, antigen-binding molecules or antibodies comprising an Fc domain in which an amino acid at position 265 is substituted with alanine are particularly preferred.

c) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

d) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Antibodies may be produced using recombinant methods and compositions, e.g., as described in U.S. Pat. No. 4,816,567. In one embodiment, isolated nucleic acid encoding an anti-HLA-DQ2.5 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making an anti-HLA-DQ2.5 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-HLA-DQ2.5 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

C. Assays

Anti-HLA-DQ2.5 antibodies provided herein may be identified, screened for, or characterized for their physical/chemical properties and/or biological activities by various assays known in the art.

Binding Assays and Other Assays

In one aspect, an antibody of the invention is tested for its antigen binding activity, e.g., by known methods such as ELISA, Western blot, etc.

In another aspect, competition assays may be used to identify an antibody that competes with, for example, any of the above-mentioned antibodies for binding to HLA-DQ2.5 (or HLA-DQ2.5/gluten peptide complex). In certain embodiments, such a competing antibody binds to the same epitope (e.g., a linear or a conformational epitope) that is bound by the above-mentioned antibodies. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized HLA-DQ2.5 (or HLADQ2.5/gluten peptide complex) is incubated in a solution comprising a first labeled antibody that binds to HLA-DQ2.5 (or HLA-DQ2.5/gluten peptide complex) and a second unlabeled antibody that is being tested for its ability to compete with the first antibody for binding to HLA-DQ2.5 (or HLA-DQ2.5/gluten peptide complex). The second antibody may be present in a hybridoma supernatant. As a control, immobilized HLA-DQ2.5 (or HLA-DQ2.5/gluten peptide complex) is incubated in a solution comprising the first labeled antibody but not the second unlabeled antibody. After incubation under conditions permissive for binding of the first antibody to HLA-DQ2.5 (or HLA-DQ2.5/gluten peptide complex), excess unbound antibody is removed, and the amount of label associated with immobilized HLA-DQ2.5 (or HLA-DQ2.5/gluten peptide complex) is measured. If the amount of label associated with immobilized HLA-DQ2.5 (or HLA-DQ2.5/gluten peptide complex) is substantially reduced in the test sample relative to the control sample, then that indicates that the second antibody is competing with the first antibody for binding to HLA-DQ2.5 (or HLA-DQ2.5/gluten peptide complex). See Harlow and Lane (1988) Antibodies: A Laboratory Manual ch. 14 (Cold Spring Harbor Laboratory, Cold Spring Harbor, N.Y.).

Animals such as rabbits, mice, rats, and other animals suitable for immunization are immunized with an antigen (e.g., HLA-DQ2.5 or HLA-DQ2.5/gluten peptide complex). The antigen may be prepared as a recombinant protein using any methods, for example, as mentioned herein. Antibody-containing samples such as the blood and spleen are collected from the immunized animals. For B cell selection, for example, a biotinylated antigen is prepared, and antigen-binding B cells are bound by the biotinylated antigen, and the cells are subjected to cell sorting and culturing for selection. Specific binding of the cells to the antigen may be evaluated by any suitable method such as the ELISA method. This method may also be used for assessing the absence of cross-reactivity towards antigens of no interest. To isolate or determine the sequence of the selected antibody, for example, RNAs are purified from the cells, and DNAs encoding regions of the antibody are prepared by reverse transcription of the RNAs and PCR amplification. Furthermore, cloned antibody genes may be expressed in suitable cells, and the antibody may be purified from the culture supernatants for further analysis.

To test whether an anti-HLA-DQ2.5 antibody binds to an antigen of interest (e.g., a complex formed by HLA-DQ2.5 and a gluten peptide such as those described herein), any methods for assessing the binding can be used. For example, when an FACS-based cell sorting method is used, cells expressing the antigen are incubated with the tested antibody, and then a suitable secondary antibody against the tested (i.e., primary) antibody is added and incubated. The binding between the antigen and the tested antibody is detected by FACS analysis using, for example, a chromogenic/fluorescent label attached to the secondary antibody (for example, as mentioned herein). Alternatively, any of the measurement methods mentioned in “1. Antibody Affinity” in this specification can be utilized. For example, the measurement of Kd by a BIACORE surface plasmon resonance assay can be used for assessing the binding between the tested antibody and the antigen of interest mentioned herein).

In certain embodiments, the method of the present invention further comprises: testing whether the antibody has neutralizing activity against the binding between HLA-DQ2.5 (or HLA-DQ2.5/gluten peptide complex) and TCR (or the interaction between HLA-DQ2.5 (or HLA-DQ2.5/gluten peptide complex) and HLADQ2.5-restricted CD4+ T cells); and selecting the antibody that has the neutralizing activity. These steps can be performed in the presence of a gluten peptide such as those described herein, i.e., using HLA-DQ2.5 bound by the peptide. The neutralizing activity can be assessed, for example, as mentioned herein. Briefly, beads such as streptavidin-coated yellow particles are appropriately prepared, and soluble HLA-DQ bound by a peptide is added to the beads for immobilization on a plate. The plate is washed and blocked, and the antibody is added thereto and incubated. When the binding between HLA-DQ2.5 (or HLA-DQ2.5/gluten peptide complex) and TCR is assessed, for example, D2 TCR tetramer-PE may be added and incubated. Binding between the two may be evaluated based on the chromogenic/fluorescent label of TCR bound by HLA-DQ2.5 (or HLA-DQ2.5/gluten peptide complex).

Multispecific Antigen-Binding Molecules/Antibodies

In the context of the present invention, the term “multispecific antibody (antigen-binding molecules)” refers to an antibody that may bind specifically to different types of epitopes. More specifically, multispecific antibodies are antibodies having specificity to at least two different types of epitopes, and, in addition to antibodies recognizing different antigens, antibodies recognizing different epitopes on the same antigen are also included. For example, when the antigens are heterologous receptors, multispecific antibodies can recognize different domains constituting the heterologous receptors; alternatively, when the antigens are monomers, multispecific antibodies recognize multiple sites on the monomer antigens. Ordinarily, such molecules bind to two antigens or epitopes (“bispecific antibodies”; used in the present description to have the same meaning as “dual-specific antibodies”), but they may even have specificity toward more antigens or epitopes (for example, three or more types of antigens). Herein, the terms such as “bispecific” and “multispecific” means that the specificity of an antigen-binding domain/region is different from the specificity of another antigen-binding domain/region. That is, the terms mean that there are two or more specificities in an antigen-binding molecule. For example, in a “bispecific” antibody, a first antigen-binding domain may bind to a first group of complexes formed by HLA-DQ2.5 and a gluten peptide, and the second antigen-binding domain may bind to a second group of complexes formed by HLA-DQ2.5 and a gluten peptide. The members (i.e., complexes) of the two groups may overlap but may not be identical. That is, some complexes may be included in both of the groups. The terms such as “bispecific” and “multispecific” can cover this situation. The same applies to first and second groups of complexes that are not bound by the first/second antigen-binding domain.

A multispecific antibody may comprise at least two antigen-binding domains. A bispecific antibody may comprise a first antigen-binding domain and a second antigen-binding domain. In the present invention, preferably, a bispecific antibody comprises a first antigen-binding domain which binds to one or more complexes formed by HLADQ2.5 and a gluten peptide, and a second antigen-binding domain which binds to one or more complexes formed by HLA-DQ2.5 and a gluten peptide. In this context, preferably, at least one gluten peptide in the complexes bound by the first antigen-binding domain is different from at least one gluten peptide in the complexes bound by the second antigen-binding domain. In other words, the members of the gluten peptides in the complexes bound by the first antigen-binding domain and the members of the gluten peptides in the complexes bound by the second antigen-binding domain may overlap but not be completely identical. The gluten peptides in the complexes bound by the first/second antigen-binding domain may be selected from any gluten peptides described herein. Preferably, the first/second antigen-binding domain is capable of binding to one type of gluten peptide, or two or more types of gluten peptides.

In some embodiments, an antigen-binding molecule of the invention comprises at least two antigen-binding domains, wherein either of the antigen-binding domains has binding activity to one or more complexes formed between HLA-DQ2.5 and an immune dominant peptide related to celiac disease, where either of the antigen-binding domains has substantially no binding activity to at least one, two, three, four, five or all of: complex formed by HLA-DQ2.5 and CLIP peptide; complex formed by HLADQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLADQ2.5 positive PBMC B cell. In some embodiments, the antigen-binding molecule is a bispecific or multispecific antigen-binding molecule.

In some embodiments, an antigen-binding molecule of the invention comprises at least two antigen-binding domains, where either of the antigen-binding domains has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLADQ2.5 and an omega 2 gliadin peptide; complex formed by HLA-DQ2.5 and a BC hordein peptide, where either of the antigen-binding domains has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell. In some embodiments, the antigen-binding molecule is a bispecific or multispecific antigen-binding molecule.

In some embodiments, an antigen-binding molecule of the invention comprises at least two antigen-binding domains, where either of the antigen-binding domains has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLADQ2.5 and an omega 2 gliadin peptide; complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide, where either of the antigen-binding domains has no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLADQ2.5 positive PBMC B cell. In some embodiments, the antigen-binding molecule is a bispecific or multispecific antigen-binding molecule.

In some embodiments, an antigen-binding molecule of the invention comprises at least two antigen-binding domains, where each of the above antigen-binding domains has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLADQ2.5 and an omega 2 gliadin peptide; complex formed by HLA-DQ2.5 and a BC hordein peptide; and complex formed by HLA-DQ2.5 and a gamma 1 gliadin peptide, where either of the antigen-binding domains has no binding activity to at least one, two, three, four, five, or all of: complex formed by HLA-DQ2.5 and CLIP peptide; complex formed by HLA-DQ2.5 and a salmonella peptide; complex formed by HLADQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell. In some embodiments, the antigen-binding molecule is a bispecific or multispecific antigen-binding molecule.

In some embodiments, an antigen-binding molecule of the invention comprises at least two antigen-binding domains, where each of the above antigen-binding domains has binding activity to all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLADQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLADQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide, where either of the antigen-binding domains has no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell. In some embodiments, the antigen-binding molecule is a bispecific or multispecific antigen-binding molecule.

In some embodiments, an antigen-binding molecule of the invention comprises at least two antigen-binding domains, where each of the above antigen-binding domains has binding activity to all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLADQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide, where either of the antigen-binding domains has no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell. In some embodiments, the antigen-binding molecule is a bispecific or multispecific antigen-binding molecule.

In some embodiments, an antigen-binding molecule of the invention comprises a first antigen-binding domain and a second antigen-binding domain, where the first antigen-binding domain has binding activity to one or more complexes formed by HLA-DQ2.5 and a gluten peptide, where the second antigen-binding domain has binding activity to one or more complexes formed by HLA-DQ2.5 and a gluten peptide, where at least one gluten peptide in the complexes bound by the first antigen-binding domain is different from at least one gluten peptide bound by the second antigen-binding domain.

In some embodiments, an antigen-binding molecule of the invention has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide.

In some embodiments, an antigen-binding molecule of the invention has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; and complex formed by HLA-DQ2.5 and a gamma 1 gliadin peptide.

In some embodiments, an antigen-binding molecule of the invention has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide, where the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

In some embodiments, an antigen-binding molecule of the invention has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; and complex formed by HLA-DQ2.5 and a gamma 1 gliadin peptide, where the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

In some embodiments, an antigen-binding molecule of the invention comprises a first antigen-binding domain which has binding activity to a complex formed by HLADQ2.5 and a first gluten peptide, and a second antigen-binding domain which has binding activity to a complex formed by HLA-DQ2.5 and a second gluten peptide, where the antigen-binding molecule has binding activity to at least two or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLADQ2.5 and a gamma 2 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLADQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; and complex formed by HLA-DQ2.5 and a gamma 4b gliadin peptide, where the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

In some embodiments, an antigen-binding molecule of the invention comprises a first antigen-binding domain which has binding activity to a complex formed by HLADQ2.5 and a first gluten peptide, and a second antigen-binding domain which has binding activity to a complex formed by HLA-DQ2.5 and a second gluten peptide, where the antigen-binding molecule has binding activity to at least two or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLADQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLADQ2.5 and a hordein 2 peptide; and complex formed by HLA-DQ2.5 and a gamma 4b gliadin peptide, where the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

In some embodiments, an antigen-binding molecule of the invention comprises a first antigen-binding domain and a second antigen-binding domain, where the first antigen-binding domain has binding activity to at least one or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLADQ2.5 and a secalin 1 peptide; complex formed by HLA-DQ2.5 and a secalin 2 peptide; complex formed by HLA-DQ2.5 and a 33 mer gliadin peptide; where the second antigen-binding domain has binding activity to at least one or more of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26mer gliadin peptide; complex formed by HLA-DQ2.5 and a 14mer 1 peptide; complex formed by HLADQ2.5 and a 33 mer gliadin peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide, where the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell. In some embodiments, the complex bound by the first antigen-binding domain and the complex bound by the second antigen-binding domain are different from each other.

In some embodiments, an antigen-binding molecule of the invention comprises a first antigen-binding domain and a second antigen-binding domain, where the first antigen-binding domain has binding activity to at least one or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLADQ2.5 and a secalin 1 peptide; complex formed by HLA-DQ2.5 and a secalin 2 peptide; complex formed by HLA-DQ2.5 and a 33 mer gliadin peptide; where the second antigen-binding domain has binding activity to at least one or more of: complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and a 33 mer gliadin peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLADQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide, where the antigen-binding molecule has substantially no binding activity to at least one, two, three, four, five or all of: complex formed by HLA-DQ2.5 and CLIP peptide; complex formed by HLADQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLADQ2.5 positive PBMC B cell. In some embodiments, the complex bound by the first antigen-binding domain and the complex bound by the second antigen-binding domain are different from each other.

In some embodiments, the antigen-binding molecule of the invention blocks the interaction between HLA-DQ2.5/gluten peptide complex and HLADQ2.5/gluten peptide-restricted CD4+ T cell. In this context, the gluten peptide is the peptide in the complex bound by any of the antigen-binding molecules/domains described above.

In some embodiments, the antigen-binding molecule of the invention has substantially no binding activity to HLA-DQ2.2, HLA-DQ7.5, HLA-DQ5.1, HLA-DQ6.3, or HLADQ7.3. In some embodiments, the antigen-binding molecule of the invention has substantially no binding activity to HLA-DR, or HLA-DP.

In some embodiments, the antigen-binding molecule of the invention has substantially no binding activity to HLA-DQ8.

In some embodiments, the antigen-binding molecule of the invention has enhanced binding activity to a complex formed by HLA-DQ2.5 and a gluten peptide. In this context, the gluten peptide may be any of the gluten peptides described above. The degree of enhancement may be determined as compared to the binding activity to a complex formed by HLA-DQ2.5 and an irrelevant peptide, or to a cell without the complex of interest, e.g., a HLA-DQ2.5 positive PBMC B cell and/or a Ba/F3 cell that expresses HLA-DQ2.5

In some embodiments, the antigen-binding molecule of the invention has stronger binding activity to at least two, three, four, five, six, seven, eight, nine, ten, eleven, twelve, 13, 14, 15, 16, 17, 18, or all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLADQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

In some embodiments, the antigen-binding molecule has stronger binding activity to at least two, three, four, five, six, seven, eight, nine, ten, eleven, twelve, 13, 14, 15, 16, 17, or all of: complex formed by HLA-DQ2.5 and an alpha 1 gliadin peptide; complex formed by HLA-DQ2.5 and an alpha 1b gliadin peptide; complex formed by HLADQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLADQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

The bispecific antibody of the invention comprises a heavy chain and a light chain of a first half-antibody and a heavy chain and a light chain of a second half-antibody. In some embodiments, the bispecific antibody comprises VH and VL of a first half-antibody and VH and VL of a second half-antibody. In some embodiments, the bispecific antibody comprises HCDR1, HCDR2, HCDR3, LCDR1, LCDR2, and LCDR3 of a first half-antibody and HCDR1, HCDR2, HCDR3, LCDR1, LCDR2, and LCDR3 of a second half-antibody.

In some embodiments, the first half-antibody is derived from the DQN0344xx. In some embodiments, the second half-antibody is derived from DQN0385ee or DQN0429cc. The sequences of VH, VL, HCDR1, HCDR2, HCDR3, LCDR1, LCDR2, and LCDR3 of the (half-)antibodies are described herein elsewhere, e.g., in Table 1.

In some embodiments, the antigen-binding molecule of the invention is any one of (1) to (5) below:

(1) an antigen-binding molecule comprising the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;

(2) an antigen-binding molecule comprising the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;

(3) an antigen-binding molecule comprising the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28;

(4) an antigen-binding molecule that binds to the same epitope bound by the antigen-binding molecule of any one of (1) to (3);

(5) an antigen-binding molecule that competes with the antigen-binding molecule of any one of (1) to (3) for binding to HLA-DQ2.5 or a complex formed by HLA-DQ2.5 and a gluten peptide.

In some embodiments, the antigen-binding molecule of the invention is a bispecific antigen-binding molecule.

In some embodiments, the bispecific antigen-binding molecule is a bispecific antibody.

In some embodiments, the antigen-binding molecule of the present invention is any one of (a) to (d) below:

(a) an antigen-binding molecule comprising (i) and (iii) below,

(b) an antigen-binding molecule comprising (ii) and (iii) below,

(c) an antigen-binding molecule that binds to the same epitope bound by the antigen-binding molecule of (a) or (b),

(d) an antigen-binding molecule that competes with the antigen-binding molecule of (a) or (b) for binding to HLA-DQ2.5 or a complex formed by HLA-DQ2.5 and a gluten peptide,

(i) the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;

(ii) the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;

(iii) the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28.

In some embodiments, the invention provides a nucleic acid encoding the antigen-binding molecule of the invention.

In some embodiments, the invention provides a vector into which the above-mentioned nucleic acid is introduced.

In some embodiments, the invention provides a cell comprising the above-mentioned nucleic acid or the above-mentioned vector.

In some embodiments, the invention provides a method of producing an antigen-binding molecule by culturing the above-mentioned cell.

The nucleic acid, vector, cell, and method can be suitably made/performed in view of the present disclosure and technical knowledge in the art.

In some embodiments, the present invention provides an antigen-binding molecule of any one of (1) to (5) below:

(1) an antigen-binding molecule comprising the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;

(2) an antigen-binding molecule comprising the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;

(3) an antigen-binding molecule comprising the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28;

(4) an antigen-binding molecule that binds to the same epitope bound by the antigen-binding molecule of any one of (1) to (3);

(5) an antigen-binding molecule that competes with the antigen-binding molecule of any one of (1) to (3) for binding to HLA-DQ2.5 or a complex formed by HLA-DQ2.5 and a gluten peptide.

In some embodiments, the antigen-binding molecule of the present invention is any one of (a) to (d) below:

(a) an antigen-binding molecule comprising (i) and (iii) below,

(b) an antigen-binding molecule comprising (ii) and (iii) below,

(c) an antigen-binding molecule that binds to the same epitope bound by the antigen-binding molecule of (a) or (b),

(d) an antigen-binding molecule that competes with the antigen-binding molecule of (a) or (b) for binding to HLA-DQ2.5 or a complex formed by HLA-DQ2.5 and a gluten peptide,

(i) the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;

(ii) the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;

(iii) the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28.

### OTHER EMBODIMENTS

1) Immunoconjugates

The invention also provides immunoconjugates comprising an anti-HLA-DQ2.5 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu. When the radioconjugate is used for detection, it may comprise a radioactive atom for scintigraphic studies, for example Tc-99m or ¹²³I, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionuclide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

2) Pharmaceutical Formulations

Pharmaceutical formulations of an anti-HLA-DQ2.5 antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide a drug that might be combined with the anti-HLA-DQ2.5 antibody. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

3) Therapeutic Methods and Compositions

Any of the anti-HLA-DQ2.5 antibodies provided herein may be used in therapeutic methods. In one aspect, an anti-HLA-DQ2.5 antibody for use as a medicament is provided. In further aspects, an anti-HLA-DQ2.5 antibody for use in treating celiac disease is provided. In certain embodiments, an anti-HLA-DQ2.5 antibody for use in a method of treatment is provided. In certain embodiments, the invention provides an anti-HLA-DQ2.5 antibody for use in a method of treating an individual having celiac disease comprising administering to the individual an effective amount of the anti-HLA-DQ2.5 antibody. In one such embodiment, the method further comprises administering to the individual an effective amount of at least one additional therapeutic agent, e.g., as described below.

In a further aspect, the invention provides for the use of an anti-HLA-DQ2.5 antibody in the manufacture or preparation of a medicament. In one embodiment, the medicament is for treatment of celiac disease. In a further embodiment, the medicament is for use in a method of treating celiac disease comprising administering to an individual having celiac disease an effective amount of the medicament. In one such embodiment, the method further comprises administering to the individual an effective amount of at least one additional therapeutic agent, e.g., as described below.

In a further aspect, the invention provides a method for treating a celiac disease. In one embodiment, the method comprises administering to an individual having caliac disease an effective amount of an anti-HLA-DQ2.5 antibody. In one such embodiment, the method further comprises administering to the individual an effective amount of at least one additional therapeutic agent, as described below. An “individual” according to any of the above embodiments may be a human.

In a further aspect, the invention provides pharmaceutical formulations comprising any of the anti-HLA-DQ2.5 antibodies provided herein, e.g., for use in any of the above therapeutic methods for celiac disease. In one embodiment, a pharmaceutical formulation comprises any of the anti-HLA-DQ2.5 antibodies provided herein and a pharmaceutically acceptable carrier. In another embodiment, a pharmaceutical formulation comprises any of the anti-HLA-DQ2.5 antibodies provided herein and at least one additional therapeutic agent, e.g., as described below.

Antibodies of the invention can be used either alone or in combination with other agents in a therapy. For instance, an antibody of the invention may be co-administered with at least one additional therapeutic agent. In certain embodiments, an additional therapeutic agent is any agent which is suitable for co-administration and available to those skilled in the art.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-HLA-DQ2.5 antibody and administration of an additional therapeutic agent occur within about one month, or within about one, two or three weeks, or within about one, two, three, four, five, or six days, of each other.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Two or more of the antibodies of the invention (i.e., two or more therapeutic agents of the invention) may be administered in a course of treatment. They may be administered separately or simultaneously. They may be administered concomitantly. In concomitant administration, two or more antibodies may be administered simultaneously or separately. In some cases, a certain antibody/agent may be administered first; and the symptom may be monitored; and depending on the symptom, if necessary, another antibody/agent may be further administered. Alternatively, two or more antibodies of the invention may be contained in a combination drug/agent. Such a combination drug/agent may be administered as described herein. The dose/dosage of each antibody contained may be suitably determined as mentioned herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 micro g/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. The progress of this therapy is easily monitored by conventional techniques and assays.

It is understood that any of the above formulations or therapeutic methods may be carried out using an immunoconjugate of the invention in place of or in addition to an anti-HLA-DQ2.5 antibody.

4) Articles of Manufacture

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-HLA-DQ2.5 antibody.

5) Methods of Using Antigen-Binding Molecules

The antigen-binding molecules of the present disclosure can be combined with techniques of various, preexisting medical use. Non-limiting examples of techniques that can be combined with the antigen-binding molecules of the present disclosure include methods of incorporating a nucleic acid encoding an antigen-binding molecule into the living body using a viral vector or such, and directly expressing the antigen-binding molecule. Examples of such viral vectors include, but not limited to, adenovirus. Alternatively, it is possible to directly incorporate a nucleic acid encoding an antigen-binding molecule into the living body by, for example, an electroporation method or a method of directly administering a nucleic acid, without using a viral vector. Alternatively, it is possible to administer a cell genetically modified to secrete/express the antigen-binding molecule to the living body, and allow the antigen-binding molecule to be continuously secreted in the living body.

Although the invention will be described in some detail by way of illustration and example for purposes of clarity of understanding, the descriptions and examples should not be construed as limiting the scope of the invention. The disclosures of all patent and scientific literature cited herein are expressly incorporated in their entirety by reference.

### EXAMPLES

The following are examples of compositions of the invention. It is understood that various other embodiments may be practiced, given the general description provided above.

### Example 1

Expression and Purification of Recombinant Proteins

1.1. Expression and Purification of Recombinant HLA-DQ2.5/33mer Gliadin Peptide Complex, HLA-DQ8/Gliadin Peptide Complex, HLA-DQ5.1/DBY Peptide Complex,

HLA-DQ2.2/CLIP Peptide Complex, HLA-DQ7.5/CLIP Peptide Complex, HLADQ2.5/Gamma 2 Gliadin Peptide Complex, and HLA-DQ2.5/BC Hordein Peptide Complex

Expression and Purification of Recombinant HLA-DQ2.5/33mer Gliadin Peptide Complex:

The sequences used for expression and purification are: HLA-DQA1*0501 (Protein Data Bank accession code 4OZG) and HLA-DQB1*0201 (Protein Data Bank accession code 4OZG), both of which have a CAMPATH-1H signal sequence: MGWSCIILFLVATATGVHS (SEQ ID NO: 37). HLA-DQA1*0501 has C47S mutation, GGGG linker (SEQ ID NO: 38) and c-fos leucine zipper sequence (PNAS, 1998 Sep. 29; 95(20): 11828-33) and a Flag-tag on the C-terminus of HLA-DQA1*0501. HLADQB1*0201 has 33-mer gliadin peptide sequence: LQLQPFPQPELPYPQPELPYPQPELPYPQPQPF (SEQ ID NO: 39), and factor X cleavage linker (Acta Crystallogr Sect F Struct Biol Cryst Commun. 2007 Dec. 1; 63(Pt 12): 1021-1025) on the N-terminus of HLA-DQB1*0201, GGGGG linker (SEQ ID NO: 40) and c-jun leucine zipper sequence (PNAS, 1998 Sep. 29; 95(20): 11828-33), GGGGG linker (SEQ ID NO: 40), and BAP sequence (BMC Biotechnol. 2008; 8: 41), 8× His-tag on the C-terminus of HLA-DQB1*0201. A recombinant HLA-DQ2.5/33mer gliadin peptide complex was expressed transiently using FreeStyle293-F cell line (Thermo Fisher). Conditioned media expressing the HLA-DQ2.5/33mer gliadin peptide complex was incubated with an immobilized metal affinity chromatography (IMAC) resin, followed by elution with imidazole. Fractions containing the HLADQ2.5/33mer gliadin peptide complex were collected and subsequently subjected to a Superdex 200 gel filtration column (GE healthcare) equilibrated with 1× PBS. Fractions containing the HLA-DQ2.5/33mer gliadin peptide complex were then pooled and stored at −80 degrees Celsius (C). The purified HLA-DQ2.5/33mer gliadin peptide complex was biotinylated using BirA (Avidity).

Expression and Purification of Recombinant HLA-DQ8/Gliadin Peptide Complex:

The sequences used for expression and purification are: HLA-DQA1*0301 (Protein Data Bank accession code 4GG6) and HLA-DQB1*0302 (Protein Data Bank accession

code 4GG6), both of which have a CAMPATH-1H signal sequence: MGWSCIILFLVATATGVHS (SEQ ID NO: 37). HLA-DQA1*0301 has SSADLVPRGGGG linker (SEQ ID NO: 41) and c-fos leucine zipper sequence (PNAS, 1998 Sep. 29; 95(20): 11828-33) and a Flag-tag on the C-terminus of HLA-DQA1*0301. HLA-DQB1*0302 has gliadin peptide sequence: QQYPSGEGSFQPSQENPQ (SEQ ID NO: 42), and factor X cleavage linker (Acta Crystallogr Sect F Struct Biol Cryst Commun. 2007 Dec. 1; 63(Pt 12): 1021-1025) on the N-terminus of HLA-DQB1*0302, SSADLVPRGGGGG linker (SEQ ID NO: 43) and c-jun leucine zipper sequence (PNAS, 1998 Sep. 29; 95(20): 11828-33), GGGGG linker (SEQ ID NO: 40), and BAP sequence (BMC Biotechnol. 2008; 8: 41), 8× His-tag on the C-terminus of HLADQB1*0302. A recombinant HLA-DQ8/gliadin peptide was expressed transiently using FreeStyle293-F cell line. Conditioned media expressing the HLADQ8/gliadin peptide complex was incubated with an IMAC resin, followed by elution with imidazole. Fractions containing the HLA-DQ8/gliadin peptide complex were collected and subsequently subjected to a Superdex 200 gel filtration column equilibrated with 1×PBS. Fractions containing the HLA-DQ8/gliadin peptide complex were then pooled and stored at −80 degrees C.

Expression and Purification of Recombinant HLA-DQ5.1/DBY Peptide Complex:

The sequences used for expression and purification are: HLA-DQA1*0101 (IMGT/HLA accession No. HLA00601) and HLA-DQB1*0501 (IMGT/HLA accession No. HLA00638), both of which have a CAMPATH-1H signal sequence: MGWSCIILFLVATATGVHS (SEQ ID NO: 37). HLA-DQA1*0101 has C30Y mutation. HLA-DQA1*0101 has SSADLVPRGGGG linker (SEQ ID NO: 41) and cfos leucine zipper sequence (PNAS, 1998 Sep. 29; 95(20): 11828-33) and a Flag-tag on the C-terminus of HLA-DQA1*0101. HLA-DQB1*0501 has DBY peptide sequence: ATGSNCPPHIENFSDIDMGE (SEQ ID NO: 44), and factor X cleavage linker (Acta Crystallogr Sect F Struct Biol Cryst Commun. 2007 Dec. 1; 63(Pt 12): 1021-1025) on the N-terminus of HLA-DQB1*0501, SSADLVPRGGGGG linker (SEQ ID NO: 43) and c-jun leucine zipper sequence (PNAS, 1998 Sep. 29; 95(20): 11828-33), GGGGG linker (SEQ ID NO: 40), and BAP sequence (BMC Biotechnol. 2008; 8: 41), 8× His tag on the C-terminus of HLA-DQB1*0501. A recombinant HLA-DQ5.1/DBY peptide complex was expressed transiently using FreeStyle293-F cell line. Conditioned media expressing the HLA-DQ5.1/DBY peptide complex was incubated with an IMAC resin, followed by elution with imidazole. Fractions containing the HLA-DQ5.1/DBY peptide complex were collected and subsequently subjected to a Superdex 200 gel filtration column equilibrated with 1×PBS. Fractions containing the HLA-DQ5.1/DBY peptide complex were then pooled and stored at −80 degrees C. The purified HLA-DQ5.1/DBY peptide was biotinylated using BirA.

Expression and Purification of Recombinant HLA-DQ2.2/CLIP Peptide Complex:

The sequences used for expression and purification are: HLA-DQA1*0201 (IMGT/HLA accession No. HLA00607) and HLA-DQB1*0202 (IMGT/HLA accession No. HLA00623), both of which have a CAMPATH-1H signal sequence: MGWSCIILFLVATATGVHS (SEQ ID NO: 37). HLA-DQA1*0201 has SSADLVPRGGGG linker (SEQ ID NO: 41) and c-fos leucine zipper sequence (PNAS, 1998 Sep. 29; 95(20): 11828-33) and a Flag-tag on the C-terminus of HLADQA1*0201. HLA-DQB1*0202 has CLIP peptide sequence: KLPKPPKPVSKMRMATPLLMQALPMGALP (SEQ ID NO: 45), and factor X cleavage linker (Acta Crystallogr Sect F Struct Biol Cryst Commun. 2007 Dec. 1; 63(Pt 12): 1021-1025) on the N-terminus of HLA-DQB1*0202, SSADLVPRGGGGG linker (SEQ ID NO: 43) and c-jun leucine zipper sequence (PNAS, 1998 Sep. 29; 95(20): 11828-33), GGGGG linker (SEQ ID NO: 40), and BAP sequence (BMC Biotechnol. 2008; 8: 41), 8× His tag on the C-terminus of HLA-DQB1*0202. A recombinant HLA-DQ2.2/CLIP peptide complex was expressed transiently using FreeStyle293-F cell line. Conditioned media expressing the HLA-DQ2.2/CLIP peptide complex was incubated with an IMAC resin, followed by elution with imidazole. Fractions containing the HLA-DQ2.2/CLIP peptide complex were collected and subsequently subjected to a Superdex 200 gel filtration column equilibrated with 1×PBS. Fractions containing the HLA-DQ2.2/CLIP peptide complex were then pooled and stored at −80 degrees C.

Expression and Purification of Recombinant HLA-DQ7.5/CLIP Peptide Complex:

The sequences used for expression and purification are: HLA-DQA1*0505 (IMGT/HLA accession No. HLA00619) and HLA-DQB1*0301 (IMGT/HLA accession No. HLA00625), both of which have a CAMPATH-1H signal sequence: MGWSCIILFLVATATGVHS (SEQ ID NO: 37). HLA-DQA1*0505 has C66S mutation. HLA-DQA1*0505 has SSADLVPRGGGG linker (SEQ ID NO: 41) and cfos leucine zipper sequence (PNAS, 1998 Sep. 29; 95(20): 11828-33) and a Flag-tag on the C-terminus of HLA-DQA1*0505. HLA-DQB1*0301 has CLIP peptide sequence: KLPKPPKPVSKMRMATPLLMQALPMGALP (SEQ ID NO: 45), and factor X cleavage linker (Acta Crystallogr Sect F Struct Biol Cryst Commun. 2007 Dec. 1; 63(Pt 12): 1021-1025) on the N-terminus of HLA-DQB1*0301, SSADLVPRGGGGG linker (SEQ ID NO: 43) and c-jun leucine zipper sequence (PNAS, 1998 Sep. 29; 95(20): 11828-33), GGGGG linker (SEQ ID NO: 40), and BAP sequence (BMC Biotechnol. 2008; 8: 41), 8× His-tag on the C-terminus of HLA-DQB1*0301. A recombinant HLA-DQ7.5/CLIP peptide complex was expressed transiently using FreeStyle293-F cell line. Conditioned media expressing the HLA-DQ7.5/CLIP peptide complex was incubated with an IMAC resin, followed by elution with imidazole. Fractions containing the HLA-DQ7.5/CLIP peptide complex were collected and subsequently subjected to a Superdex 200 gel filtration column equilibrated with 1×PBS. Fractions containing the HLA-DQ7.5/CLIP peptide complex were then pooled and stored at −80 degrees C.

Expression and Purification of Recombinant HLA-DQ2.5/Gamma 2 Gliadin Peptide Complex:

The sequences used for expression and purification are: HLA-DQA1*0501 (Protein Data Bank accession code 4OZG) and HLA-DQB1*0201 (Protein Data Bank accession code 4OZG), both of which have a CAMPATH-1H signal sequence: MGWSCIILFLVATATGVHS (SEQ ID NO: 37). HLA-DQA1*0501 has C47S mutation, 3C protease cleavage linker: LEVLFQGP (SEQ ID NO: 46) and GGGG linker (SEQ ID NO: 38) and c-fos leucine zipper sequence (PNAS, 1998 Sep. 29; 95(20): 11828-33) and a Flag-tag on the C-terminus of HLA-DQA1*0501. HLADQB1*0201 has gamma 2 gliadin peptide sequence: IIQPEQPAQLP (SEQ ID NO: 47), and factor X cleavage linker (Acta Crystallogr Sect F Struct Biol Cryst Commun. 2007 Dec. 1; 63(Pt 12): 1021-1025) on the N-terminus of HLA-DQB1*0201, 3C protease cleavage linker: LEVLFQGP (SEQ ID NO: 46) and c-jun leucine zipper sequence (PNAS, 1998 Sep. 29; 95(20): 11828-33), GGGGG linker (SEQ ID NO: 40), and BAP sequence (BMC Biotechnol. 2008; 8: 41), 8× His-tag on the C-terminus of HLA-DQB1*0201. A recombinant HLA-DQ2.5/gamma 2 gliadin peptide complex was expressed transiently using FreeStyle293-F cell line. Conditioned media expressing the HLA-DQ2.5/gamma 2 gliadin peptide complex was incubated with an IMAC resin, followed by elution with imidazole. Fractions containing the HLA-DQ2.5/gamma 2 gliadin peptide complex were collected and subsequently subjected to a Superdex 200 gel filtration column equilibrated with 1×PBS. Fractions containing the HLA-DQ2.5/gamma 2 gliadin peptide complex were then pooled and stored at −80 degrees C.

Expression and Purification of Recombinant HLA-DQ2.5/BC Hordein Peptide Complex:

The sequences used for expression and purification are: HLA-DQA1*0501 (Protein Data Bank accession code 4OZG) and HLA-DQB1*0201 (Protein Data Bank accession code 4OZG), both of which have a CAMPATH-1H signal sequence: MGWSCIILFLVATATGVHS (SEQ ID NO: 37). HLA-DQA1*0501 has C47S mutation, 3C protease cleavage linker: LEVLFQGP (SEQ ID NO: 46) and GGGG linker (SEQ ID NO: 38) and c-fos leucine zipper sequence (PNAS, 1998 Sep. 29; 95(20): 11828-33) and a Flag-tag on the C-terminus of HLA-DQA1*0501. HLADQB1*0201 has BC Hordein peptide sequence: EPEQPIPEQPQPYPQQP (SEQ ID NO: 48), and factor X cleavage linker (Acta Crystallogr Sect F Struct Biol Cryst Commun. 2007 Dec. 1; 63(Pt 12): 1021-1025) on the N-terminus of HLA-DQB1*0201, 3C protease cleavage linker: LEVLFQGP (SEQ ID NO: 46) and c-jun leucine zipper sequence (PNAS, 1998 Sep. 29; 95(20): 11828-33), GGGGG linker (SEQ ID NO: 40), and BAP sequence (BMC Biotechnol. 2008; 8: 41), 8× His-tag on the C-terminus of HLA-DQB1*0201. A recombinant HLA-DQ2.5/BC Hordein peptide complex was expressed transiently using FreeStyle293-F cell line. Conditioned media expressing the HLA-DQ2.5/BC Hordein peptide complex was incubated with an IMAC resin, followed by elution with imidazole. Fractions containing the HLA-DQ2.5/BC Hordein peptide complex were collected and subsequently subjected to a Superdex 200 gel filtration column equilibrated with 1×PBS. Fractions containing the HLA-DQ2.5/BC Hordein peptide complex were then pooled and stored at −80 degrees C.

### Example 2

2.1 Establishment of D2 TCR-Expressing J.RT3-T3.5 Cell Lines

D2 TCR alpha chain cDNA (SEQ ID NO: 97) was inserted into the expression vector pCXND3 (WO2008/156083). D2 TCR beta chain cDNA (SEQ ID NO: 49) was inserted into the expression vector pCXZD1 (US2009/0324589). The linearized D2 TCR alpha chain—pCXND3 and D2 TCR beta chain—pCXZD1 (1500 ng each) were simultaneously introduced into J.RT3-T3.5 cell line by electroporation (LONZA, 4D-Nucleofector X). Transfected cells were then cultured in media containing Geneticin and Zeocin, after which sorting was performed to obtain a high-expressing cell population using AriaIII (Becton Dickinson). Single cell cloning was then performed to obtain cells that highly expressed the desired D2 TCR molecule.

2.2 Establishment of Ba/F3 Cell Lines Expressing HLA-DQ2.5, HLA-DQ2.2, HLADQ7.5, HLA-DQ8, HLA-DQ5.1, HLA-DQ6.3, HLA-DQ7.3, HLA-DR, and HLA-DP

HLA-DQA1*0501 cDNA (IMGT/HLA accession No. HLA00613), HLADQA1*0201 cDNA (IMGT/HLA accession No. HLA00607), HLA-DQA1*0505 cDNA (IMGT/HLA accession No. HLA00619), HLA-DQA1*0301 cDNA (IMGT/HLA accession No. HLA00608), HLA-DQA1*0101 cDNA (IMGT/HLA accession No. HLA00601), HLA-DQA1*0103 cDNA (IMGT/HLA accession No. HLA00604), HLA-DQA1*0303 cDNA (IMGT/HLA accession No. HLA00611), HLA-DRA1*0101 cDNA (GenBank accession No. NM_019111.4), or HLADPA1*0103 cDNA (IMGT/HLA accession No. HLA00499), was inserted into the expression vector pCXND3 (WO2008/156083) HLA-DQB1*0201 cDNA (IMGT/HLA accession No. HLA00622), HLADQB1*0202 cDNA (IMGT/HLA accession No. HLA00623), HLA-DQB1*0301 cDNA (IMGT/HLA accession No. HLA00625), HLA-DQB1*0302 cDNA (IMGT/HLA accession No. HLA00627), HLA-DQB1*0501 cDNA (IMGT/HLA accession No. HLA00638), HLA-DQB1*0603 cDNA (IMGT/HLA accession No. HLA00647), HLA-DRB1*0301 cDNA (IMGT/HLA accession No. HLA00671), or HLA-DPB1*0401 cDNA (IMGT/HLA accession No. HLA00521) was inserted into the expression vector pCXZD1 (US/20090324589).

Each of the linearized HLA-DQA1*0501-pCXND3 and HLADQB1*0201-pCXZD1, and each of the linearized HLA-DQA1*0201-pCXND3 and HLA-DQB1*0202-pCXZD1,

### HLA-DQA1*0505-pCXND3 and HLADQB1*0301-pCXZD1,

### HLA-DQA1*0301-pCXND3 and HLADQB1*0302-pCXZD1,

### HLA-DQA1*0101-pCXND3 and HLADQB1*0501-pCXZD1,

### HLA-DQA1*0103-pCXND3 and HLADQB1*0603-pCXZD1,

### HLA-DQA1*0303-pCXND3 and HLADQB1*0301-pCXZD1,

### HLA-DRA1*0101-pCXND3 and HLADRB1*0301-pCXZD1,

### HLA-DPA1*0103-pCXND3 and HLA-DPB1*0401-pCXZD1,

were simultaneously introduced into mouse IL-3-dependent pro-B cell-derived cell line Ba/F3 by electroporation (LONZA, 4D-Nucleofector X). Transfected cells were then cultured in media containing Geneticin and Zeocin. Cultured and expanded cell was then checked for the expression of HLA molecule and high expression of HLA was confirmed. This was performed to obtain cells that highly expressed the desired HLA molecules. Each cell lines established were named: Ba/F3-HLA-DQ2.5 (HLA-DQA1*0501, HLADQB1*0201), Ba/F3-HLA-DQ2.2 (HLA-DQA1*0201, HLA-DQB1*0202), Ba/F3-HLA-DQ7.5 (HLA-DQA1*0505, HLA-DQB1*0301), Ba/F3-HLA-DQ8 (HLA-DQA1*0301, HLADQB1*0302), Ba/F3-HLA-DQ5.1 (HLA-DQA1*0101, HLA-DQB1*0501), Ba/F3-HLA-DQ6.3 (HLA-DQA1*0103, HLA-DQB1*0603), Ba/F3-HLA-DQ7.3 (HLA-DQA1*0303, HLADQB1*0301), Ba/F3-HLA-DR (HLA-DRA1*0101, HLA-DRB1*0301), and Ba/F3-HLA-DP (HLA-DPA1*0103, HLA-DPB1*0401).

2.3 Establishment of Ba/F3 Cell Lines Expressing HLA-DQ2.5/CLIP Peptide, HLA-DQ2.5/Hepatitis B Virus Peptide, HLA-DQ2.5/Salmonella Peptide, HLADQ2.5/Thyroperoxidase Peptide, HLA-DQ2.5/Mycobacterium bovis Peptide, HLADQ2.5/Alpha 1 Gliadin Peptide, HLA-DQ2.5/Alpha 2 Gliadin Peptide, HLA-DQ2.5/Gamma 1 Gliadin Peptide, HLA-DQ2.5/Gamma 2 Gliadin Peptide, HLA-DQ2.5/Omega 1 Gliadin Peptide, HLA-DQ2.5/Omega 2 Gliadin Peptide, HLA-DQ2.5/BC Hordein Peptide, HLA-DQ2.5/Alpha 3 Gliadin Peptide, HLA-DQ2.5/Alpha 1b Gliadin Peptide, HLA-DQ2.5/Gamma 4b Gliadin Peptide, HLA-DQ2.5/Avenin 1 Peptide, HLA-DQ2.5/Avenin 2 Peptide, HLA-DQ2.5/Avenin 3 Peptide, HLA-DQ2.5/Hordein 1 Peptide, HLA-DQ2.5/Hordein 2 Peptide, HLA-DQ2.5/Secalin 1 Peptide, HLADQ2.5/Secalin 2 Peptide, HLA-DQ2.5/14mer 1 Peptide, HLA-DQ2.5/33mer Gliadin Peptide, HLA-DQ2.5/26mer Gliadin Peptide

HLA-DQA1*0501 cDNA (IMGT/HLA accession No. HLA00613) was inserted into the expression vector pCXND3 (WO2008/156083). HLA-DQB1*0201 cDNA (IMGT/HLA accession No. HLA00622) was inserted into the expression vector pCXZD1 (US/20090324589). HLA-DQB1*0201 for the HLA-DQ2.5/each peptide complex has each peptide sequence and factor X cleavage linker: (Acta Crystallogr Sect F Struct Biol Cryst Commun. 2007 Dec. 1; 63(Pt 12): 1021-1025) on the N-terminus of HLA-DQB1*0201. In particular, each peptide sequence was as follows: KLPKPPKPVSKMRMATPLLMQALPMGALP (SEQ ID NO: 45) was used for the CLIP peptide sequence, PDRVHFASPLHVAWR (SEQ ID NO: 50) was used for the Hepatits B virus peptide sequence, MMAWRMMRY (SEQ ID NO: 51) was used for the Salmonella peptide sequence, YIDVWLGGLAENFLPY (SEQ ID NO: 52) was used for the Thyroperoxidase peptide sequence, KPLLIIAEDVEGEY (SEQ ID NO: 53) was used for the Mycobacterium bovis peptide sequence, QPFPQPELPYP (SEQ ID NO: 54) was used for the alpha 1 gliadin peptide sequence, FPQPELPYPQP (SEQ ID NO: 55) was used for the alpha 2 gliadin peptide sequence, QPQQSFPEQQQ (SEQ ID NO: 56) was used for the gamma 1 gliadin peptide sequence, GIIQPEQPAQLP (SEQ ID NO: 57) was used for the gamma 2 gliadin peptide sequence, QPFPQPEQPFP (SEQ ID NO: 58) was used for the omega 1 gliadin peptide sequence, FPQPEQPFPWQ (SEQ ID NO: 59) was used for the omega 2 gliadin peptide sequence, PQQPIPEQPQPYPQQP (SEQ ID NO: 60) was used for the BC hordein peptide sequence, PFRPEQPYPQP (SEQ ID NO: 61) was used for the alpha 3 gliadin peptide sequence, LPYPQPELPYP (SEQ ID NO: 62) was used for the alpha 1b gliadin peptide sequence, FPQPEQEFPQP (SEQ ID NO: 63) was used for the gamma 4b gliadin peptide sequence, QPYPEQEEPFV (SEQ ID NO: 64) was used for the avenin 1 peptide sequence, QPYPEQEQPFV (SEQ ID NO: 65) was used for the avenin 2 peptide sequence, QPYPEQEQPIL (SEQ ID NO: 66) was used for the avenin 3 peptide sequence, PQQPFPQPEQPFRQ (SEQ ID NO: 67) was used for the hordein 1 peptide sequence, QEFPQPEQPFPQQP (SEQ ID NO: 68) was used for the hordein 2 peptide sequence, PEQPFPQPEQPFPQ (SEQ ID NO: 69) was used for the secalin 1 peptide sequence, QPFPQPEQPFPQSQ (SEQ ID NO: 70) was used for the secalin 2 peptide sequence, PQQQTLQPEQPAQLP (SEQ ID NO: 71) was used for the 14mer 1 peptide sequence, LQLQPFPQPELPYPQPELPYPQPELPYPQPQPF (SEQ ID NO: 39) was used for the 33mer gliadin peptide sequence, FLQPEQPFPEQPEQPYPE-QPEQPFPQ (SEQ ID NO: 72) was used for the 26mer gliadin peptide sequence. Each of the linearized HLA-DQA1*0501-pCXND3 and HLA-DQB1*0201/each peptide-pCXZD1 were simultaneously introduced into mouse IL-3-dependent pro-B cell-derived cell line Ba/F3 by electroporation (LONZA, 4D-Nucleofector X). Transfected cells were then cultured in media containing Geneticin and Zeocin. Cultured and expanded cell was then checked for the expression of HLA-DQ2.5 molecule and high expression of HLA-DQ2.5 was confirmed. Each cell lines established were named: Ba/F3-HLA-DQ2.5/CLIP (HLA-DQA1*0501, HLA-DQB1*0201 for HLADQ2.5/CLIP peptide), Ba/F3-HLA-DQ2.5/HBV (HLA-DQA1*0501, HLA-DQB1*0201 for HLADQ2.5/Hepatitis B virus peptide), Ba/F3-HLA-DQ2.5/Salmonella (HLA-DQA1*0501, HLA-DQB1*0201 for HLADQ2.5/Salmonella peptide), Ba/F3-HLA-DQ2.5/TPO (HLA-DQA1*0501, HLA-DQB1*0201 for HLADQ2.5/Thyroperoxidase peptide), Ba/F3-HLA-DQ2.5/M. bovis (HLA-DQA1*0501, HLA-DQB1*0201 for HLADQ2.5/Mycobacterium bovis peptide), Ba/F3-HLA-DQ2.5/alpha 1 gliadin (HLA-DQA1*0501, HLA-DQB1*0201 for HLADQ2.5/alpha 1 gliadin peptide), Ba/F3-HLA-DQ2.5/alpha 2 gliadin (HLA-DQA1*0501, HLA-DQB1*0201 for HLADQ2.5/alpha 2 gliadin peptide), Ba/F3-HLA-DQ2.5/gamma 1 gliadin (HLA-DQA1*0501, HLA-DQB1*0201 for HLADQ2.5/gamma 1 gliadin peptide), Ba/F3-HLA-DQ2.5/gamma 2 gliadin (HLA-DQA1*0501, HLA-DQB1*0201 for HLADQ2.5/gamma 2 gliadin peptide), Ba/F3-HLA-DQ2.5/omega 1 gliadin (HLA-DQA1*0501, HLA-DQB1*0201 for HLADQ2.5/omega 1 gliadin peptide), Ba/F3-HLA-DQ2.5/omega 2 gliadin (HLA-DQA1*0501, HLA-DQB1*0201 for HLA-DQ2.5/omega 2 gliadin peptide), Ba/F3-HLA-DQ2.5/BC hordein (HLA-DQA1*0501, HLA-DQB1*0201 for HLADQ2.5/BC hordein peptide), Ba/F3-HLA-DQ2.5/alpha 3 gliadin (HLA-DQA1*0501, HLA-DQB1*0201 for HLADQ2.5/alpha 3 gliadin peptide), Ba/F3-HLA-DQ2.5/alpha 1b gliadin (HLA-DQA1*0501, HLA-DQB1*0201 for HLADQ2.5/alpha 1b gliadin peptide), Ba/F3-HLA-DQ2.5/gamma 4b gliadin (HLA-DQA1*0501, HLA-DQB1*0201 for HLADQ2.5/gamma 4b gliadin peptide), Ba/F3-HLA-DQ2.5/avenin 1 (HLA-DQA1*0501, HLA-DQB1*0201 for HLA-DQ2.5/avenin 1 peptide), Ba/F3-HLA-DQ2.5/avenin 2 (HLA-DQA1*0501, HLA-DQB1*0201 for HLA-DQ2.5/avenin 2 peptide), Ba/F3-HLA-DQ2.5/avenin 3 (HLA-DQA1*0501, HLA-DQB1*0201 for HLA-DQ2.5/avenin 3 peptide), Ba/F3-HLA-DQ2.5/hordein 1 (HLA-DQA1*0501, HLA-DQB1*0201 for HLA-DQ2.5/hordein 1 peptide), Ba/F3-HLA-DQ2.5/hordein 2 (HLA-DQA1*0501, HLA-DQB1*0201 for HLA-DQ2.5/hordein 2 peptide), Ba/F3-HLA-DQ2.5/secalin 1 (HLA-DQA1*0501, HLA-DQB1*0201 for HLA-DQ2.5/secalin 1 peptide), Ba/F3-HLA-DQ2.5/secalin 2 (HLA-DQA1*0501, HLA-DQB1*0201 for HLA-DQ2.5/secalin 2 peptide), Ba/F3-HLA-DQ2.5/14mer 1 (HLA-DQA1*0501, HLA-DQB1*0201 for HLADQ2.5/14mer 1 peptide), Ba/F3-HLA-DQ2.5/33mer gliadin (HLA-DQA1*0501, HLA-DQB1*0201 for HLA-DQ2.5/33mer gliadin peptide), and Ba/F3-HLA-DQ2.5/26mer gliadin (HLA-DQA1*0501, HLA-DQB1*0201 for HLADQ2.5/26mer gliadin peptide).

### Example 3

Generation of Anti-DQ2.5 Antibodies

Anti-DQ2.5 antibodies were prepared, selected and assayed as follows: NZW rabbits were immunized intradermally with the HLA-DQ2.5/33mer gliadin peptide complex. Four repeated doses were given over a 2-month period followed by blood and spleen collection. For B-cell selection, a biotinylated HLA-DQ5.1/DBY peptide complex, biotinylated HLA-DQ8/gliadin peptide complex, and Alexa Fluor 488-labeled HLA-DQ2.5/33mer gliadin peptide complex were prepared. B-cells that can bind to HLA-DQ2.5 but not HLA-DQ5.1 or HLA-DQ8 were stained with the labeled proteins described above, sorted using a cell sorter and then plated and cultured according to the procedure described in WO2016098356A1. After cultivation, the B cell culture supernatants were collected for further analysis and the B-cell pellets were cryopreserved.

Specific binding to the HLA-DQ2.5/33mer gliadin peptide complex was evaluated and non cross-reactivity to the HLA-DQ5.1/DBY peptide complex and the HLA-DQ8/gliadin peptide complex was confirmed by ELISA using the B cell culture supernatants. The results showed that 336 B cell lines exhibited specific binding to the HLA-DQ2.5/33mer gliadin peptide complex.
In order to evaluate the cross-reactivity to the HLA-DQ2.2/CLIP peptide complex and the HLA-DQ7.5/CLIP peptide complex, ELISA was conducted using the selected 336 B cell supernatants described above. In addition, neutralizing activity was checked by neutralizing assay using the selected 336 B cell supernatants.
The procedure of neutralizing assay was in accordance with the AlphaLISA neutralizing assay (HLA-DQ2.5/33mer gliadin peptide-D2 TCR) described below. B cells with high neutralizing activities were preferred and selected for cloning.
The RNAs of 180 B cell lines with desired binding specificities were purified from the cryopreserved cell pellets using the ZR-96 Quick-RNA kits (ZYMO RESEARCH, Cat No. R1053). These were named DQN0377-0464. DNAs encoding antibody heavy chain variable regions in the selected cell lines were amplified by reverse transcription PCR and recombined with a DNA encoding the F1332m heavy-chain constant region (SEQ ID NO: 73) (WO2018/155692). DNAs encoding antibody light-chain variable regions were also amplified by reverse transcription PCR and recombined with a DNA encoding the hk0MC light-chain constant region (SEQ ID NO: 74) (WO2018/155692). Cloned antibodies were expressed in Freestyle™ 293-F Cells (Invitrogen) and purified from culture supernatants. Through further evaluation described below, two clones (DQN0385ee, DQN0429cc) were selected based on binding ability, specificity and functionality. DQN0344xx (WO2019/069993) was also utilized. DQN0139bb (WO2018/155692) was used as an assay control. The sequence ID numbers of VH, VL, HCDRs and LCDRs of these antibodies are listed in Table 1 above. The sequences of FR1 to FR4 and CDR1 to CDR3 of the heavy and light chains of these antibodies are shown in Table 2.

TABLE 2

| | F R 1 | | | | | | | | | | | | | | | | | | | | | | | | | | | | | | C D R 1 | | | | | | |
|---|---|---|---|---|---|---|---|---|---|---|---|---|---|---|---|---|---|---|---|---|---|---|---|---|---|---|---|---|---|---|---|---|---|---|---|---|---|
| H chain | 0 | | | | | | | | | 1 | | | | | | | | | | 2 | | | | | | | | | | 3 | 3 | | | | | | |
| Name | 1 | 2 | 3 | 4 | 5 | 6 | 7 | 8 | 9 | 0 | 1 | 2 | 3 | 4 | 5 | 6 | 7 | 8 | 9 | 0 | 1 | 2 | 3 | 4 | 5 | 6 | 7 | 8 | 9 | 0 | 1 | 2 | 3 | 4 | 5 | a | b |
| DQN0385ee | Q | E | Q | L | E | E | S | G | G | D | L | V | K | P | G | A | S | L | T | L | T | C | T | A | S | G | F | S | F | Y | S | S | Y | W | I | C | — |
| DQN0429cc | Q | E | Q | L | V | E | S | G | G | G | L | V | Q | P | E | G | S | L | T | L | T | C | T | A | S | G | F | S | F | S | S | S | Y | Y | M | C | — |
| DQN0344x | Q | | S | L | E | E | S | G | G | G | L | V | K | P | G | G | T | L | T | L | T | C | T | A | S | G | F | S | F | S | S | S | Y | W | M | C | — |
| IC17 | Q | V | Q | L | Q | Q | S | G | P | Q | L | V | R | P | G | A | S | V | K | I | S | C | K | A | S | G | Y | S | F | T | S | Y | W | M | H | — | — |
| | | | | | F R 2 | | | | | | | | | | | | | | C D R 2 | | | | | | | | | | | | | | | | | | |
| H chain | | | | | 3 | | | | 4 | | | | | | | | | | 5 | | | | | | | | | | | | | 6 | | | | | |
| Name | | | | | 6 | 7 | 8 | 9 | 0 | 1 | 2 | 3 | 4 | 5 | 6 | 7 | 8 | 9 | 0 | 1 | 2 | a | b | c | 3 | 4 | 5 | 6 | 7 | 8 | 9 | 0 | 1 | 2 | 3 | 4 | 5 |
| DQN0385ee | | | | | W | V | R | Q | A | P | G | K | G | L | E | W | I | A | C | I | D | T | — | — | G | S | G | S | I | D | Y | A | S | W | V | N | G |
| DQN0429cc | | | | | W | V | R | Q | A | P | G | K | G | L | E | W | I | G | C | I | Y | T | — | — | G | S | G | S | T | D | Y | A | T | W | V | N | G |
| DQN0344xx | | | | | W | V | R | Q | A | P | G | K | G | L | E | W | V | A | C | V | Y | G | — | — | G | S | D | T | T | Y | Y | A | S | W | T | K | G |
| IC17 | | | | | W | V | N | Q | R | P | G | Q | G | L | E | W | I | G | M | I | D | P | — | — | S | Y | S | E | T | R | L | N | Q | K | F | K | D |
| | | | | | | F R 3 | | | | | | | | | | | | | | | | | | | | | | | | | | | | | | | |
| H chain | | | | | | 6 | | | | 7 | | | | | | | | | | 8 | | | | | | | | | | | | | 9 | | | | |
| Name | | | | | | 6 | 7 | 8 | 9 | 0 | 1 | 2 | 3 | 4 | 5 | 6 | 7 | 8 | 9 | 0 | 1 | 2 | a | b | c | 3 | 4 | 5 | 6 | 7 | 8 | 9 | 0 | 1 | 2 | 3 | 4 |
| DQN0385ee | | | | | | R | F | T | I | S | K | — | T | S | S | T | T | V | T | L | Q | M | T | S | L | T | V | A | D | T | A | T | Y | F | C | A | R |
| DQN0429cc | | | | | | R | F | T | I | S | K | — | T | S | S | T | T | V | T | L | Q | M | T | S | L | T | V | A | D | T | A | T | Y | F | C | A | R |
| DQN0344xx | | | | | | R | F | T | I | S | K | G | S | S | T | | T | V | A | L | Q | V | T | S | L | T | A | A | D | T | A | T | Y | F | C | A | R |
| IC17 | | | | | | K | A | T | L | T | V | D | K | S | S | S | T | A | Y | M | Q | L | S | S | P | T | S | E | D | S | A | V | Y | Y | C | A | L |
| | | | | | | | C D R 3 | | | | | | | | | | | | | | | | | | | | F R 4 | | | | | | | | | | |
| H chain | | | | | | | 9 | | | | | 1 | 0 | | | | | | | | | | | | | | | | | | | | | 1 | 1 | | |
| Name | | | | | | | 5 | 6 | 7 | 8 | 9 | 0 | a | b | c | d | e | f | g | h | i | j | k | l | 1 | 2 | 3 | 4 | 5 | 6 | 7 | 8 | 9 | 0 | 1 | 2 | 3 |
| DQN0385ee | | | | | | | D | I | G | I | D | Y | — | — | — | — | — | — | — | — | — | — | — | — | N | F | W | G | P | G | T | L | V | T | V | S | S |
| DQN0429cc | | | | | | | D | I | G | I | D | Y | — | — | — | — | — | — | — | — | — | — | — | — | N | F | W | G | P | G | T | L | V | T | V | S | S |
| DQN0344xx | | | | | | | D | P | L | N | Y | Y | Y | Y | G | E | L | — | — | — | — | — | — | — | N | L | W | G | P | G | T | L | V | T | V | S | S |
| IC17 | | | | | | | Y | G | N | Y | F | — | — | — | — | — | — | — | — | — | — | — | — | — | D | Y | W | G | Q | G | T | T | L | T | V | S | S |
| | | | | | | | | | | | | | | | F R 1 | | | | | | | | | | | | | | | | | | | | | | |
| L chain | | | | | | | | | | | | | | | 0 | | | | | | | | | 1 | | | | | | | | | | 2 | | | |
| Name | | | | | | | | | | | | | | | 1 | 2 | 3 | 4 | 5 | 6 | 7 | 8 | 9 | 0 | 1 | 2 | 3 | 4 | 5 | 6 | 7 | 8 | 9 | 0 | 1 | 2 | 3 |
| DQN0385ee | | | | | | | | | | | | | | | A | F | E | L | T | Q | T | P | S | F | V | E | A | A | V | G | G | T | V | T | I | K | C |
| DQN0429cc | | | | | | | | | | | | | | | A | F | E | L | T | Q | T | P | S | F | V | E | A | R | V | G | G | T | V | T | I | K | C |
| DQN0344xx | | | | | | | | | | | | | | | A | V | V | L | T | Q | T | A | S | P | V | S | A | A | V | G | G | T | V | T | I | R | C |
| IC17 | | | | | | | | | | | | | | | D | I | Q | M | T | Q | S | S | S | S | F | S | V | S | L | G | D | R | V | T | I | T | C |
| | | | | | | C D R 1 | | | | | | | | | | | | | | | | | F R 2 | | | | | | | | | | | | | | |
| L chain | | | | | | 2 | | | | | | | | | | | | 3 | | | | | 3 | | | | | 4 | | | | | | | | | |
| Name | | | | | | 4 | 5 | 6 | 7 | a | b | c | d | e | f | 8 | 9 | 0 | 1 | 2 | 3 | 4 | 5 | 6 | 7 | 8 | 9 | 0 | 1 | 2 | 3 | 4 | 5 | 6 | 7 | 8 | 9 |
| DQN0385ee | | | | | | Q | A | T | Q | — | — | — | — | — | — | S | I | S | S | Y | L | N | W | Y | Q | Q | K | P | G | Q | P | P | K | L | L | I | Y |
| DQN0429cc | | | | | | Q | A | T | Q | — | — | — | — | — | — | S | I | S | S | Y | L | N | W | Y | Q | Q | K | P | G | Q | P | P | K | L | L | I | Y |
| DQN0344xx | | | | | | Q | A | T | E | — | — | — | — | — | — | N | I | Y | S | G | L | A | W | Y | Q | Q | K | P | G | Q | P | P | K | V | L | I | Y |
| IC17 | | | | | | K | A | S | E | — | — | — | — | — | — | D | I | Y | N | R | L | A | W | Y | Q | Q | K | P | G | N | A | P | R | L | L | I | S |
| | | | | | | | | | | | | | | | | | | | | | | | | | | | | | | | C D R 2 | | | | | | |
| L chain | | | | | | | | | | | | | | | | | | | | | | | | | | | | | | | 5 | | | | | | |
| Name | | | | | | | | | | | | | | | | | | | | | | | | | | | | | | | 0 | 1 | 2 | 3 | 4 | 5 | 6 |
| DQN0385ee | | | | | | | | | | | | | | | | | | | | | | | | | | | | | | | Y | A | S | T | L | A | S |
| DQN0429cc | | | | | | | | | | | | | | | | | | | | | | | | | | | | | | | Y | A | S | T | L | A | S |
| DQN0344xx | | | | | | | | | | | | | | | | | | | | | | | | | | | | | | | Y | V | S | T | L | A | S |
| IC17 | | | | | | | | | | | | | | | | | | | | | | | | | | | | | | | G | A | T | S | L | E | T |
| | | | | | | F R 3 | | | | | | | | | | | | | | | | | | | | | | | | | | | | | | | |
| L chain | | | | | | 5 | | | 6 | | | | | | | | | | 7 | | | | | | | | | | 8 | | | | | | | | |
| Name | | | | | | 7 | 8 | 9 | 0 | 1 | 2 | 3 | 4 | 5 | 6 | 7 | 8 | 9 | 0 | 1 | 2 | 3 | 4 | 5 | 6 | 7 | 8 | 9 | 0 | 1 | 2 | 3 | 4 | 5 | 6 | 7 | 8 |
| DQN0385ee | | | | | | G | V | P | S | R | F | K | G | S | G | S | G | T | E | F | S | L | T | I | S | G | V | E | C | A | D | A | A | T | Y | Y | C |
| DQN0429cc | | | | | | G | V | P | S | R | F | K | G | S | G | S | G | T | E | F | T | L | T | I | S | G | V | E | C | A | D | A | A | T | Y | Y | C |
| DQN0344xx | | | | | | G | V | P | S | R | F | K | G | S | G | S | G | T | E | Y | T | L | T | I | S | G | V | Q | C | D | D | A | A | T | Y | Y | C |
| IC17 | | | | | | G | V | P | S | R | F | S | G | S | G | S | G | K | D | Y | T | L | S | I | T | S | L | Q | T | E | D | V | A | T | Y | Y | C |
| | | | | | | | | | | | | | C D R 3 | | | | | | | | | | | | | | | F R 4 | | | | | | | | | |
| L chain | | | | | | | | | | | | | | 9 | | | | | | | | | | | | | | | | 1 | 0 | | | | | | |
| Name | | | | | | | | | | | | | 9 | 0 | 1 | 2 | 3 | 4 | 5 | a | b | c | d | e | f | 6 | 7 | 8 | 9 | 0 | 1 | 2 | 3 | 4 | 5 | 6 | 7 |
| DQN0385ee | | | | | | | | | | | | | H | Y | G | I | S | Y | V | S | — | — | — | — | — | — | — | F | G | G | G | T | K | V | E | I | K |
| DQN0492cc | | | | | | | | | | | | | H | Y | G | I | S | Y | V | S | — | — | — | — | — | — | — | F | G | G | G | T | K | V | E | I | K |
| DQN0344xx | | | | | | | | | | | | | Q | T | Y | H | D | I | S | N | — | — | — | — | — | V | T | F | G | G | G | T | K | V | E | I | K |
| IC17 | | | | | | | | | | | | | Q | Q | Y | W | S | T | P | — | — | — | — | — | — | Y | T | F | G | G | G | T | K | L | E | V | K |

### Example 4

### Generation of Bispecific Antibodies:

Bispecific antibodies which demonstrate cross-reactive binding to the multiple HLADQ2.5/gluten peptide complex were generated. To generate bispecific antibodies, six multi-gluten peptide selective HLA-DQ2.5 bivalent antibodies (DQN0344Hx-SG181.S3n, DQN0385He-SG181.S3n, DQN0429Hc-SG181.S3n, DQN0139Hb-SG181.S3n, p, DQN0385He-SG181.S3p, and DQN0429Hc-SG181.S3p) and one negative control antibody (IC17HdK-SG181.S3p) were used. SG181 is a Fc gamma receptor silenced Fc which attenuates Fc binding against Fc gamma receptors. cDNAs encoding the antibodies including the variable region and human IgG1 constant region were synthesized and cloned into the standard mammalian expression vector. Each of the bivalent antibodies was transiently transfected and expressed using an Expi293 Expression system (Thermo Fisher Scientific). Culture supernatants were harvested, and antibodies were purified from the supernatants using MabSelect SuRe pcc affinity chromatography (GE Healthcare) and subsequent gel permeate chromatography using Superdex200 (GE Healthcare). To generate the bispecific antibodies, purified seven bivalent antibodies were subjected to Fab arm exchanging technology (as described in WO2015/046467). Six bispecific antibodies were then generated and named DQN0344xx//IC17, DQN0385ee//IC17, DQN0429cc//IC17, DQN0344xx//DQN0385ee, DQN0344xx//DQN0429cc, and DQN0139bb//IC17, respectively. Summary and sequences of the bispecific antibodies are shown in Table 3. SG181.S3n (SEQ ID NO: 33) and SG181.S3p (SEQ ID NO: 34) are heavy chain constant regions sequences. k0MC (SEQ ID NO: 35) and SK1 (SEQ ID NO: 36) are light chain constant region sequences.
Summary and sequences of bispecific antibodies are shown in Table 3 below.

TABLE 3

| | | | Bispecific antibody name | | |
|---|---|---|---|---|---|
| | | | DQN0344xx//IC17 | DQN0385ee//IC17 | DQN0429cc//IC17 |
| | | | Arm A | | |
| | | | DQN0344Hx-SG181.S3n | DQN0385He-SG181.S3n | DQN0429Hc-SG181.S3n |
| | | | Arm B | | |
| | | | IC17HdK-SG181.S3p | IC17HdK-SG181.S3p | IC17HdK-SGl81.S3p |
| SEQ ID NO: | Arm A | HCDR1 | 10 | 2 | 6 |
| | | HCDR2 | 11 | 3 | 7 |
| | | HCDR3 | 12 | 4 | 8 |
| | | Heavy chain variable region | 9 | 1 | 5 |
| | | Heavy chain constant region | 33 | 33 | 33 |
| | | LCDR1 | 26 | 18 | 22 |
| | | LCDR2 | 27 | 19 | 23 |
| | | LCDR3 | 28 | 20 | 24 |
| | | Light chain variable region | 25 | 17 | 21 |
| | | Light chain constant region | 35 (k0MC) | 35 (k0MC) | 35 (k0MC) |
| | Arm B | HCDRl | 14 | 14 | 14 |
| | | HCDR2 | 15 | 15 | 15 |
| | | HCDR3 | 16 | 16 | 16 |
| | | Heavy chain variable region | 13 | 13 | 13 |
| | | Heavy chain constant region | 34 | 34 | 34 |
| | | LCDR1 | 30 | 30 | 30 |
| | | LCDR2 | 31 | 31 | 31 |
| | | LCDR3 | 32 | 32 | 32 |
| | | Light chain variable region | 29 | 29 | 29 |
| | | Light chain constant region | 36 (SK1) | 36 (SK1) | 36 (SK1) |

| | | | Bispecific antibody name | | |
|---|---|---|---|---|---|
| | | | DQN0344xx//DQN0385ee | DQN0344xx//DQN0429cc | DQN0139bb//IC17 |
| | | | Arm A | | |
| | | | DQN0344Hx-SGl81.S3n | DQN0344Hx-SGl81.S3n | DQN0139Hb-SG181.S3n |
| | | | Arm B | | |
| | | | DQN0385He-SG181.S3p | DQN0429Hc-SG181.S3p | IC17HdK-SG181.S3p |
| SEQ ID NO: | Arm A | HCDR1 | 10 | 10 | 86 |
| | | HCDR2 | 11 | 11 | 87 |
| | | HCDR3 | 12 | 12 | 88 |
| | | Heavy chain variable region | 9 | 9 | 85 |
| | | Heavy chain constant region | 33 | 33 | 33 |
| | | LCDR1 | 26 | 26 | 90 |
| | | LCDR2 | 27 | 27 | 91 |
| | | LCDR3 | 28 | 28 | 92 |
| | | Light chain variable region | 25 | 25 | 89 |
| | | Light chain constant region | 35 (k0MC) | 35 (k0MC) | 35 (k0MC) |
| | Arm B | HCDRl | 2 | 6 | 14 |
| | | HCDR2 | 3 | 7 | 15 |
| | | HCDR3 | 4 | 8 | 16 |
| | | Heavy chain variable region | 1 | 5 | 13 |
| | | Heavy chain constant region | 34 | 34 | 34 |
| | | LCDR1 | 18 | 22 | 30 |
| | | LCDR2 | 19 | 23 | 31 |
| | | LCDR3 | 20 | 24 | 32 |
| | | Light chain variable region | 17 | 21 | 29 |
| | | Light chain constant region | 35 (k0MC) | 35 (k0MC) | 36 (SK1) |

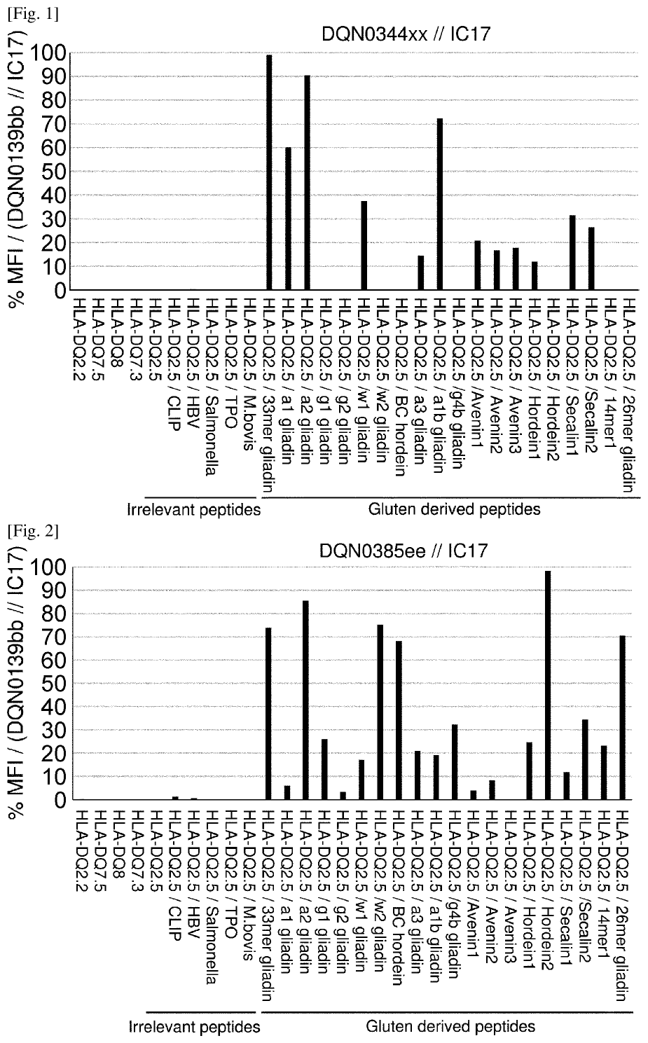

Binding analysis of the antibodies to HLA:
FIGS. 1 to 12 show the binding of the each of the anti-HLA-DQ antibodies to a panel of Ba/F3 cell lines expressing HLA-DQ in the form of a complex with several peptides as determined by FACS. The binding of anti-HLA-DQ antibodies to Ba/F3-HLA-DQ2.5, Ba/F3-HLA-DQ2.2, Ba/F3-HLA-DQ7.5, Ba/F3-HLA-DQ8, Ba/F3-HLA-DQ5.1, Ba/F3-HLA-DQ6.3, Ba/F3-HLA-DQ7.3, Ba/F3-HLA-DR, Ba/F3-HLA-DP, Ba/F3-HLA-DQ2.5/CLIP, Ba/F3-HLA-DQ2.5/HBV, Ba/F3-HLA-DQ2.5/Salmonella, Ba/F3-HLA-DQ2.5/TPO, Ba/F3-HLA-DQ2.5/M. bovis, Ba/F3-HLA-DQ2.5/alpha 1 gliadin, Ba/F3-HLA-DQ2.5/alpha 2 gliadin, Ba/F3-HLA-DQ2.5/gamma 1 gliadin, Ba/F3-HLA-DQ2.5/gamma 2 gliadin, Ba/F3-HLA-DQ2.5/omega 1 gliadin, Ba/F3-HLA-DQ2.5/omega 2 gliadin, Ba/F3-HLA-DQ2.5/BC hordein, Ba/F3-HLA-DQ2.5/alpha 3 gliadin, Ba/F3-HLA-DQ2.5/alpha 1b gliadin, Ba/F3-HLA-DQ2.5/gamma 4b gliadin, Ba/F3-HLA-DQ2.5/avenin 1, Ba/F3-HLA-DQ2.5/avenin 2, Ba/F3-HLA-DQ2.5/avenin 3, Ba/F3-HLA-DQ2.5/hordein 1, Ba/F3-HLA-DQ2.5/hordein 2, Ba/F3-HLA-DQ2.5/secalin 1, Ba/F3-HLA-DQ2.5/secalin 2, Ba/F3-HLA-DQ2.5/14mer 1, Ba/F3-HLA-DQ2.5/33mer gliadin, Ba/F3-HLA-DQ2.5/26mer gliadin, was tested. 5 microgram/mL of each of the anti-HLA-DQ antibodies was incubated with each cell line for 30 minutes at room temperature and washed with FACS buffer (2% FBS, 2 mM EDTA in PBS). Goat F(ab′)2 anti-Human IgG, Mouse ads-PE (Southern Biotech, Cat. 2043-09) was then added and incubated for 20 minutes at 4 degrees C., and this was washed with FACS buffer. Data acquisition was performed on LSRFortessa X-20 (Becton Dickinson), followed by analysis using the FlowJo software (Tree Star) and Microsoft Office Excel 2013. % MFI of bispecific antibodies was determined when taking a MFI value of IC17 as 0% and a MFI value of DQN0139bb/IC17 as 100%. % MFI of bivalent antibodies was determined when taking a MFI value of IC17 as 0% and a MFI value of DQN0139bb as 100%

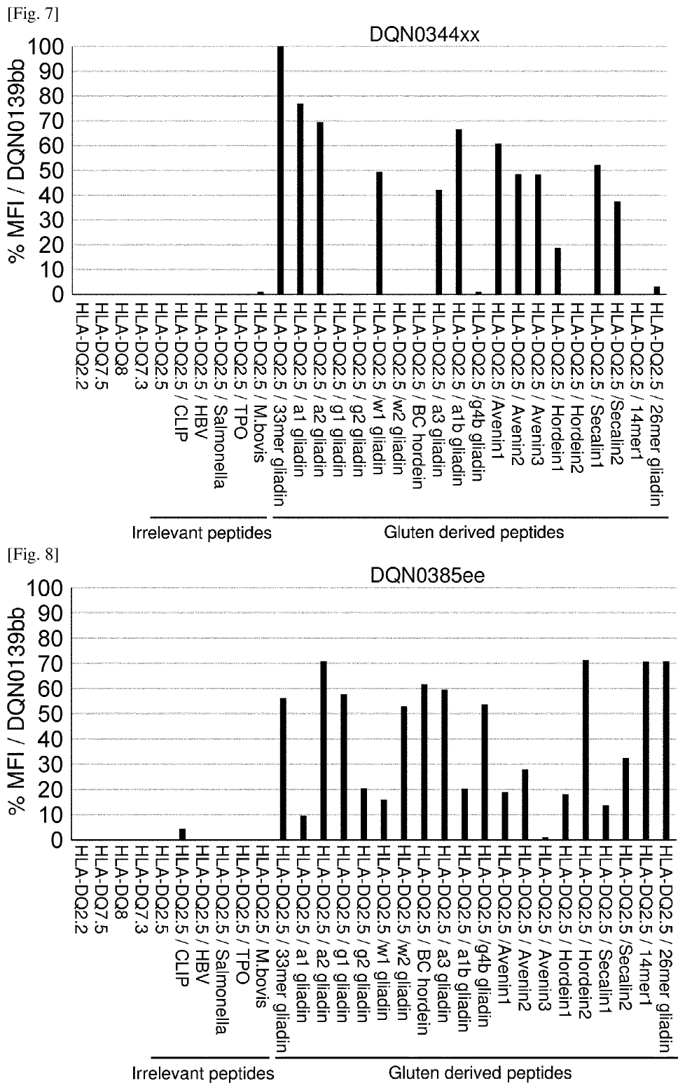

FIG. 1 and FIG. 7 shows that DQN0344xx and DQN0344xx//IC17 have binding activity to HLA-DQ2.5 only when it is in the form of a complex with gluten derived peptides, in particular 33mer gliadin peptide, alpha 1 gliadin peptide, alpha 2 gliadin peptide, omega 1 gliadin peptide, alpha 3 gliadin peptide, alpha 1b gliadin peptide, avenin 1 peptide, avenin 2 peptide, avenin 3 peptide, hordein 1 peptide, secalin 1 peptide, secalin 2 peptide. On the other hand, DQN0344xx and DQN0344xx//IC17 have substantially no binding activity to HLA-DQ2.5 when it is in the form of a complex with peptides which are irrelevant to gluten peptides.

FIG. 2 and FIG. 8 show that DQN0385ee and DQN0385ee//IC17 have binding activity to HLA-DQ2.5 only when it is in the form of a complex with 33mer gliadin peptide, alpha 1 gliadin peptide, alpha 2 gliadin peptide, gamma 1 gliadin peptide, gamma 2 gliadin peptide, omega 1 gliadin peptide, omega 2 gliadin peptide, BC hordein peptide, alpha 3 gliadin peptide, alpha 1b gliadin peptide, gamma 4b gliadin peptide, avenin 1 peptide, avenin 2 peptide, hordein 1 peptide, hordein 2 peptide, secalin 1 peptide, secalin 2 peptide, 14mer1 peptide, and 26mer gliadin peptide. On the other hand, DQN0385ee and DQN0385ee//IC17 have substantially no binding activity to HLA-DQ2.5 when it is in the form of a complex with peptides which are irrelevant to gluten peptides.

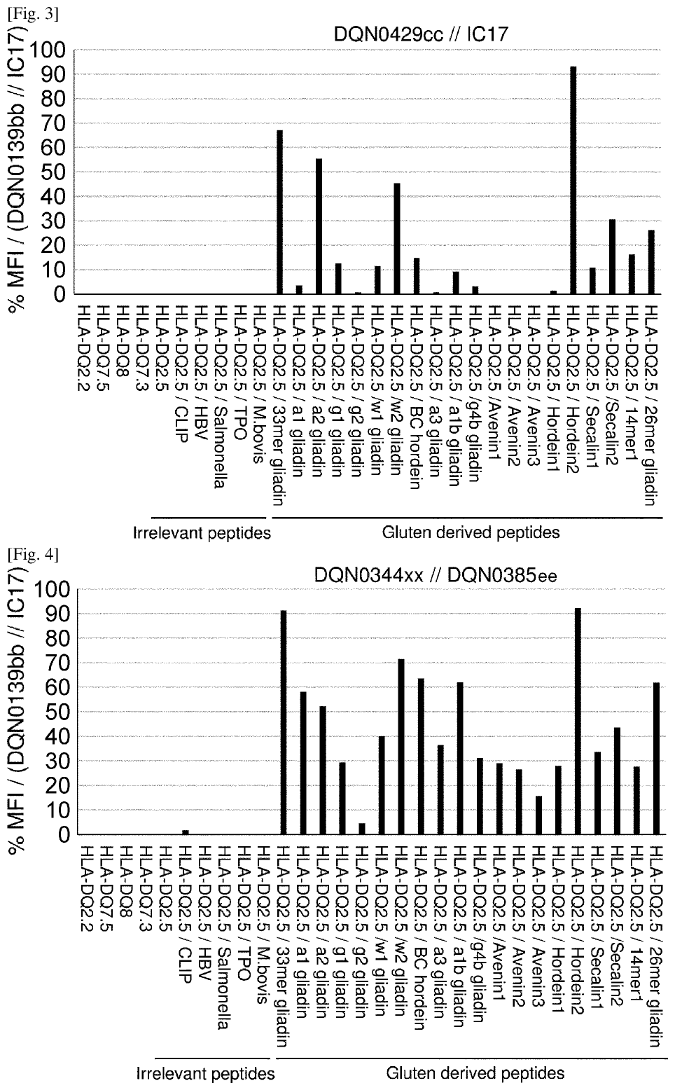

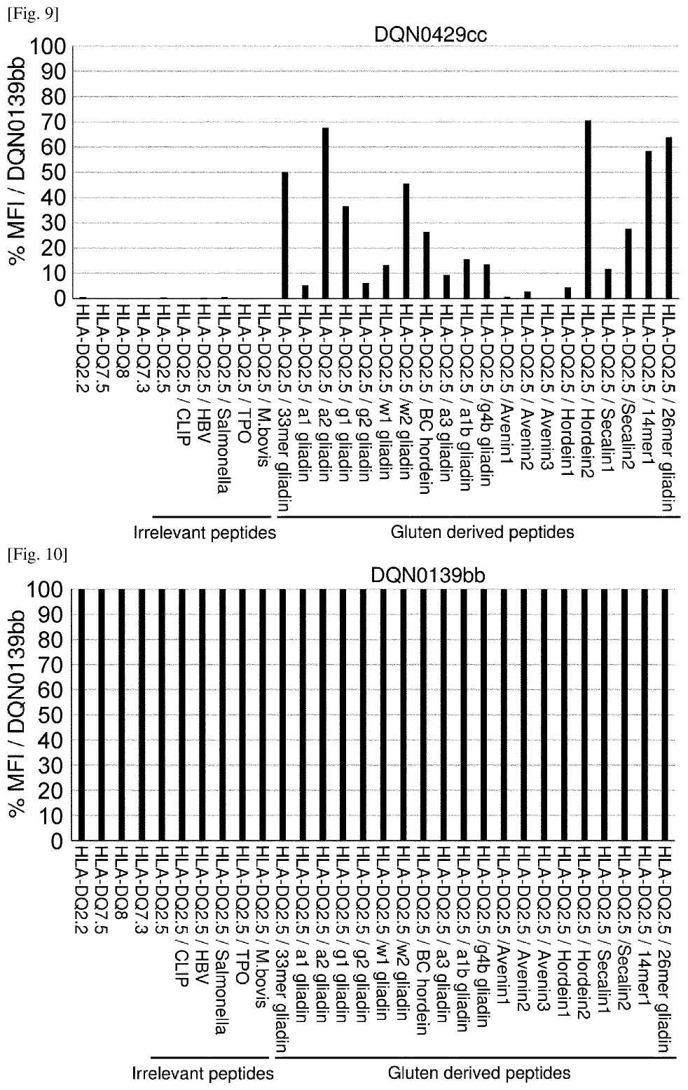

FIG. 3 and FIG. 9 show that DQN0429cc and DQN0429cc//IC17 have binding activity to HLA-DQ2.5 only when it is in the form of a complex with 33mer gliadin peptide, alpha 1 gliadin peptide, alpha 2 gliadin peptide, gamma 1 gliadin peptide, omega 1 gliadin peptide, omega 2 gliadin peptide, BC hordein peptide, alpha 1b gliadin peptide, gamma 4b gliadin peptide, hordein 1 peptide, hordein 2 peptide, secalin 1 peptide, secalin 2 peptide, 14mer1 peptide, and 26mer gliadin peptide. On the other hand, DQN0429cc and DQN0429cc//IC17 have substantially no binding activity to HLA-DQ2.5 when it is in the form of a complex with peptides which are irrelevant to gluten peptides.

FIG. 4 shows that DQN0344xx//DQN0385ee has binding activity to HLA-DQ2.5 only when it is in the form of a complex with 33mer gliadin peptide, alpha 1 gliadin peptide, alpha 2 gliadin peptide, gamma 1 gliadin peptide, gamma 2 gliadin peptide, omega 1 gliadin peptide, omega 2 gliadin peptide, BC hordein peptide, alpha 3 gliadin peptide, alpha 1b gliadin peptide, gamma 4b gliadin peptide, avenin 1 peptide, avenin 2 peptide, avenin 3 peptide, hordein 1 peptide, hordein 2 peptide, secalin 1 peptide, secalin 2 peptide, 14mer1 peptide, and 26mer gliadin peptide. On the other hand, DQN0344xx//DQN0385ee has substantially no binding activity to HLA-DQ2.5 when it is in the form of a complex with peptides which are irrelevant to gluten peptides.

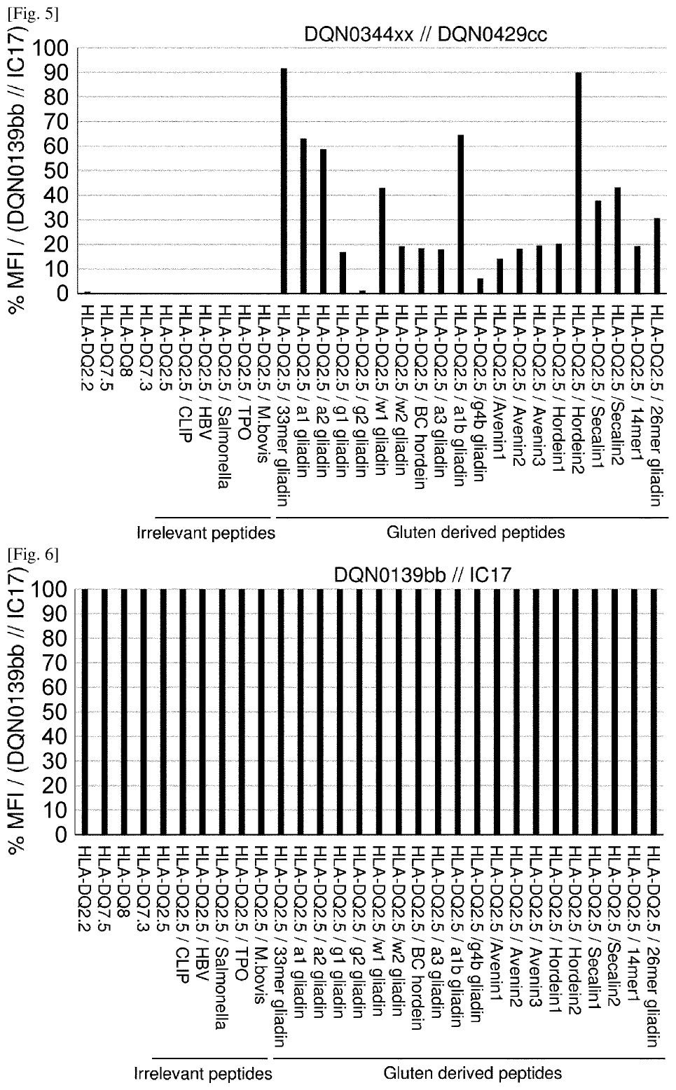

FIG. 5 shows that DQN0344xx//DQN0429cc has binding activity to HLA-DQ2.5 only when it is in the form of a complex with 33mer gliadin peptide, alpha 1 gliadin peptide, alpha 2 gliadin peptide, gamma 1 gliadin peptide, omega 1 gliadin peptide, omega 2 gliadin peptide, BC hordein peptide, alpha 3 gliadin peptide, alpha 1b gliadin peptide, gamma 4b gliadin peptide, avenin 1 peptide, avenin 2 peptide, avenin 3 peptide, hordein 1 peptide, hordein 2 peptide, secalin 1 peptide, secalin 2 peptide, 14mer1 peptide, and 26mer gliadin peptide. On the other hand, DQN0344xx//DQN0429cc has substantially no binding activity to HLA-DQ2.5 when it is in the form of a complex with peptides which are irrelevant to gluten peptides.

FIG. 6 and FIG. 10 shows that DQN0139bb and DQN0139bb//IC17 have binding activity to HLA-DQ2.5 in the form of a complex with or without any peptide.

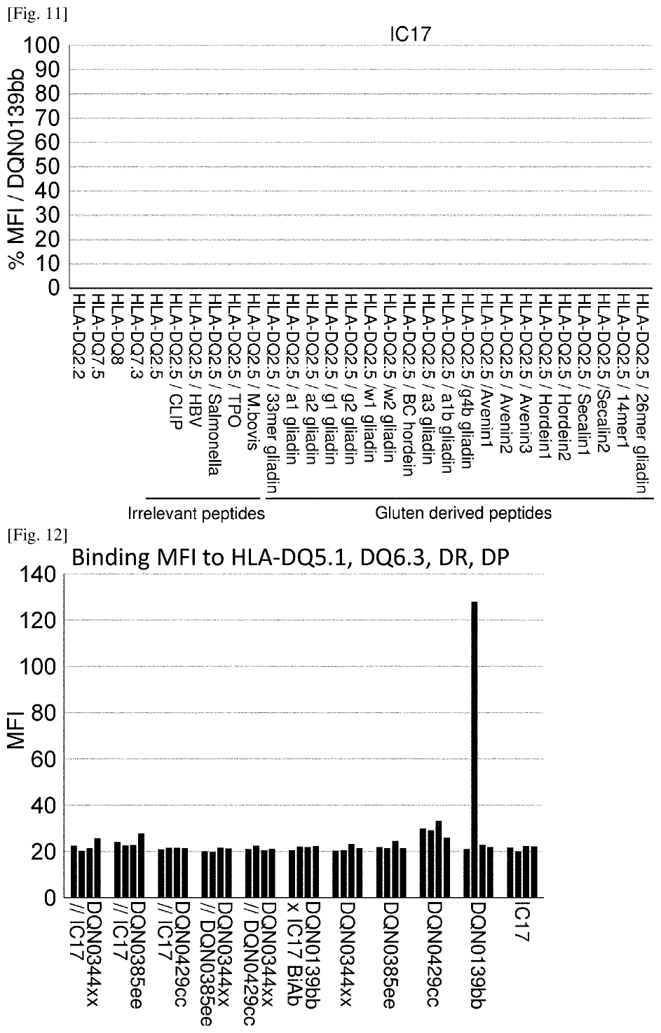

FIG. 11 shows analysis on binding of IC17 to complexes formed by HLA-DQ2.5 and gluten-derived peptides or irrelevant peptides. TC17 had substantially no binding activity to the tested complexes.

FIG. 12 shows analysis on binding of the antibodies to HLA molecules such as HLA-DQ5.1, HLA-DQ6.3, HLA-DR, and HLA-DP. The four bars, from left to right, show the results for HLA-DQ5.1, HLA-DQ6.3, HLA-DR, and HLA-DP, respectively. DQN0344xx//IC17, DQN0385ee//IC17, DQN0429cc//IC17, DQN0344xx//DQN0429cc, DQN0344xx, DQN0385ee, and DQN0429cc had substantially no binding activity to the tested HLA molecules.

### Example 6

Binding Analysis of the Antibodies to HLA-DQ2.5+ PBMC B Cell:

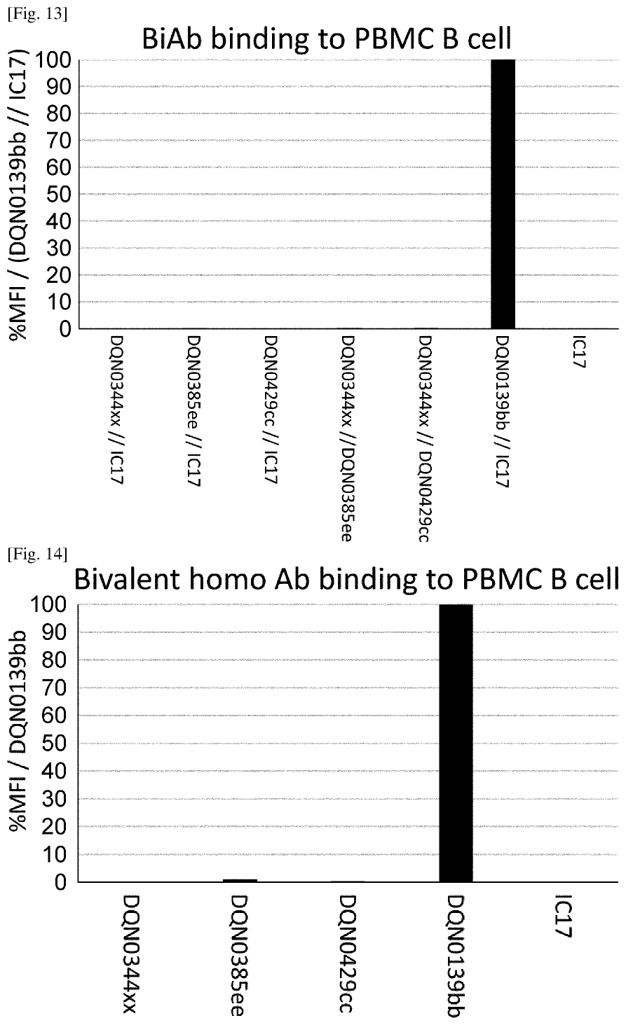

FIG. 13 and FIG. 14 show the binding of the anti-HLA-DQ antibodies to HLADQ2.5-positive PBMC B cell as determined by FACS. 20 microgram/mL of each of the anti-HLA-DQ antibodies was incubated with PBMC in the presence of human FcR blocking reagent (Miltenyi Biotech, Cat. 130-059-901) for 30 minutes at room temperature and washed with FACS buffer (2% FBS, 2 mM EDTA in PBS). Pacific Blue™ anti-human CD19 Antibody mouse IgG1k (Biolegend, Cat. 2043-09) and Alexa Fluor 555 labeled anti-human IgG Fc antibody (Reference Examples 1 to 3) was then added and incubated for 30 minutes at 4 degrees C., and this was washed with FACS buffer. Data acquisition was performed on LSRFortessa X-20 (Becton Dickinson), followed by analysis using the FlowJo software (Tree Star) and GraphPad Prism software (GraphPad). % MFI of bivalent antibodies was determined when taking a MFI value of IC17 as 0% and a MFI value of DQN0139bb/IC17 as 100%. % MFI of bivalent antibodies was determined when taking a MFI value of IC17 as 0% and a MFI value of DQN0139bb as 100%.

FIG. 13 shows that DQN0139bb/IC17 has binding activity to HLA-DQ2.5-positive PBMC B cell whereas DQN0344xx/IC17, DQN0385ee/IC17, DQN0429cc/IC17, DQN0344xx/DQN0385ee, and DQN0344xx/DQN0429cc have substantially no binding activity to the cell.

FIG. 14 shows that DQN0139bb has binding activity to HLA-DQ2.5-positive PBMC B cell whereas DQN0344xx, DQN0385ee, and DQN0429cc have substantially no binding activity to the cell.

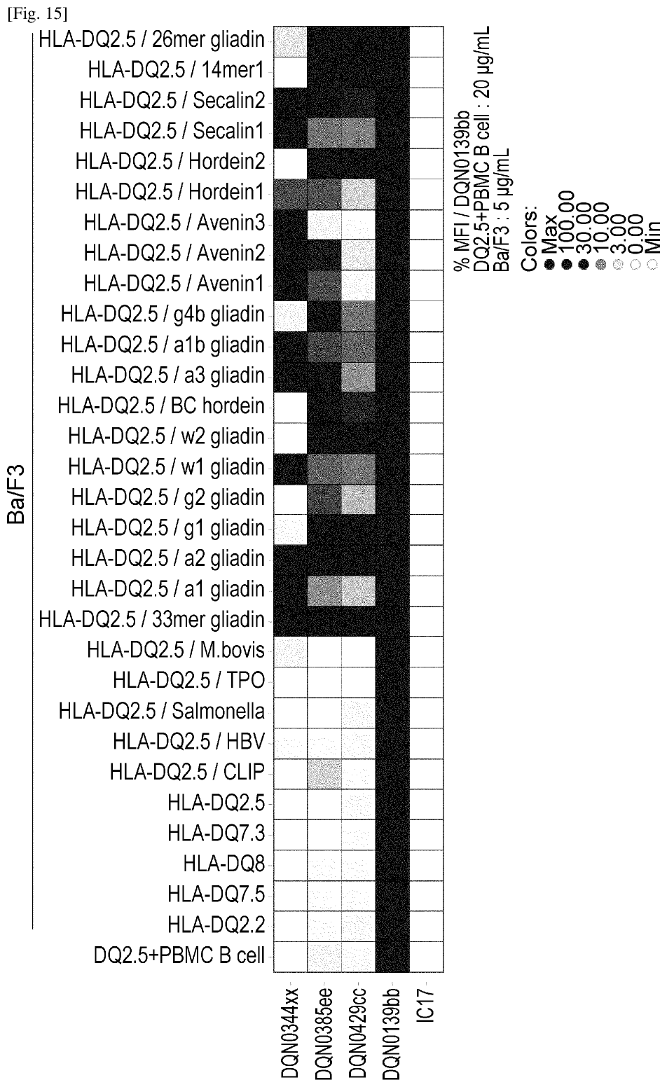

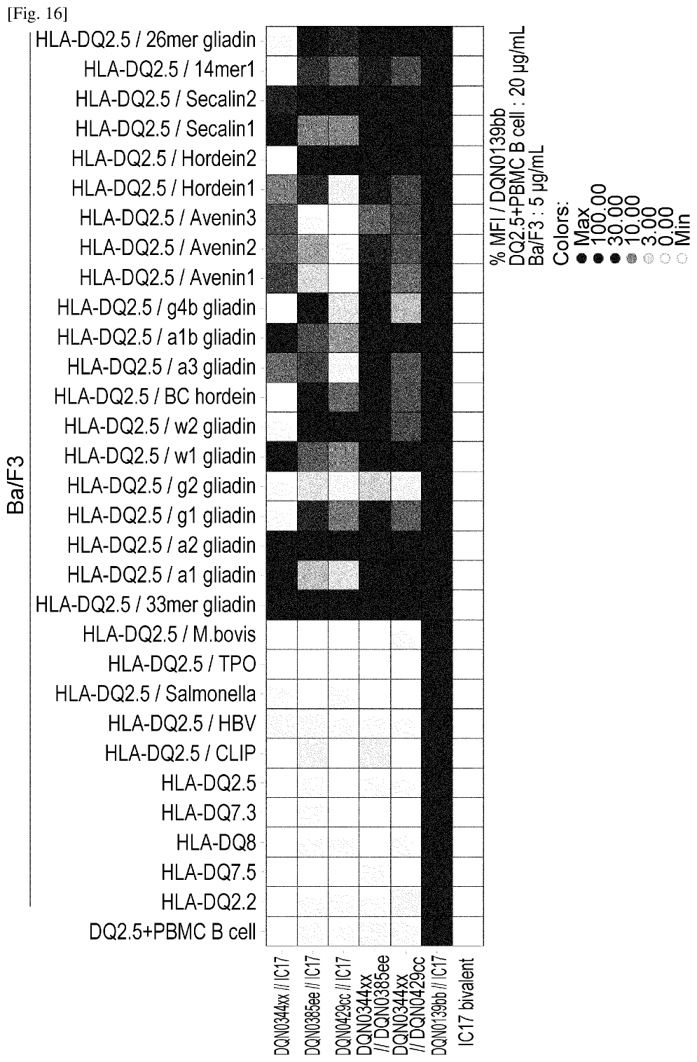

FIG. 15 and FIG. 16 is the summary of the above results. DQN0139bb and DQN0139bb//IC17 have binding activity to HLA-DQ2.5 in the form of a complex with or without any peptide, whereas DQN0344xx and DQN0344xx//IC17 have binding activity to HLA-DQ2.5 only when it is in the form of a complex with gluten-derived peptides, in particular 33mer gliadin peptide, alpha 1 gliadin peptide, alpha 2 gliadin peptide, omega 1 gliadin peptide, alpha 3 gliadin peptide, alpha 1b gliadin peptide, avenin 1 peptide, avenin 2 peptide, avenin 3 peptide, hordein 1 peptide, secalin 1 peptide, and secalin 2 peptide. On the other hand, DQN0344xx and DQN0344xx//IC17 have substantially no binding activity to HLA-DQ2.5 when it is in the form of a complex with peptides which are irrelevant to gluten peptides.

DQN0385ee and DQN0385ee//IC17 have binding activity to HLA-DQ2.5 only when it is in the form of a complex with 33mer gliadin peptide, alpha 1 gliadin peptide, alpha 2 gliadin peptide, gamma 1 gliadin peptide, gamma 2 gliadin peptide, omega 1 gliadin peptide, omega 2 gliadin peptide, BC hordein peptide, alpha 3 gliadin peptide, alpha 1b gliadin peptide, gamma 4b gliadin peptide, avenin 1 peptide, avenin 2 peptide, hordein 1 peptide, hordein 2 peptide, secalin 1 peptide, secalin 2 peptide, 14mer1 peptide, and 26mer gliadin peptide. On the other hand, DQN0385ee and DQN0385ee//IC17 have substantially no binding activity to HLA-DQ2.5 when it is in the form of a complex with peptides which are irrelevant to gluten peptides.

DQN0344xx//DQN0385ee has binding activity to HLA-DQ2.5 only when it is in the form of a complex with 33mer gliadin peptide, alpha 1 gliadin peptide, alpha 2 gliadin peptide, gamma 1 gliadin peptide, gamma 2 gliadin peptide, omega 1 gliadin peptide, omega 2 gliadin peptide, BC hordein peptide, alpha 3 gliadin peptide, alpha 1b gliadin peptide, gamma 4b gliadin peptide, avenin 1 peptide, avenin 2 peptide, avenin 3 peptide, hordein 1 peptide, hordein 2 peptide, secalin 1 peptide, secalin 2 peptide, 14mer1 peptide, and 26mer gliadin peptide. On the other hand, DQN0344xx//DQN0385ee has substantially no binding activity to HLA-DQ2.5 when it is in the form of a complex with peptides which are irrelevant to gluten peptides.

DQN0429cc and DQN0429cc//IC17 have binding activity to HLA-DQ2.5 only when it is in the form of a complex with 33mer gliadin peptide, alpha 1 gliadin peptide, alpha 2 gliadin peptide, gamma 1 gliadin peptide, omega 1 gliadin peptide, omega 2 gliadin peptide, BC hordein peptide, alpha 1b gliadin peptide, gamma 4b gliadin peptide, hordein 1 peptide, hordein 2 peptide, secalin 1 peptide, secalin 2 peptide, 14mer1 peptide, and 26mer gliadin peptide. On the other hand, DQN0429cc and DQN0429cc//IC17 have substantially no binding activity to HLA-DQ2.5 when it is in the form of a complex with peptides which are irrelevant to gluten peptides.

DQN0344xx//DQN0429cc has binding activity to HLA-DQ2.5 only when it is in the form of a complex with 33mer gliadin peptide, alpha 1 gliadin peptide, alpha 2 gliadin peptide, gamma 1 gliadin peptide, omega 1 gliadin peptide, omega 2 gliadin peptide, BC hordein peptide, alpha 3 gliadin peptide, alpha 1b gliadin peptide, gamma 4b gliadin peptide, avenin 1 peptide, avenin 2 peptide, avenin 3 peptide, hordein 1 peptide, hordein 2 peptide, secalin 1 peptide, secalin 2 peptide, 14mer1 peptide, and 26mer gliadin peptide. On the other hand, DQN0344xx//DQN0429cc has substantially no binding activity to HLA-DQ2.5 when it is in the form of a complex with peptides which are irrelevant to gluten peptides.

Numeral data for FIGS. 15 and 16 are shown in Tables 4 and 5, respectively.

TABLE 4

| | HLA- | | | | | | | | | | | HLA- |
|---|---|---|---|---|---|---|---|---|---|---|---|---|
| | DQ2.5 + | | | | | | HLA- | HLA- | HLA- | HLA- | HLA- | DQ2.5/ |
| | PBMC | HLA- | HLA- | HLA- | HLA- | HLA- | DQ2.5/ | DQ2.5/ | DQ2.5/ | DQ2.5/ | DQ2.5/ | 33mer |
| | B cell | DQ2.2 | DQ7.5 | DQ8 | DQ7.3 | DQ2.5 | CLIP | HBV | Salmonella | TPO | M. Bovis | gliadin |
| DQN0344xx | −0.1 | −0.1 | 0.0 | 0.0 | 0.0 | 0.0 | 0.1 | 0.1 | 0.0 | 0.1 | 1.1 | 107.1 |
| DQN0385ee | 1.0 | 0.2 | 0.2 | 0.1 | 0.1 | 0.1 | 4.4 | 0.2 | 0.1 | 0.0 | 0.0 | 56.2 |
| DQN0429cc | 0.4 | 0.7 | 0.2 | 0.2 | 0.2 | 0.4 | 0.2 | 0.4 | 0.6 | 0.0 | 0.0 | 50.2 |
| DQN0139bb | 100.0 | 100.0 | 100.0 | 100.0 | 100.0 | 100.0 | 100.0 | 100.0 | 100.0 | 100.0 | 100.0 | 100.0 |
| IC17 | 0.0 | 0.0 | 0.0 | 0.0 | 0.0 | 0.0 | 0.0 | 0.0 | 0.0 | 0.0 | 0.0 | 0.0 |

| | HLA- | | | | | | HLA- | | HLA- | HLA- |
|---|---|---|---|---|---|---|---|---|---|---|
| | DQ2.5/ | HLA- | HLA- | HLA- | HLA- | HLA- | DQ2.5/ | HLA- | DQ2.5/ | DQ2.5/ |
| | a1 | DQ2.5/ | DQ2.5/ | DQ2.5/ | DQ2.5/ | DQ2.5/ | BC | DQ2.5/ | a1b | g4b |
| | gliadin | a2 gliadin | g1 gliadin | g2 gliadin | w1 gliadin | w2 gliadin | hordein | a3 gliadin | gliadin | gliadin |
| DQN0344xx | 77.1 | 69.5 | 0.4 | 0.0 | 49.5 | 0.1 | 0.0 | 42.2 | 66.6 | 1.0 |
| DQN0385ee | 9.7 | 70.9 | 57.7 | 20.6 | 16.5 | 53.0 | 61.8 | 59.7 | 20.3 | 53.6 |
| DQN0429cc | 5.4 | 67.7 | 36.6 | 6.2 | 13.4 | 45.5 | 26.4 | 9.4 | 15.7 | 13.6 |
| DQN0139bb | 100.0 | 100.0 | 100.0 | 100.0 | 100.0 | 100.0 | 100.0 | 100.0 | 100.0 | 100.0 |
| IC17 | 0.0 | 0.0 | 0.0 | 0.0 | 0.0 | 0.0 | 0.0 | 0.0 | 0.0 | 0.0 |

| | HLA- | | HLA- | | HLA- | HLA- | | HLA- | |
|---|---|---|---|---|---|---|---|---|---|
| | DQ2.5/ | HLA-DQ2.5/ | DQ2.5/ | HLA-DQ2.5/ | DQ2.5/ | DQ2.5/ | HLA-DQ2.5/ | DQ2.5/ | HLA-DQ2.5/ |
| | Avenin1 | Avenin2 | Avenin3 | Hordein1 | Hordein2 | Secalinl | Secalin2 | 14mer1 | 26mer gliadin |
| DQN0344xx | 60.9 | 48.6 | 48.4 | 18.8 | 0.1 | 52.2 | 37.6 | 0.0 | 3.2 |
| DQN0385ee | 19.1 | 28.0 | 1.1 | 18.2 | 71.4 | 13.7 | 32.5 | 70.7 | 70.8 |
| DQN0429cc | 0.8 | 2.9 | 0.1 | 4.5 | 70.6 | 11.8 | 27.7 | 58.5 | 64.0 |
| DQN0139bb | 100.0 | 100.0 | 100.0 | 100.0 | 100.0 | 100.0 | 100.0 | 100.0 | 100.0 |
| IC17 | 0.0 | 0.0 | 0.0 | 0.0 | 0.0 | 0.0 | 0.0 | 0.0 | 0.0 |

TABLE 5

| | HLA- | | | | | | | | | | |
|---|---|---|---|---|---|---|---|---|---|---|---|
| | DQ2.5 + | | | | | | HLA- | HLA- | HLA- | HLA- | HLA- |
| | PBMC | HLA- | HLA- | HLA- | HLA- | HLA- | DQ2.5/ | DQ2.5/ | DQ2.5/ | DQ2.5/ | DQ2.5/ |
| | B cell | DQ2.2 | DQ7.5 | DQ8 | DQ7.3 | DQ2.5 | CLIP | HBV | Salmonella | TPO | M. Bovis |
| DQN0344xx//IC17 | 0.0 | 0.1 | 0.0 | 0.0 | 0.1 | 0.1 | 0.0 | 0.3 | 0.1 | 0.0 | 0.1 |
| DQN0385ee//IC17 | 0.3 | 0.3 | 0.1 | 0.1 | 0.1 | 0.1 | 1.3 | 0.5 | 0.0 | 0.0 | 0.0 |
| DQN0429cc//IC17 | 0.1 | 0.3 | 0.1 | 0.1 | 0.1 | 0.1 | 0.0 | 0.1 | 0.2 | 0.0 | 0.0 |
| DQN0344xx//DQN0385ee | 0.4 | 0.1 | 0.1 | 0.1 | 0.0 | 0.1 | 1.8 | 0.2 | 0.0 | 0.0 | 0.1 |
| DQN0344xx//DQN0429cc | 0.4 | 0.8 | 0.0 | 0.1 | 0.1 | 0.1 | 0.1 | 0.1 | 0.0 | 0.0 | 0.3 |
| DQN0139bb//IC17 | 100.0 | 100.0 | 100.0 | 100.0 | 100.0 | 100.0 | 100.0 | 100.0 | 100.0 | 100.0 | 100.0 |
| IC17 bivalent | 0.0 | 0.0 | 0.0 | 0.0 | 0.0 | 0.0 | 0.0 | 0.0 | 0.0 | 0.0 | 0.0 |

| | HLA- | HLA- | HLA- | HLA- | HLA- | HLA- | HLA- | HLA- | | HLA- | HLA- |
|---|---|---|---|---|---|---|---|---|---|---|---|
| | DQ2.5/ | DQ2.5/ | DQ2.5/ | DQ2.5/ | DQ2.5/ | DQ2.5/ | DQ2.5/ | DQ2.5/ | HLA- | DQ2.5/ | DQ2.5/ |
| | 33mer | a1 | a2 | g1 | g2 | w1 | w2 | BC | DQ2.5/ | a1b | g4b |
| | gliadin | gliadin | gliadin | gliadin | gliadin | gliadin | gliadin | hordein | a3 gliadin | gliadin | gliadin |
| DQN0344xx//lCl7 | 99.1 | 60.1 | 90.4 | 0.2 | 0.2 | 37.5 | 0.2 | 0.0 | 14.6 | 72.2 | 0.1 |
| DQN0385ee//IC17 | 73.9 | 6.0 | 85.6 | 26.0 | 3.4 | 17.2 | 75.2 | 68.1 | 20.9 | 19.2 | 32.4 |
| DQN0429cc//IC17 | 67.1 | 3.5 | 55.5 | 12.6 | 0.8 | 11.5 | 45.4 | 14.9 | 0.8 | 9.2 | 3.2 |
| DQN0344xx//DQN0385ee | 91.3 | 58.1 | 52.3 | 29.4 | 4.6 | 40.0 | 71.5 | 63.5 | 36.4 | 61.9 | 31.1 |
| DQN0344xx//DQN0429cc | 91.6 | 63.1 | 58.7 | 16.9 | 1.3 | 43.0 | 19.3 | 18.5 | 18.0 | 64.6 | 6.2 |
| DQN0139bb//IC17 | 100.0 | 100.0 | 100.0 | 100.0 | 100.0 | 100.0 | 100.0 | 100.0 | 100.0 | 100.0 | 100.0 |
| IC17 bivalent | 0.0 | 0.0 | 0.0 | 0.0 | 0.0 | 0.0 | 0.0 | 0.0 | 0.0 | 0.0 | 0.0 |

| | | | | | | | | | HLA- |
|---|---|---|---|---|---|---|---|---|---|
| | HLA- | HLA- | HLA- | HLA- | | HLA- | HLA- | HLA- | DQ2.5/ |
| | DQ2.5/ | DQ2.5/ | DQ2.5/ | DQ2.5/ | HLA-DQ2.5/ | DQ2.5/ | DQ2.5/ | DQ2.5/ | 26mer |
| | Avenin1 | Avenin2 | Avenin3 | Hordein1 | Hordein2 | Secalin1 | Secalin2 | 14mer1 | gliadin |
| DQN0344xx//lCl7 | 20.7 | 16.8 | 17.8 | 11.9 | 0.0 | 31.6 | 26.5 | 0.0 | 0.2 |
| DQN0385ee//IC17 | 4.0 | 8.3 | 0.2 | 24.7 | 98.3 | 11.8 | 34.4 | 23.3 | 70.5 |
| DQN0429cc//IC17 | 0.0 | 0.1 | 0.0 | 1.5 | 93.2 | 10.9 | 30.6 | 16.3 | 26.2 |
| DQN0344xx//DQN0385ee | 29.1 | 26.6 | 15.6 | 28.0 | 92.2 | 33.6 | 43.5 | 27.6 | 61.8 |
| DQN0344xx//DQN0429cc | 14.2 | 18.3 | 19.6 | 20.3 | 90.0 | 37.8 | 43.3 | 19.4 | 30.7 |
| DQN0139bb//IC17 | 100.0 | 100.0 | 100.0 | 100.0 | 100.0 | 100.0 | 100.0 | 100.0 | 100.0 |
| IC17 bivalent | 0.0 | 0.0 | 0.0 | 0.0 | 0.0 | 0.0 | 0.0 | 0.0 | 0.0 |

### Example 7

Cell-Based Neutralizing Assay:

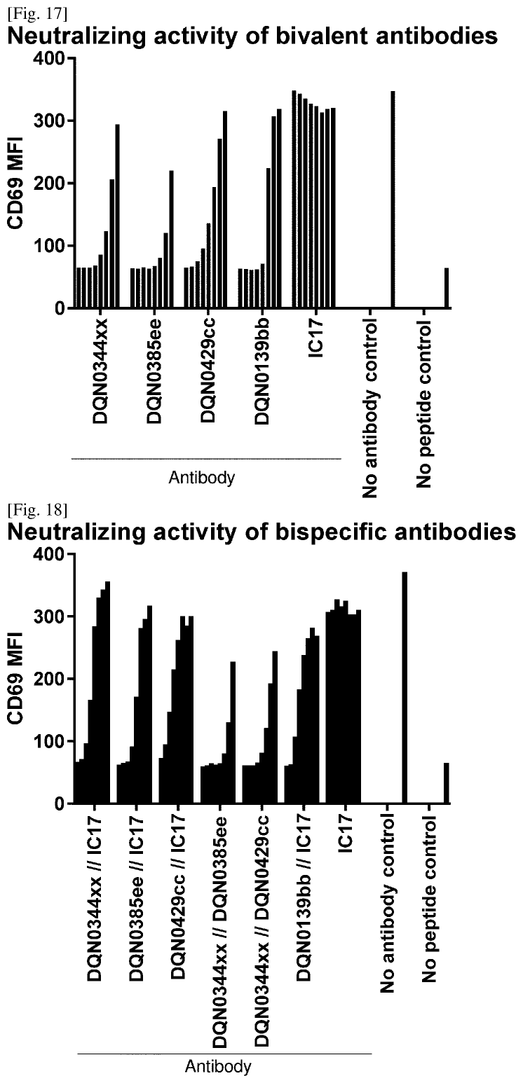

Cell-based neutralizing activity was confirmed. Epstein-Barr virus (EBV)-transformed lymphoblastoid cell line with HLA-DQ2.5 (ECACC, IHW9088) was distributed in 96 well plates (Corning, 3799). Chemically synthesized 33mer gliadin peptide (Genscript, LQLQPFPQPELPYPQPELPYPQPELPYPQPQPF (SEQ ID NO: 39)) and serially-diluted anti-HLA-DQ antibodies and D2 TCR-expressing J.RT3-T3.5 cells were then added and cultured at 37 degrees C., at 5% CO₂ for overnight. The final concentration of 33mer gliadin peptide was 200 microgram/mL, IHW9088 was 3.0×10⁴ cells/well, D2 TCR-expressing J.RT3-T3.5 cells was 1.0×10⁵ cells/well, and the final assay volume was 100 micro L/well. After overnight culture, cells were harvested and washed by FACS buffer (2% FBS, 2 mM EDTA in PBS). 40 fold-diluted APC anti-human CD20 antibody (Biolegend, 302310), and 40 fold-diluted Brilliant Violet 421 anti-human CD69 Antibody (Biolegend, 310930) were then added and incubated for 30 minutes at 4 degrees C., and washed and re-suspended with FACS buffer. Data acquisition was performed on a LSR Fortessa (Becton Dickinson), followed by analysis using FlowJo software (Tree Star) and GraphPad Prism software (GraphPad) to determine neutralizing activity of anti-HLA-DQ antibodies on the activation of D2 TCR-expressing J.RT3-T3.5 cells. CD69 expression on J.RT3-T3.5 cells was used for an activation marker. As shown in FIGS. 17 and 18, all tested anti-HLA DQ2.5 antibodies inhibited the activation of D2 TCR-expressing T cells induced by 33mer gliadin peptide.

### Example 8

The affinity of anti-HLA-DQ2.5 antibodies binding to human HLA-DQ2.5/33mer gliadin peptide complex, HLA-DQ2.5/gamma 2 gliadin peptide complex and HLADQ2.5/BC Hordein gliadin peptide complex at pH 7.4 was determined at 37 degrees C. using Biacore T200 instrument (GE Healthcare). Anti-human Fc (GE Healthcare) was immobilized onto all flow cells of a CM4 sensor chip using amine coupling kit (GE Healthcare). All antibodies and analytes were prepared in ACES (pH 7.4) containing 20 mM ACES, 150 mM NaCl, 0.05% Tween 20, 0.005% NaN₃. Each antibody was captured onto the sensor surface by anti-human Fc. Antibody capture levels were aimed at 200 resonance unit (RU). Recombinant human HLA-DQ2.5/33mer gliadin peptide complex and HLA-DQ2.5/gamma 2 gliadin peptide complex were injected at 50 to 800 nM prepared by two-fold serial dilution, followed by dissociation. Recombinant human HLA-DQ2.5/BC Hordein gliadin peptide complex was injected at 25 to 400 nM prepared by two-fold serial dilution, followed by dissociation. Sensor surface was regenerated each cycle with 3M MgCl₂. Binding affinity was determined by processing and fitting the data to 1:1 binding model using Biacore T200 Evaluation software (GE Healthcare).

The affinity of anti-HLA-DQ2.5 antibodies binding to human HLA-DQ2.5/33mer gliadin peptide complex, HLA-DQ2.5/gamma 2 gliadin peptide complex and HLADQ2.5/BC Hordein gliadin peptide complex is shown in Table 6.

TABLE 6

| | HLA-DQ2.5/33mer gliadin peptide | | | HLA-DQ2.5/γ2 gliadin peptide | | | HLA-DQ2.5/BC Hordein peptide | | |
|---|---|---|---|---|---|---|---|---|---|
| | complex | | | complex | | | complex | | |
| Ab name | ka (M⁻¹s⁻¹) | kd (s⁻¹) | KD (M) | ka (M⁻¹s⁻¹) | kd (s⁻¹) | KD (M) | ka (M⁻¹s⁻¹) | kd (s⁻¹) | KD (M) |
| DQN0344xx//IC17 | 1.70E+05 | 1.20E−03 | 7.04E−09 | N.D. | N.D. | N.D. | N.D. | N.D. | N.D. |
| DQN0385ee//IC17 | 5.73E+04 | 3.11E−04 | 5.43E−09 | 1.27E+05 | 1.28E−02 | 1.01E−07 | 1.98E+05 | 7.39E−04 | 3.73E−09 |
| DQN0429cc//IC17 | 3.73E+04 | 5.86E−04 | 1.57E−08 | 6.10E+04 | 2.66E−02 | 4.35E−07 | 9.64E+04 | 5.57E−03 | 5.78E−08 |
| DQN0344xx//DQN0385ee | 9.99E+04 | 7.03E−04 | 7.03E−09 | 1.26E+05 | 1.18E−02 | 9.38E−08 | 2.00E+05 | 7.36E−04 | 3.68E−09 |
| DQN0344xx//DQN0429cc | 8.01E+04 | 7.33E−04 | 9.16E−09 | 6.29E+04 | 2.33E−02 | 3.70E−07 | 9.88E+04 | 5.12E−03 | 5.18E−08 |
| DQN0139bb//IC17 | 6.85E+04 | 1.76E−04 | 2.56E−09 | 1.71E+05 | 2.91E−04 | 1.70E−09 | 1.77E+05 | 2.16E−04 | 1.22E−09 |
| DQN0344xx | 1.90E+05 | 1.10E−03 | 5.77E−09 | N.D. | N.D. | N.D. | N.D. | N.D. | N.D. |
| DQN0385ee | 6.32E+04 | 2.92E−04 | 4.63E−09 | 1.37E+05 | 1.10E−02 | 8.05E−08 | 2.12E+05 | 6.75E−04 | 3.19E−09 |
| DQN0429cc | 3.93E+04 | 5.40E−04 | 1.37E−08 | 5.92E+04 | 2.10E−02 | 3.55E−07 | 1.02E+05 | 5.28E−03 | 5.18E−08 |
| DQN0139bb | 7.36E+04 | 1.90E−04 | 2.58E−09 | 1.95E+05 | 2.60E−04 | 1.34E−09 | 1.94E+05 | 2.36E−04 | 1.21E−09 |

### Example 9

9.1 Establishment of TCR KO Jurkat NFAT-Luc Cell Line

Ribonucleoprotein (RNP) complex, which is composed of Cas9 and single guide RNAs targeting TCR constant region (Blood. 2018; 131:311-22) was introduced to NFAT-RE-luc2 Jurkat cell line (Promega corporation, CS176401) by electroporation (LONZA, Nucleofector 2b). All single guide RNAs for TCR alpha chain and TCR beta chain were mixed and introduced simultaneously. RNP introduced cells were cultured in media containing Hygromycin B, followed by single cell cloning with FACS Aria III (Becton, Dickinson and Company). TCR alpha chain and TCR beta chain sequences were then checked and identified Jurkat NFAT-Luc derived clones which TCR alpha chain and TCR beta chain were knocked out. Established clone was named TCR KO Jurkat NFAT-Luc.

9.2 Establishment of TCR KO Jurkat NFAT-Luc Cell Line Expressing DQ2.5/Gluten Peptide Restricted TCR

Amino acid sequence information of DQ2.5/alpha 1 gliadin restricted TCR (TCC ID: 387.9), DQ2.5/alpha 1b gliadin restricted TCR (TCC ID: 370.2.25), DQ2.5/omega 1 gliadin restricted TCR (TCC ID: 442P.C.21), DQ2.5/omega 2 gliadin restricted TCR (TCC ID: 578.42), DQ2.5/gamma 1 gliadin restricted TCR (TCC ID: 820.27), DQ2.5/gamma 2 gliadin restricted TCR (TCC ID: 430.1.41), DQ2.5/gamma 4a gliadin restricted TCR (TCC ID: 430.1.36) was obtained from Oslo University based on material transfer agreement. Amino acid sequence information of DQ2.5/alpha 2 gliadin restricted TCR (D2 TCR) was obtained from Nat Struct Mol Biol. 2014; 21:480-8, and amino acid sequence information of DQ2.5/BC hordein restricted TCR (TCC ID: 1468.2) was obtained from Eur J Immunol. 2020; 50: 256-269.

Each TCR beta chain sequence was linked with corresponding TCR alpha chain sequence by 2A self-cleaving peptide sequence (P2A, amino acid sequence: GSGAT-NFSLLKQAGDVEENPGP, SEQ ID NO: 93). All TCR alpha chain and TCR beta chain have these own native signal peptide sequence except for DQ2.5/gamma 2 gliadin restricted TCR and DQ2.5/alpha 2 gliadin restricted TCR. Native signal sequence of DQ2.5/gamma 2 gliadin restricted TCR was replaced by Campath signal sequence (MGWSCIILFLVATATGVHS, SEQ ID NO: 37). Campath signal sequence (MGWSCIILFLVATATGVHS, SEQ ID NO: 37) was also attached N-terminus of DQ2.5/alpha 2 gliadin restricted TCR alpha chain beta chain. Each codon optimized TCR beta chain-P2A-TCR alpha chain cDNA was inserted into the expression vector pCXZD1 (US/20090324589). For DQ2.5/alpha 1 gliadin restricted TCR, DQ2.5/alpha 2 gliadin restricted TCR (D2 TCR), DQ2.5/omega 1 gliadin restricted TCR, DQ2.5/omega 2 gliadin restricted TCR, DQ2.5/gamma 1 gliadin restricted TCR, DQ2.5/gamma 2 gliadin restricted TCR, DQ2.5/BC hordein restricted TCR, each TCR beta chain-P2A-TCR alpha chain-pCXZD1 was introduced into TCR KO Jurkat NFAT-Luc by electroporation (LONZA, 4D-Nucleofector). Transfected cells were then cultured in media containing Zeocin and Hygromycin B, followed by single cell cloning of TCR positive fraction (determined by staining with Anti-TCRalphabeta antibody, Miltenyi Biotech) with FACS Aria III (Becton, Dickinson and Company). Established clones were named alpha 1 glaidin TCR Jurkat NFAT-Luc when DQ2.5/alpha 1 gliadin restricted TCR was introduced, omega 1 glaidin TCR Jurkat NFAT-Luc when DQ2.5/omega 1 gliadin restricted TCR was introduced, omega 2 glaidin TCR Jurkat NFAT-Luc when DQ2.5/omega 2 gliadin restricted TCR was introduced, gamma 1 glaidin TCR Jurkat NFAT-Luc when DQ2.5/gamma 1 gliadin restricted TCR was introduced, gamma 2 glaidin TCR Jurkat NFAT-Luc when DQ2.5/gamma 2 gliadin restricted TCR was introduced, D2 TCR Jurkat NFAT-Luc when DQ2.5/alpha 2 gliadin restricted TCR (D2) was introduced, and BC hordein TCR Jurkat NFAT-Luc when DQ2.5/BC hordein restricted TCR was introduced. For DQ2.5/alpha 1b gliadin restricted TCR, DQ2.5/gamma 4a gliadin restricted TCR, each TCR beta chain-P2A-TCR alpha chain-pCXZD1 was introduced into TCR KO Jurkat NFAT-Luc by electroporation (LONZA, 4D-Nucleofector). Transfected cells were then cultured in media containing Zeocin and Hygromycin B and used directly as a transiently TCR expressed cell line. These transient TCR expressed cell lines were named alpha 1b glaidin TCR Jurkat NFAT-Luc when DQ2.5/alpha 1b gliadin restricted TCR was introduced, and gamma 4a glaidin TCR Jurkat NFAT-Luc when DQ2.5/gamma 4a gliadin restricted TCR was introduced.

### Example 10

Preparation of Tissue Transglutaminase Treated Pepsin Trypsin Digested Gliadin (tTG-PT Gliadin)

10 gram of gliadin (Sigma, G3375) was suspended by 100 mL of 0.2 N HCl, and followed by adjusting pH to pH7.4 by 2M NaOH. Then 201 milligram of pepsin (Sigma, P7012) was added, and stirred for 2 hours in water bath set to 37 degrees C. Pepsin treated gliadin was then treated by 201 milligram of trypsin (Sigma, T0303) and stirred for 4 hours in water bath set to 37 degrees C. To inactivate pepsin and trypsin, pepsin trypsin digested gliadin was incubated for 30 minutes at 98 degrees C. and then freeze-dried at −75 degrees C.

Pepsin trypsin digested gliadin was reconstituted by PBS to 1 mg/mL. Tissue transglutaminase (Sigma, T5398) was reconstituted by 1 mM CaCl₂)-PBS to 1 mg/mL. 1 mg/mL Pepsin trypsin digested gliadin was mixed with 1 mg/mL tissue transglutaminase at a ratio of 9 to 1 followed by incubating for 2 hours at 37 degrees C. to make 0.9 mg/mL of tTG-PT gliadin.

### Example 11

11.1

Inhibitory Effect of Anti-HLA DQ Antibodies on DQ2.5/Alpha 1 Gliadin Peptide Dependent Jurkat T Cell Activation was Confirmed. Epstein-Barr Virus (EBV)-Transformed Lymphoblastoid Cell Line Expressing HLA-DQ2.5 (ECACC, IHW9023) was Used as an Antigen Presenting Cell.

Mixture of IHW9023 cell and tTG-PT gliadin was distributed in 96 well plates (Corning, 3799). Serially-diluted anti-HLA-DQ antibodies and alpha 1 glaidin TCR Jurkat NFAT-Luc were then added and cultured at 37 degrees C., at 5% CO₂ for overnight. The final concentration of tTG2-PT gliadin was 100 microgram/mL, IHW9023 was 8.0×10⁴ cells/well, alpha 1 glaidin TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/alpha 1 gliadin peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

As shown in FIG. 22 and Table 7, DQN0344xx, DQN0139bb, DQN0344xx//DQN0385ee, DQN0344xx//DQN0429cc inhibited DQ2.5/alpha 1 gliadin peptide dependent Jurkat T cell activation by dose dependent manner. DQN0385xx also moderately inhibited DQ2.5/alpha 1 gliadin peptide dependent Jurkat T cell activation by dose dependent manner while IC50 value was not determined. On the other hand, DQN0429cc did not inhibit DQ2.5/alpha 1 gliadin peptide dependent Jurkat T cell activation even at the highest antibody concentration, 1000 ng/mL.

11.2

Inhibitory Effect of Anti-HLA DQ Antibodies on DQ2.5/Alpha 2 Gliadin Peptide Dependent Jurkat T Cell Activation was Confirmed. IHW9023 Cell was Used as an Antigen Presenting Cell.

Mixture of IHW9023 cell and tTG-PT gliadin was distributed in 96 well plates (Corning, 3799). Serially-diluted anti-HLA-DQ antibodies and D2 TCR Jurkat NFAT-Luc were then added and cultured at 37 degrees C., at 5% CO₂ for overnight. The final concentration of tTG2-PT gliadin was 50 microgram/mL, IHW9023 was 8.0×10⁴ cells/well, D2 TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/alpha 2 gliadin peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

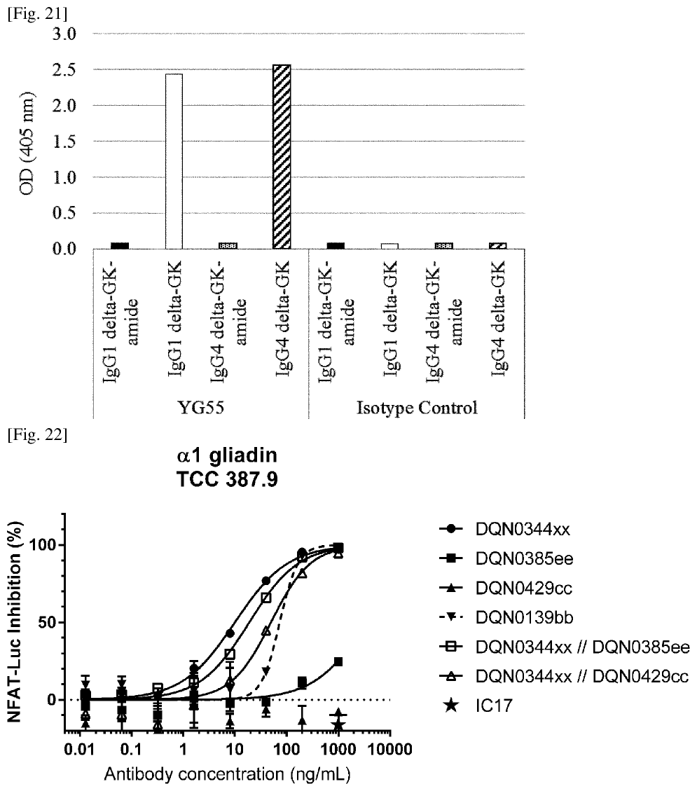

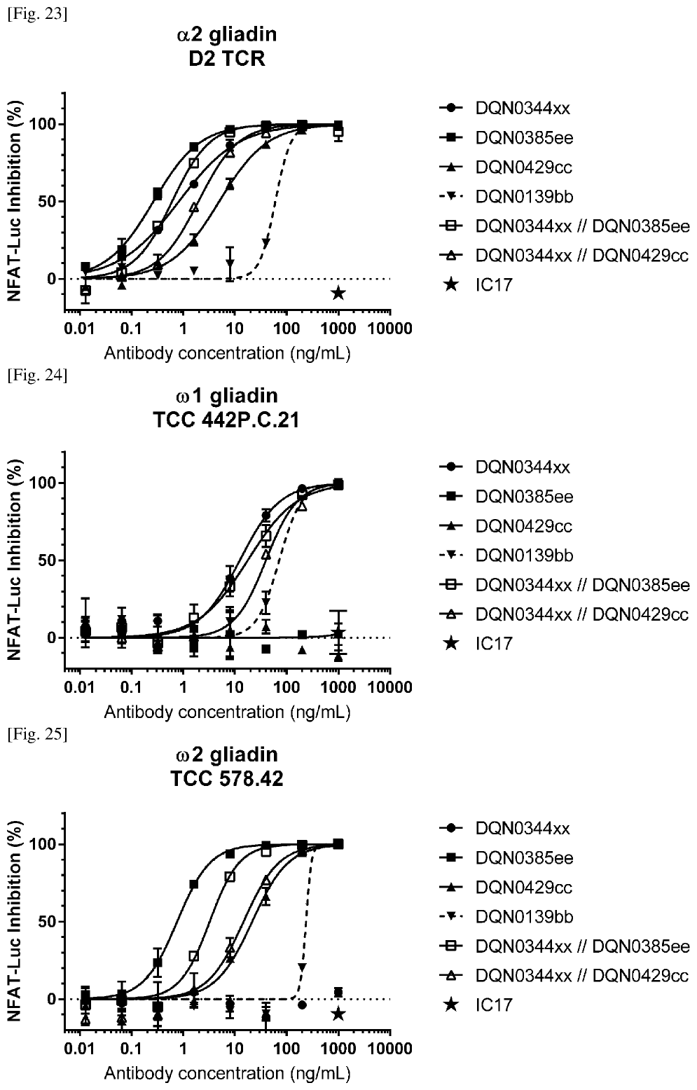

As shown in FIG. 23 and Table 7, All tested anti-HLA DQ antibodies inhibited DQ2.5/alpha 2 gliadin peptide dependent Jurkat T cell activation by dose dependent manner.

11.3

Inhibitory Effect of Anti-HLA DQ Antibodies on DQ2.5/Omega 1 Gliadin Peptide Dependent Jurkat T Cell Activation was Confirmed. IHW9023 Cell was Used as an Antigen Presenting Cell.

Mixture of IHW9023 cell and chemically synthesized omega gliadin W03E7 peptide (Genscript, EQPFPQPEQPFPWQP, SEQ ID NO: 94) was distributed in 96 well plates (Corning, 3799). Serially-diluted anti-HLA-DQ antibodies and omega 1 glaidin TCR Jurkat NFAT-Luc were then added and cultured at 37 degrees C., at 5% CO₂ for overnight. The final concentration of omega gliadin W03E7 peptide was 10 micro molar, IHW9023 was 8.0×10⁴ cells/well, omega 1 gliadin TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/omega 1 gliadin peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

As shown in FIG. 24 and Table 7, DQN0344xx, DQN0139bb, DQN0344xx//DQN0385ee, DQN0344xx//DQN0429cc inhibited DQ2.5/omega 1 gliadin peptide dependent Jurkat T cell activation by dose dependent manner. On the other hand, DQN0385xx and DQN0429cc did not inhibit DQ2.5/omega 1 gliadin peptide dependent Jurkat T cell activation even at the highest antibody concentration, 1000 ng/mL.

11.4

Inhibitory Effect of Anti-HLA DQ Antibodies on DQ2.5/Omega 2 Gliadin Peptide Dependent Jurkat T Cell Activation was Confirmed. IHW9023 Cell was Used as an Antigen Presenting Cell.

Mixture of IHW9023 cell and chemically synthesized omega gliadin W03E7 peptide (Genscript, EQPFPQPEQPFPWQP, SEQ ID NO: 94) was distributed in 96 well plates (Corning, 3799). Serially-diluted anti-HLA-DQ antibodies and omega 2 gliadin TCR Jurkat NFAT-Luc were then added and cultured at 37 degrees C., at 5% CO₂ for overnight. The final concentration of omega gliadin W03E7 peptide was 0.3 micro molar, IHW9023 was 8.0×10⁴ cells/well, omega 2 glaidin TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/omega 2 gliadin peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

As shown in FIG. 25 and Table 7, DQN0385ee, DQN0429cc, DQN0139bb, DQN0344xx//DQN0385ee, DQN0344xx//DQN0429cc inhibited DQ2.5/omega 2 gliadin peptide dependent Jurkat T cell activation by dose dependent manner. On the other hand, DQN0344xx did not inhibit DQ2.5/omega 2 gliadin peptide dependent Jurkat T cell activation even at the highest antibody concentration, 1000 ng/mL.

11.5

Inhibitory Effect of Anti-HLA DQ Antibodies on DQ2.5/Gamma 1 Gliadin Peptide Dependent Jurkat T Cell Activation was Confirmed. IHW9023 Cell was Used as an Antigen Presenting Cell.

Mixture of IHW9023 cell and tTG2-PT gliadin was distributed in 96 well plates (Corning, 3799). Serially-diluted anti-HLA-DQ antibodies and gamma 1 glaidin TCR Jurkat NFAT-Luc were then added and cultured at 37 degrees C., at 5% CO₂ for overnight. The final concentration of tTG2-PT gliadin was 50 microgram/mL, IHW9023 was 8.0×10⁴ cells/well, gamma 1 glaidin TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/gamma 1 gliadin peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

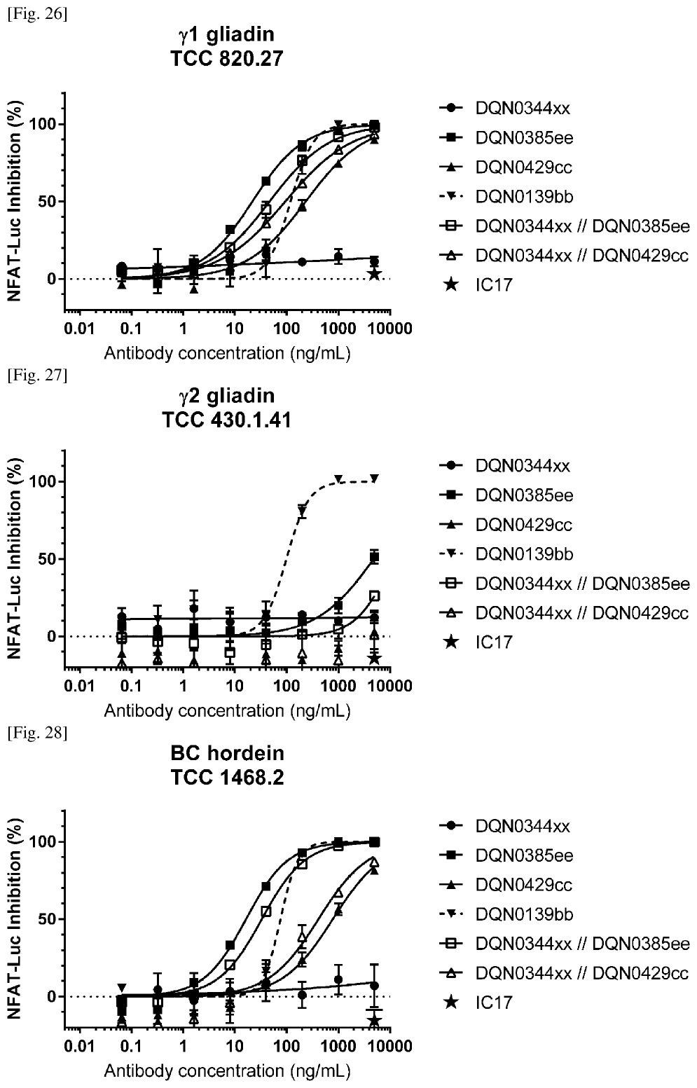

As shown in FIG. 26 and Table 7, DQN0385ee, DQN0429cc, DQN0139bb, DQN0344xx//DQN0385ee, DQN0344xx//DQN0429cc inhibited DQ2.5/gamma 1 gliadin peptide dependent Jurkat T cell activation by dose dependent manner. On the other hand, DQN0344xx did not inhibit DQ2.5/gamma 1 gliadin peptide dependent Jurkat T cell activation even at the highest antibody concentration, 1000 ng/mL.

11.6

Inhibitory Effect of Anti-HLA DQ Antibodies on DQ2.5/Gamma 2 Gliadin Peptide Dependent Jurkat T Cell Activation was Confirmed. IHW9023 Cell was Used as an Antigen Presenting Cell.

Mixture of IHW9023 cell and tTG2-PT gliadin was distributed in 96 well plates (Corning, 3799). Serially-diluted anti-HLA-DQ antibodies and gamma 2 glaidin TCR Jurkat NFAT-Luc were then added and cultured at 37 degrees C., at 5% CO₂ for overnight. The final concentration of tTG2-PT gliadin was 30 microgram/mL, IHW9023 was 8.0×10⁴ cells/well, gamma 2 glaidin TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/gamma 2 gliadin peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

As shown in FIG. 27 and Table 7, DQN0385ee and DQN0139bb inhibited DQ2.5/gamma 2 gliadin peptide dependent Jurkat T cell activation by dose dependent manner. DQN0344xx//DQN0385xx also moderately inhibited DQ2.5/gamma 2 gliadin peptide dependent Jurkat T cell activation by dose dependent manner while IC50 value was not determined. On the other hand, DQN0344xx, DQN0429cc, DQN0344xx//DQN0429cc did not inhibit DQ2.5/gamma 2 gliadin peptide dependent Jurkat T cell activation even at the highest antibody concentration, 5000 ng/mL.

11.7

Inhibitory Effect of Anti-HLA DQ Antibodies on DQ2.5/BC Hordein Peptide Dependent Jurkat T Cell Activation was Confirmed. IHW9023 Cell was Used as an Antigen Presenting Cell.

Mixture of IHW9023 cell and chemically synthesized BC hordein B08E2E7 peptide (Genscript, EPEQPIPEQPQPYPQQ, SEQ ID NO: 95) was distributed in 96 well plates (Corning, 3799). Serially-diluted anti-HLA-DQ antibodies and BC hordein TCR Jurkat NFAT-Luc were then added and cultured at 37 degrees C., at 5% CO₂ for overnight. The final concentration of B08E2E7 peptide was 0.2 micro molar, IHW9023 was 8.0×10⁴ cells/well, BC hordein TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/BC hordein peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

As shown in FIG. 28 and Table 7, DQN0385ee, DQN0429cc, DQN0139bb, DQN0344xx//DQN0385ee, and DQN0344xx//DQN0429cc inhibited DQ2.5/BC hordein peptide dependent Jurkat T cell activation by dose dependent manner. On the other hand, DQN0344xx did not inhibit DQ2.5/BC hordein peptide dependent Jurkat T cell activation even at the highest antibody concentration, 5000 ng/mL.

11.8

Inhibitory Effect of Anti-HLA DQ Antibodies on DQ2.5/Alpha 1b Peptide Dependent Jurkat T Cell Activation was Confirmed. IHW9023 Cell was Used as an Antigen Presenting Cell.

Mixture of IHW9023 cell and chemically synthesized 33mer gliadin peptide (Genscript, LQLQPFPQPELPYPQPELPYPQPELPYPQPQPF (SEQ ID NO: 39) was distributed in 96 well plates (Corning, 3799). Serially-diluted anti-HLA-DQ antibodies and alpha 1b glaidin TCR Jurkat NFAT-Luc were then added and cultured at 37 degrees C., at 5% CO₂ for overnight. The final concentration of 33mer gliadin peptide was 0.4 micro molar, IHW9023 was 8.0×10⁴ cells/well, alpha 1b glaidin TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/alpha 1b gliadin peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

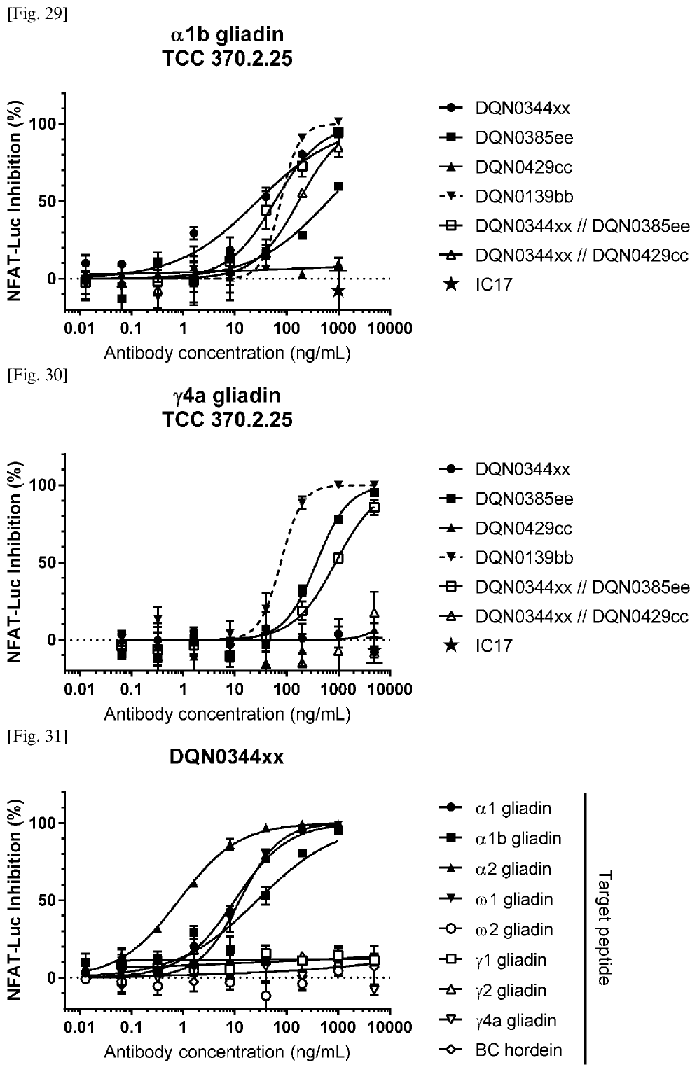

As shown in FIG. 29 and Table 7, DQN0344xx, DQN0385ee, DQN0139bb, DQN0344xx//DQN0385ee, and DQN0344xx//DQN0429cc inhibited DQ2.5/alpha 1b gliadin peptide dependent Jurkat T cell activation by dose dependent manner. On the other hand, DQN0429cc did not inhibit DQ2.5/BC hordein peptide dependent Jurkat T cell activation even at the highest antibody concentration, 5000 ng/mL.

11.9

Inhibitory Effect of Anti-HLA DQ Antibodies on DQ2.5/Gamma 4a Peptide Dependent Jurkat T Cell Activation was Confirmed. IHW9023 Cell was Used as an Antigen Presenting Cell.

Mixture of IHW9023 cell and chemically synthesized gamma 4a gliadin peptide (Genscript, FSQPEQEFPQPQ (SEQ ID NO: 96) was distributed in 96 well plates (Corning, 3799). Serially-diluted anti-HLA-DQ antibodies and gamma 4a glaidin TCR Jurkat NFAT-Luc were then added and cultured at 37 degrees C., at 5% CO₂ for overnight. The final concentration of gamma 4a gliadin peptide was 6 micro molar, IHW9023 was 8.0×10⁴ cells/well, gamma 4a glaidin TCR Jurkat NFAT-Luc was 2.0×10⁴ cells/well, and the final assay volume was 100 micro L/well. After overnight culture, 50 micro L of cultured cells were harvested and redistributed in OptiPlate-96 (PerkinElmer, 6005299). 50 micro L of Bio-Glo (Promega, G7491) was then added and incubated at room temperature for 10 minutes, and luminescence was measured with Envision (PerkinElmer), followed by analysis using Outlook Excel 2013 (Microsoft) and GraphPad Prism software (GraphPad) to determine the inhibitory effect of anti-HLA DQ antibodies on DQ2.5/gamma 4a gliadin peptide dependent Jurkat T cell activation. % inhibition of anti-HLA DQ antibodies was determined when taking a counts per second (CPS) of well in the absence of antigen without antibody as 100%, and a CPS of well in the presence of antigen without antibody as 0%. IC50 value was determined using XLfit Excel add-in software (IDBS).

As shown in FIG. 30 and Table 7, DQN0385ee, DQN0139bb, and DQN0344xx//DQN0385ee inhibited DQ2.5/gamma 4a gliadin peptide dependent Jurkat T cell activation by dose dependent manner. On the other hand, DQN0344xx, DQN0429cc and DQN0344xx//DQN0429cc did not inhibit DQ2.5/gamma 4a glaidin peptide dependent Jurkat T cell activation even at the highest antibody concentration, 5000 ng/mL.

As shown in FIG. 31 and Table 7, DQN0344xx inhibited DQ2.5/alpha 1 gliadin, alpha 2 glaidin, omega 1 gliadin, and alpha 1b gliadin peptide dependent Jurkat T cell activation by dose dependent manner.

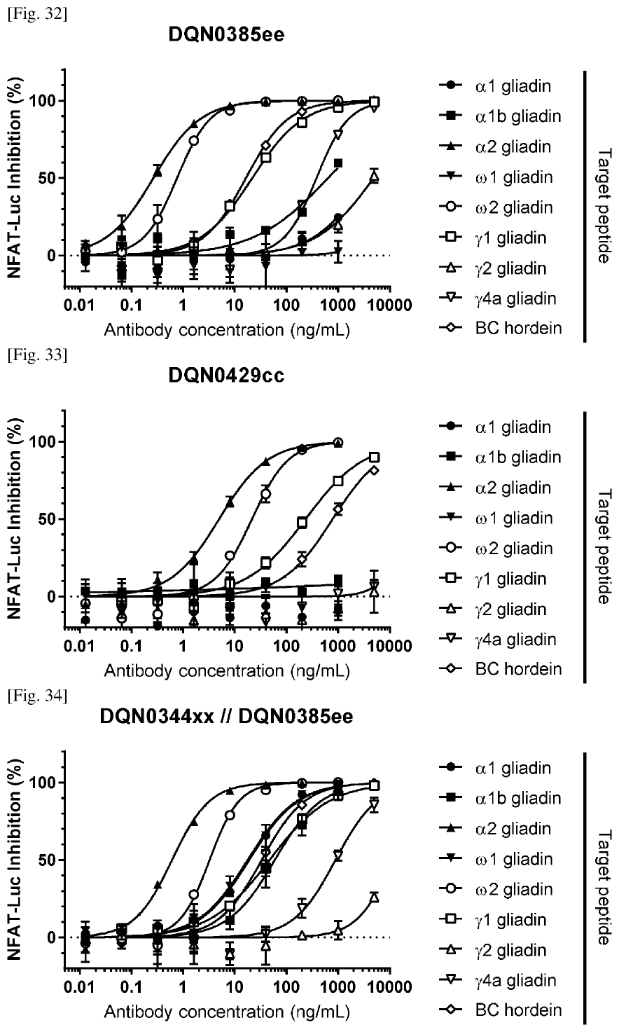

As shown in FIG. 32 and Table 7, DQN0385ee inhibited DQ2.5/alpha 2 glaidin, omega 2 gliadin, gamma 1 gliadin, gamma 2 gliadin, BC hordein, alpha 1b glaidin, and gamma 4a gliadin peptide dependent Jurkat T cell activation by dose dependent manner.

As shown in FIG. 33 and Table 7, DQN0429cc inhibited DQ2.5/alpha 2 glaidin, omega 2 gliadin, gamma 1 gliadin, and BC hordein peptide dependent Jurkat T cell activation by dose dependent manner.

As shown in FIG. 34 and Table 7, DQN0344xx//DQN0385ee inhibited DQ2.5/alpha 1 gliadin, alpha 2 glaidin, omega 1 gliadin, omega 2 gliadin, gamma 1 gliadin, BC hordein, alpha 1b glaidin, and gamma 4a gliadin peptide dependent Jurkat T cell activation by dose dependent manner. DQN0344xx//DQN0385ee moderately inhibited DQ2.5/gamma 2 gliadin peptide dependent Jurkat T cell activation by dose dependent manner while IC50 value was not determined.

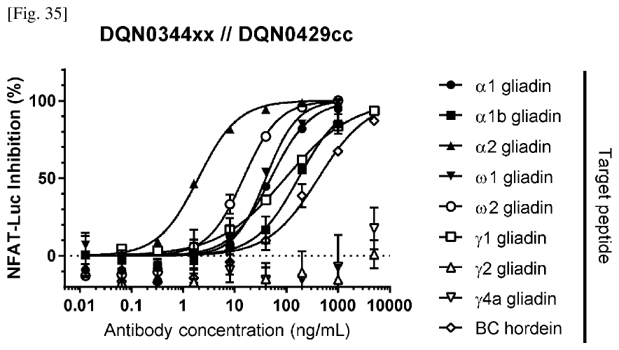

As shown in FIG. 35 and Table 7, DQN0344xx//DQN0429cc inhibited DQ2.5/alpha 1 gliadin, alpha 2 glaidin, omega 1 gliadin, omega 2 gliadin, gamma 1 gliadin, BC hordein, and alpha 1b glaidin peptide dependent Jurkat T cell activation by dose dependent manner.

IC50 value of anti HLA-DQ2.5 antibodies on DQ2.5/gluten peptides dependent Jurkat T cell activation is shown in Table 7.

TABLE 7

| | IC50 (ng/mL) | | | | | | | | | TCR |
|---|---|---|---|---|---|---|---|---|---|---|
| | α1 gliadin | α2 gliadin | ω1 gliadin | ω2 gliadin | γ1 gliadin | γ2 gliadin | BC hordein | α1b gliadin | γ4a gliadin | reactivity |
| | tTG2 PT | tTG2 PT | W03E7 | W03E7 | tTG2 PT | tTG2 PT | B08E2E7 | tTG2 PT | γ4a | Antigen |
| Ab name | gliadin | gliadin | peptide | peptide | gliadin | gliadin | peptide | gliadin | peptide | used |
| DQN0344xx | 9.98 | 0.820 | 12.4 | N.D. | N.D. | N.D. | N.D. | 27.2 | N.D. | |
| DQN0385ee | N.D. | 0.264 | N.D. | 0.758 | 21.7 | 4900 | 16.4 | 640 | 381 | |
| DQN0429cc | N.D. | 4.97 | N.D. | 21.5 | 235 | N.D. | 786 | N.D. | N.D. | |
| DQN0139bb | 73.5 | 60.5 | 71.9 | 248 | 119 | N.D. | 75.9 | 76.5 | 76.9 | |
| DQN0344xx//DQN0385ee | 19.0 | 0.606 | 17.7 | 3.23 | 46.9 | N.D. | 33.1 | 59.4 | 892 | |
| DQN0344xx//DQN0429cc | 49.8 | 1.96 | 39.7 | 14.6 | 94.7 | N.D. | 415 | 171 | N.D. | |

The human IgG4-derived delta-GK Fc fragment was expressed using the FreeStyle™ 293 expression system (Invitrogen). The expressed Fc fragment was purified from the harvested cell culture media by affinity chromatography (MabSelect SuRe, GE). In the final step, we exchanged the buffer to D-PBS(−).

### Reference Example 2

Generation of Anti-Delta-GK Antibody

Anti-delta-GK antibodies were prepared, selected, and assayed as described below.

NZW rabbits were immunized intradermally with the human IgG4-derived delta-GK Fc fragment expressed in Reference Example 1 (100-200 micro g/dose/head). The dose was repeatedly given 6 times over a 3-month period followed by blood and spleen collection. For B-cell selection, an IgG4 delta-GK antibody (an IgG4 antibody with genetically deleted IgG4 C-terminal GK) and a wild type IgG4 antibody were prepared. Delta-GK specific B-cells were sorted using a cell sorter and then plated and cultured according to the procedure described in WO2016098356A1. After cultivation, the B-cell culture supernatants were harvested for further analysis and the corresponding B-cell pellets were cryopreserved.

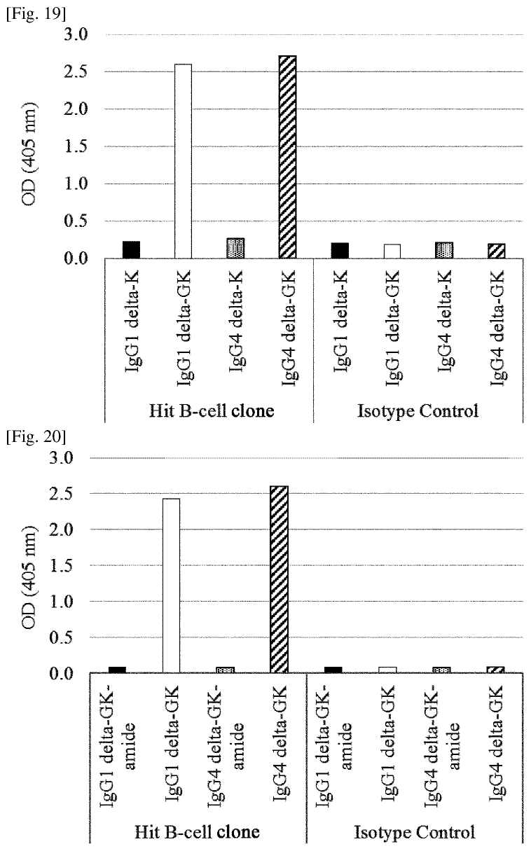

Specific binding to IgG delta-GK was evaluated by ELISA using the B-cell culture supernatants. In this primary screening, four types of antibodies were used as antigens in order to evaluate the binding specificity against the delta-GK C-terminal sequence: an IgG1 antibody with genetically deleted IgG1 C-terminal K (IgG1 delta-K), an IgG1 antibody with genetically deleted IgG1 C-terminal GK (IgG1 delta-GK), an IgG4 antibody with genetically deleted IgG4 C-terminal K (IgG4 delta-K) and an IgG4 antibody with genetically deleted IgG4 C-terminal GK (IgG4 delta-GK). The results showed that only one culture supernatant sample from a single B cell clone demonstrated specific binding to both IgG1 delta-GK and IgG4 delta-GK (FIG. 19).

Delta-GK Fc is more structurally similar to delta-GK-amide Fc than to delta-K Fc. We have also characterized specific binding to delta-GK Fc and delta-GK-amide Fc using the aforementioned selected culture supernatant from the positive B cell clone. IgG1 delta-GK-amide and IgG4 delta-GK-amide were prepared by PAM treatment with IgG1 delta-K or IgG4 delta-K mentioned above and were purified by conventional method. In this secondary screening, four types of antibodies were used as antigens in an ELISA assay to evaluate the binding specificity against the delta-GK C-terminal sequence: IgG1 delta-GK, IgG1 delta-GK-amide, IgG4 delta-GK, and IgG4 delta-GK-amide. Surprisingly, the tested single B cell culture supernatant showed extremely high specificity against delta-GK molecules (FIG. 20).

Based on these screening results, the RNA of the selected clone was extracted from its cryopreserved cell pellet using ZR-96 Quick-RNA kits (ZYMO RESEARCH, Cat No. R1053). DNA encoding the antibody heavy chain variable region in the antibody produced by the selected clone was obtained and amplified by reverse transcription PCR then recombined with DNA encoding the rabbit IgG heavy chain constant region (SEQ ID NO: 75). DNA encoding the antibody light chain variable regions was also obtained and amplified by reverse transcription PCR then recombined with DNA encoding the rabbit Igk light chain constant region (SEQ ID NO: 76). An anti-delta-GK antibody, termed “YG55”, which has two heavy chains and two light chains, was produced from these recombinants. The VH, VL and HVRs sequences of the heavy and light chains are described below. YG55 was expressed using the FreeStyle™ 293 expression system and purified from the culture supernatants.

### Reference Example 3

Characterization of Anti-Delta-GK Monoclonal Antibody YG55

After gene cloning and antibody expression, the specificity of YG55 was assessed by the ELISA assay as described above in the secondary screening. The antibody gene cloning was successful, resulting in YG55 retaining the same specificity shown by the hit (positive) B-cell clone (FIG. 21). This highly specific binding was also confirmed by surface plasmon resonance assay. The specific binding motif and its epitope were identified by crystal structure analysis.

TABLE 8

| | SEQ ID NO: | | | | | | | |
|---|---|---|---|---|---|---|---|---|
| Antibody | | HVR-H1 | HVR-H2 | HVR-H3 | | HVR-Ll | HVR-L2 | HVR-L3 |
| Name | VH | (HCDR1) | (HCDR2) | (HCDR3) | VL | (LCDR1) | (LCDR2) | (LCDR3) |
| YG55 | 77 | 78 | 79 | 80 | 81 | 82 | 83 | 84 |

1. An antigen-binding molecule which has binding activity to at least one, two, three, four, five, six, seven, eight, nine, or all of: complex formed by HLA-DQ2.5 and a BC hordein peptide; complex formed by HLADQ2.5 and a gamma 1 gliadin peptide; complex formed by HLADQ2.5 and a gamma 2 gliadin peptide; complex formed by HLADQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,
wherein the antigen-binding molecule has substantially no binding activity to either or both of a HLA-DQ2.5 positive PBMC B cell and a Ba/F3 cell that expresses HLA-DQ2.5.

2. The antigen-binding molecule of claim 1, wherein the antigen-binding molecule has binding activity to at least one, two, three, or four or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; and complex formed by HLA-DQ2.5 and a 14 mer 1 peptide,
wherein the antigen-binding molecule has substantially no binding activity to either or both of a HLA-DQ2.5 positive PBMC B cell and a Ba/F3 cell that expresses HLA-DQ2.5.

3. The antigen-binding molecule of claim 1, wherein the antigen-binding molecule has binding activity to at least three, four, five, six, seven, eight, nine, or all of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,
wherein the antigen-binding molecule has substantially no binding activity to either or both of a HLA-DQ2.5 positive PBMC B cell and a Ba/F3 cell that expresses HLA-DQ2.5.

4. An antigen-binding molecule which has binding activity to all of: complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide,
wherein the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLA-DQ2.5.

5. The antigen-binding molecule of claim 4, wherein the antigen-binding molecule has binding activity to all of: complex formed by HLADQ2.5 and a BC hordein peptide; complex formed by HLA-DQ2.5 and a gamma 1 gliadin peptide; complex formed by HLA-DQ2.5 and a gamma 2 gliadin peptide; and complex formed by HLA-DQ2.5 and a 26 mer gliadin,
wherein the antigen-binding molecule has substantially no binding activity to at least one, two, three, four, five, or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; a HLA-DQ2.5 positive PBMC B cell; and a Ba/F3 cell that expresses HLA-DQ2.5.

6. The antigen-binding molecule of claim 5, wherein the antigen-binding molecule has binding activity to a complex formed by HLA-DQ2.5 and an immune dominant peptide related to celiac disease.

7. The antigen-binding molecule of claim 5, wherein the antigen-binding molecule has binding activity to all of: a complex formed by HLADQ2.5 and an immune dominant peptide related to celiac disease; complex formed by HLA-DQ2.5 and a 26 mer gliadin peptide; and complex formed by HLA-DQ2.5 and a 14 mer 1 peptide.

8. The antigen-binding molecule of claim 5, wherein the antigen-binding molecule has binding activity to all of: complex formed by HLADQ2.5 and a 33mer gliadin peptide; complex formed by HLA-DQ2.5 and an alpha 1 gliadin peptide; complex formed by HLA-DQ2.5 and an alpha 2 gliadin peptide; complex formed by HLA-DQ2.5 and a gamma 1 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and an alpha 3 gliadin peptide; complex formed by HLA-DQ2.5 and an alpha 1b gliadin peptide; complex formed by HLA-DQ2.5 and a gamma 4b gliadin peptide; complex formed by HLA-DQ2.5 and an avenin 1 peptide; complex formed by HLA-DQ2.5 and an avenin 2 peptide; complex formed by HLA-DQ2.5 and a hordein 1 peptide; complex formed by HLA-DQ2.5 and a hordein 2 peptide; complex formed by HLA-DQ2.5 and a secalin 1 peptide; complex formed by HLA-DQ2.5 and a secalin 2 peptide; complex formed by HLA-DQ2.5 and a 14mer 1 peptide; and complex formed by HLA-DQ2.5 and a 26mer gliadin peptide.

9. The antigen-binding molecule of any one of claim 1 to 8, wherein the antigen-binding molecule blocks the interaction between HLADQ2.5/gluten peptide complex and HLA-DQ2.5/gluten peptide-restricted CD4+ T cell.

10. The antigen-binding molecule of any one of claim 1 or 9, wherein the antigen-binding molecule has substantially no binding activity to HLADQ8, HLA-DQ2.2, HLA-DQ7.5, HLA-DQ5.1, HLA-DQ6.3, HLADQ7.3, HLA-DR or HLA-DP.

11. The antigen-binding molecule of any one of claim 1 to 10, wherein the antigen-binding molecule has enhanced binding activity to a complex formed by HLA-DQ2.5 and a gluten peptide.

12. The antigen-binding molecule of any one of claim 1 to 11, wherein the antigen-binding molecule has stronger binding activity to at least one, two, three, four, five, six, seven, eight, nine, or all of: complex formed by HLA-DQ2.5 and a BC hordein peptide; complex formed by HLADQ2.5 and a gamma 1 gliadin peptide; complex formed by HLADQ2.5 and a gamma 2 gliadin peptide; complex formed by HLADQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,
compared to at least one, two, three, four, five or all of: complex formed by HLA-DQ2.5 and a CLIP peptide; complex formed by HLADQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

13. An antigen-binding molecule which has binding activity to at least one, two, three, four, five, six, seven, eight, nine, or all of: complex formed by HLA-DQ2.5 and a BC hordein peptide; complex formed by HLADQ2.5 and a gamma 1 gliadin peptide; complex formed by HLADQ2.5 and a gamma 2 gliadin peptide; complex formed by HLADQ2.5 and a 26 mer gliadin peptide; complex formed by HLA-DQ2.5 and a 14 mer 1 peptide; complex formed by HLA-DQ2.5 and a 33mer gliadin peptide; complex formed by HLA-DQ2.5 and an omega 2 gliadin peptide; complex formed by HLA-DQ2.5 and an alpha 1 gliadin peptide; complex formed by HLA-DQ2.5 and an alpha 2 gliadin peptide; and complex formed by HLA-DQ2.5 and an omega 1 gliadin peptide,
wherein the antigen-binding molecule has substantially no binding activity to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and an HLADQ2.5 positive PBMC B cell,
wherein the antigen-binding molecule blocks the interaction between HLA-DQ2.5/gluten peptide complex and HLA-DQ2.5/gluten peptide-restricted CD4+ T cell.

14. The antigen-binding molecule of any one of claims 1 to 13, which is any one of (1) to (5) below:
(1) an antigen-binding molecule comprising the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2
sequence of SEQ ID NO: 19, and the LCDR3 sequence of SEQ ID NO: 20;
(2) an antigen-binding molecule comprising the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;
(3) an antigen-binding molecule comprising the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28;
(4) an antigen-binding molecule that binds to the same epitope bound by the antigen-binding molecule of any one of (1) to (3);
(5) an antigen-binding molecule that competes with the antigen-binding molecule of any one of (1) to (3) for binding to HLA-DQ2.5 or a complex formed by HLA-DQ2.5 and a gluten peptide.

15. The antigen-binding molecule of any one of claims 1 to 14, wherein the antigen-binding molecule is a bispecific antigen-binding molecule.

16. The antigen-binding molecule of claim 15, wherein the bispecific antigen-binding molecule is a bispecific antibody.

17. An antigen-binding molecule that comprises at least two antigen-binding domains, wherein either of the antigen-binding domains has binding activity to one or more complexes formed between HLADQ2.5 and an immune dominant peptide related to celiac disease,
wherein either of the antigen-binding domains has substantially no binding activity to at least one, two, three, four, five or all of: complex formed by HLA-DQ2.5 and CLIP peptide; complex formed by HLADQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

18. An antigen-binding molecule that comprises at least two antigen-binding domains, wherein either of the antigen-binding domains has binding activity to all of: complex formed by HLA-DQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; and complex formed by HLA-DQ2.5 and a BC hordein peptide,
wherein either of the antigen-binding domains has substantially no binding activity to at least one, two, three, four, five or all of: complex formed by HLA-DQ2.5 and CLIP peptide; complex formed by HLADQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

19. The antigen-binding molecule of claim 18, wherein either of the antigen-binding domains has binding activity to all of: complex formed by HLA-DQ2.5 and an alpha 1 gliadin peptide; complex formed by HLA-DQ2.5 and an alpha 2 gliadin peptide; complex formed by HLADQ2.5 and an omega 1 gliadin peptide; complex formed by HLADQ2.5 and an omega 2 gliadin peptide; complex formed by HLADQ2.5 and a BC hordein peptide; complex formed by HLA-DQ2.5 and a gamma 1 gliadin peptide; and complex formed by HLA-DQ2.5 and a gamma 2 gliadin peptide.

20. The antigen-binding molecule of claim 19, wherein either of the antigen-binding domains has binding activity to all of: complex formed by HLA-DQ2.5 and an alpha 1 gliadin peptide; complex formed by HLA-DQ2.5 and an alpha 2 gliadin peptide; complex formed by HLADQ2.5 and an omega 1 gliadin peptide; complex formed by HLADQ2.5 and an omega 2 gliadin peptide; complex formed by HLADQ2.5 and a BC hordein peptide; and complex formed by HLA-DQ2.5 and a gamma 1 gliadin peptide.

21. An antigen-binding molecule that comprises at least two antigen-binding domains, wherein either of the antigen-binding domains has binding activity to all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLADQ2.5 and a gamma 2 gliadin peptide; complex formed by HLADQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLADQ2.5 and a gamma 4b gliadin peptide,
wherein either of the antigen-binding domains has substantially no binding activity to at least one, two, three, four, five or all of: complex formed by HLA-DQ2.5 and CLIP peptide; complex formed by HLADQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

22. The antigen-binding molecule of claim 21, wherein either of the antigen-binding domains has binding activity to all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLADQ2.5 and an omega 1 gliadin peptide; complex formed by HLADQ2.5 and an omega 2 gliadin peptide; complex formed by HLADQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide.

23. An antigen-binding molecule that comprises a first antigen-binding domain and a second antigen-binding domain, wherein the first antigen-binding domain has binding activity to one or more complexes formed by HLA-DQ2.5 and a gluten peptide, wherein the second antigen-binding domain has binding activity to one or more complexes formed by HLA-DQ2.5 and a gluten peptide, wherein at least one gluten peptide in the complexes bound by the first antigen-binding domain is different from at least one gluten peptide in the complexes bound by the second antigen-binding domain.

24. The antigen-binding molecule of claim 23, wherein the antigen-binding molecule has binding activity to all of: complex formed by HLADQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide.

25. The antigen-binding molecule of claim 23, wherein the antigen-binding molecule has binding activity to all of: complex formed by HLADQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; and complex formed by HLA-DQ2.5 and a gamma 1 gliadin peptide.

26. The antigen-binding molecule of claim 23, wherein the antigen-binding molecule has binding activity to all of: complex formed by HLADQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; complex formed by HLA-DQ2.5 and a gamma 1 gliadin peptide; and complex formed by HLA-DQ2.5 and a gamma 2 gliadin peptide,
wherein the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

27. The antigen-binding molecule of claim 26, wherein the antigen-binding molecule has binding activity to all of: complex formed by HLADQ2.5 and an alpha 1 gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a BC hordein peptide; and complex formed by HLA-DQ2.5 and a gamma 1 gliadin peptide.

28. An antigen-binding molecule that comprises a first antigen-binding domain which has binding activity to a complex formed by HLADQ2.5 and a first gluten peptide, and a second antigen-binding domain which has binding activity to a complex formed by HLA-DQ2.5 and a second gluten peptide,
wherein the antigen-binding molecule has binding activity to at least two or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLADQ2.5 and a gamma 2 gliadin peptide; complex formed by HLADQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLADQ2.5 and a gamma 4b gliadin peptide,
wherein the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

29. The antigen-binding molecule of claim 28, wherein the antigen-binding molecule has binding activity to at least two or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLA-DQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide,

30. An antigen-binding molecule that comprises a first antigen-binding domain and a second antigen-binding domain,
wherein the first antigen-binding domain has binding activity to at least one or more of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; and complex formed by HLA-DQ2.5 and a 33 mer gliadin peptide;
wherein the second antigen-binding domain has binding activity to at least one or more of: complex formed by HLA-DQ2.5 and a BC hordein peptide; complex formed by HLA-DQ2.5 and a gamma 1 gliadin peptide; complex formed by HLA-DQ2.5 and a gamma 2 gliadin peptide; complex formed by HLA-DQ2.5 and a 26mer gliadin peptide; complex formed by HLA-DQ2.5 and a 14mer 1 peptide; complex formed by HLA-DQ2.5 and a 33 mer gliadin peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide,
wherein the antigen-binding molecule has substantially no binding activity to at least one, two, three, four, five or all of: complex formed by HLA-DQ2.5 and CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell,
wherein the antigen-binding molecule is a bispecific or multispecific antigen-binding molecule.

31. The antigen-binding molecule of claim 30, wherein the second antigen-binding domain has binding activity to at least one or more of: complex formed by HLA-DQ2.5 and a BC hordein peptide; complex formed by HLA-DQ2.5 and a gamma 1 gliadin peptide; complex formed by HLADQ2.5 and a 26mer gliadin peptide; complex formed by HLA-DQ2.5 and a 14mer 1 peptide; complex formed by HLA-DQ2.5 and a 33 mer gliadin peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLA-DQ2.5 and a gamma 4b gliadin peptide.

32. The antigen-binding molecule of any one of claims 17 to 31, wherein the antigen-binding molecule blocks the interaction between HLADQ2.5/gluten peptide complex and HLADQ2.5/gluten peptide-restricted CD4+ T cell.

33. The antigen-binding molecule of any one of claim 17 to 32, wherein the antigen-binding molecule has substantially no binding activity to HLADQ2.2, HLA-DQ7.5, HLA-DQ5.1, HLA-DQ6.3, HLADQ7.3, HLA-DR, or HLA-DP.

34. The antigen-binding molecule of any one of claims 17 to 33, which has enhanced binding activity to a complex formed by HLA-DQ2.5 and a gluten peptide.

35. The antigen-binding molecule of any one of claims 17 to 34, wherein the antigen-binding molecule has stronger binding activity to at least two, three, four, five, six, seven, eight, nine, ten, eleven, twelve, 13, 14, 15, 16, 17, 18, or all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLADQ2.5 and a gamma 2 gliadin peptide; complex formed by HLADQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLADQ2.5 and a gamma 4b gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

36. The antigen-binding molecule of any one of claims 17 to 35, wherein the antigen-binding molecule has stronger binding activity to at least two, three, four, five, six, seven, eight, nine, ten, eleven, twelve, 13, 14, 15, 16, 17, or all of: complex formed by HLA-DQ2.5 and an alpha 1 gliadin peptide; complex formed by HLA-DQ2.5 and an alpha 1b gliadin peptide; complex formed by HLA-DQ2.5 and an alpha 2 gliadin peptide; complex formed by HLA-DQ2.5 and an omega 1 gliadin peptide; complex formed by HLA-DQ2.5 and an omega 2 gliadin peptide; complex formed by HLA-DQ2.5 and a secalin 1 peptide; complex formed by HLA-DQ2.5 and a secalin 2 peptide; complex formed by HLA-DQ2.5 and a BC hordein peptide; complex formed by HLA-DQ2.5 and a gamma 1 gliadin peptide; complex formed by HLADQ2.5 and a 26mer gliadin peptide; complex formed by HLA-DQ2.5 and a 14mer 1 peptide; complex formed by HLA-DQ2.5 and an alpha 3 gliadin peptide; complex formed by HLA-DQ2.5 and an avenin 1 peptide; complex formed by HLA-DQ2.5 and an avenin 2 peptide; complex formed by HLA-DQ2.5 and an avenin 3 peptide; complex formed by HLA-DQ2.5 and a hordein 1 peptide; complex formed by HLA-DQ2.5 and a hordein 2 peptide; and complex formed by HLADQ2.5 and a gamma 4b gliadin peptide, compared to at least one, two, three, four, five or all of: complex formed by HLA-DQ2.5 and a CLIP peptide; complex formed by HLA-DQ2.5 and a salmonella peptide; complex formed by HLA-DQ2.5 and a Mycobacterium bovis peptide; complex formed by HLA-DQ2.5 and a Hepatitis B virus peptide; complex formed by HLA-DQ2.5 and a thyroperoxidase peptide; and a HLA-DQ2.5 positive PBMC B cell.

37. The antigen-binding molecule of any one of claims 17 to 36, which is any one of (1) to (5) below:
(1) an antigen-binding molecule comprising the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2
sequence of SEQ ID NO: 19, and the LCDR3 sequence of SEQ ID NO: 20;
(2) an antigen-binding molecule comprising the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;
(3) an antigen-binding molecule comprising the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28;
(4) an antigen-binding molecule that binds to the same epitope bound by the antigen-binding molecule of any one of (1) to (3);
(5) an antigen-binding molecule that competes with the antigen-binding molecule of any one of (1) to (3) for binding to HLA-DQ2.5 or a complex formed by HLA-DQ2.5 and a gluten peptide.

38. The antigen-binding molecule of any one of claims 17 to 37, wherein the antigen-binding molecule is a bispecific antigen-binding molecule.

39. The antigen-binding molecule of claim 38, wherein the bispecific antigen-binding molecule is a bispecific antibody.

40. The antigen-binding molecule of any one of claims 37 to 39, which is any one of (a) to (d) below:
(a) an antigen-binding molecule comprising (i) and (iii) below,
(b) an antigen-binding molecule comprising (ii) and (iii) below,
(c) an antigen-binding molecule that binds to the same epitope bound by the antigen-binding molecule of (a) or (b),
(d) an antigen-binding molecule that competes with the antigen-binding molecule of (a) or (b) for binding to HLA-DQ2.5 or a complex formed by HLA-DQ2.5 and a gluten peptide,
(i) the HCDR1 sequence of SEQ ID NO: 2, the HCDR2 sequence of SEQ ID NO: 3, the HCDR3 sequence of SEQ ID NO: 4, the LCDR1 sequence of SEQ ID NO: 18, the LCDR2 sequence of SEQ ID NO: 19, and the LCDR3 sequence of SEQ ID NO: 20;
(ii) the HCDR1 sequence of SEQ ID NO: 6, the HCDR2 sequence of SEQ ID NO: 7, the HCDR3 sequence of SEQ ID NO: 8, the LCDR1 sequence of SEQ ID NO: 22, the LCDR2 sequence of SEQ ID NO: 23, and the LCDR3 sequence of SEQ ID NO: 24;
(iii) the HCDR1 sequence of SEQ ID NO: 10, the HCDR2 sequence of SEQ ID NO: 11, the HCDR3 sequence of SEQ ID NO: 12, the LCDR1 sequence of SEQ ID NO: 26, the LCDR2 sequence of SEQ ID NO: 27, and the LCDR3 sequence of SEQ ID NO: 28.

41. The antigen-binding molecule of any one of claims 9, 13, and 32,
wherein the gluten peptide(s) is/are one, two, three, four, five, six, seven, eight, or all of alpha 1 gliadin peptide, alpha 2 gliadin peptide, omega 1 gliadin peptide, omega 2 gliadin peptide, gamma 1 gliadin peptide, gamma 2 gliadin peptide, BC hordein peptide, alpha 1b gliadin peptide, and gamma 4a gliadin peptide.

42. The antigen-binding molecule of claim 41, wherein the gluten peptides are alpha 1 gliadin peptide, alpha 2 glaidin peptide, omega 1 gliadin peptide, and alpha 1b gliadin peptide.

43. The antigen-binding molecule of claim 41, wherein the gluten peptides are alpha 2 glaidin peptide, omega 2 gliadin peptide, gamma 1 gliadin peptide, gamma 2 gliadin peptide, BC hordein peptide, alpha 1b glaidin peptide, and gamma 4a gliadin peptide.

44. The antigen-binding molecule of claim 41, wherein the gluten peptides are alpha 2 glaidin peptide, omega 2 gliadin peptide, gamma 1 gliadin peptide, and BC hordein peptide.

45. The antigen-binding molecule of claim 41, wherein the gluten peptides are alpha 1 gliadin peptide, alpha 2 glaidin peptide, omega 1 gliadin peptide, omega 2 gliadin peptide, gamma 1 gliadin peptide, BC hordein peptide, alpha 1b glaidin peptide, gamma 4a gliadin peptide, and gamma 2 gliadin peptide.

46. The antigen-binding molecule of claim 41, wherein the gluten peptides are alpha 1 gliadin peptide, alpha 2 glaidin peptide, omega 1 gliadin peptide, omega 2 gliadin peptide, gamma 1 gliadin peptide, BC hordein peptide, and alpha 1b glaidin peptide.

47. A nucleic acid encoding the antigen-binding molecule of any one of claims 1 to 46.

48. A vector into which the nucleic acid of claim 47 is introduced.

49. A cell comprising the nucleic acid of claim 47 or the vector of claim 48.

50. A method of producing an antigen-binding molecule by culturing the cell of claim 49.


SEQUENCE LISTING (97 sequences)

SEQ ID NO: 1 - protein, 117 aa, Oryctolagus cuniculus
QEQLEESGGDLVKPGASLTLTCTASGFSFYSSYWICWVRQAPGKGLEWIACIDTGSGSIDYASWVNGRFTISKTSSTTVTLQMTSLTVADTATYFCARDIGIDYNFWGPGTLVTVSS

SEQ ID NO: 2 - protein, 6 aa, Artificial Sequence
  DQN0385He HCDR1
SSYWIC

SEQ ID NO: 3 - protein, 17 aa, Artificial Sequence
  DQN0385He HCDR2
CIDTGSGSIDYASWVNG

SEQ ID NO: 4 - protein, 9 aa, Artificial Sequence
  DQN0385He HCDR3
DIGIDYNFW

SEQ ID NO: 5 - protein, 117 aa, Oryctolagus cuniculus
QEQLVESGGGLVQPEGSLTLTCTASGFSFSSSYYMCWVRQAPGKGLEWIGCIYTGSGSTDYATWVNGRFTISKTSSTTVTLQMTSLTVADTATYFCARDIGIDYNFWGPGTLVTVSS

SEQ ID NO: 6 - protein, 6 aa, Artificial Sequence
  DQN0429Hc HCDR1
SSYYMC

SEQ ID NO: 7 - protein, 17 aa, Artificial Sequence
  DQN0429Hc HCDR2
CIYTGSGSTDYATWVNG

SEQ ID NO: 8 - protein, 9 aa, Artificial Sequence
  DQN0429Hc HCDR3
DIGIDYNFW

SEQ ID NO: 9 - protein, 121 aa, Oryctolagus cuniculus
QSLEESGGGLVKPGGTLTLTCTASGFSFSSSYWMCWVRQAPGKGLEWVACVYGGSDTTYYASWTKGRFTISKGSSTTVALQVTSLTAADTATYFCARDPLNYYYYGELNLWGPGTLVTVSS

SEQ ID NO: 10 - protein, 6 aa, Artificial Sequence
  DQN0344Hx HCDR1
SSYWMC

SEQ ID NO: 11 - protein, 17 aa, Artificial Sequence
  DQN0344Hx HCDR2
CVYGGSDTTYYASWTKG

SEQ ID NO: 12 - protein, 13 aa, Artificial Sequence
  DQN0344Hx HCDR3
DPLNYYYYGELNL

SEQ ID NO: 13 - protein, 116 aa, Artificial Sequence
  IC17 VH
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 14 - protein, 5 aa, Artificial Sequence
  IC17 HCDR1
SYWMH

SEQ ID NO: 15 - protein, 17 aa, Artificial Sequence
  IC17 HCDR2
MIDPSYSETRLNQKFKD

SEQ ID NO: 16 - protein, 7 aa, Artificial Sequence
  IC17 HCDR3
YGNYFDY

SEQ ID NO: 17 - protein, 106 aa, Oryctolagus cuniculus
AFELTQTPSFVEAAVGGTVTIKCQATQSISSYLNWYQQKPGQPPKLLIYYASTLASGVPSRFKGSGSGTEFSLTISGVECADAATYYCHYGISYVSFGGGTKVEIK

SEQ ID NO: 18 - protein, 11 aa, Artificial Sequence
  DQN0385Le LCDR1
QATQSISSYLN

SEQ ID NO: 19 - protein, 7 aa, Artificial Sequence
  DQN0385Le LCDR2
YASTLAS

SEQ ID NO: 20 - protein, 8 aa, Artificial Sequence
  DQN0385Le LCDR3
HYGISYVS

SEQ ID NO: 21 - protein, 106 aa, Oryctolagus cuniculus
AFELTQTPSFVEARVGGTVTIKCQATQSISSYLNWYQQKPGQPPKLLIYYASTLASGVPSRFKGSGSGTEFTLTISGVECADAATYYCHYGISYVSFGGGTKVEIK

SEQ ID NO: 22 - protein, 11 aa, Artificial Sequence
  DQN0429Lc LCDR1
QATQSISSYLN

SEQ ID NO: 23 - protein, 7 aa, Artificial Sequence
  DQN0429Lc LCDR2
YASTLAS

SEQ ID NO: 24 - protein, 8 aa, Artificial Sequence
  DQN0429Lc LCDR3
HYGISYVS

SEQ ID NO: 25 - protein, 108 aa, Oryctolagus cuniculus
AVVLTQTASPVSAAVGGTVTIRCQATENIYSGLAWYQQKPGQPPKVLIYYVSTLASGVPSRFKGSGSGTEYTLTISGVQCDDAATYYCQTYHDISNVTFGGGTKVEIK

SEQ ID NO: 26 - protein, 11 aa, Artificial Sequence
  DQN0344Lx LCDR1
QATENIYSGLA

SEQ ID NO: 27 - protein, 7 aa, Artificial Sequence
  DQN0344Lx LCDR2
YVSTLAS

SEQ ID NO: 28 - protein, 10 aa, Artificial Sequence
  DQN0344Lx LCDR3
QTYHDISNVT

SEQ ID NO: 29 - protein, 107 aa, Artificial Sequence
  IC17 VL
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVK

SEQ ID NO: 30 - protein, 11 aa, Artificial Sequence
  IC17 LCDR1
KASEDIYNRLA

SEQ ID NO: 31 - protein, 7 aa, Artificial Sequence
  IC17 LCDR2
GATSLET

SEQ ID NO: 32 - protein, 9 aa, Artificial Sequence
  IC17 LCDR3
QQYWSTPYT

SEQ ID NO: 33 - protein, 328 aa, Artificial Sequence
  SG18 S3n CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 34 - protein, 328 aa, Artificial Sequence
  SG18 S3p CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 35 - protein, 107 aa, Artificial Sequence
  k0mc CL
RTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 36 - protein, 107 aa, Artificial Sequence [sequence omitted]

SEQ ID NO: 37 - protein, 19 aa, Artificial Sequence
  CAMPATH-1H signal sequence
MGWSCIILFLVATATGVHS

SEQ ID NO: 38 - protein, 4 aa, Artificial Sequence
  GGGG linker
GGGG

SEQ ID NO: 39 - protein, 33 aa, Artificial Sequence
  33-mer gliadin peptide
LQLQPFPQPELPYPQPELPYPQPELPYPQPQPF

SEQ ID NO: 40 - protein, 5 aa, Artificial Sequence
  GGGGG linker
GGGGG

SEQ ID NO: 41 - protein, 12 aa, Artificial Sequence
  SSADLVPRGGGG linker
SSADLVPRGGGG

SEQ ID NO: 42 - protein, 18 aa, Artificial Sequence
  gliadin peptide
QQYPSGEGSFQPSQENPQ

SEQ ID NO: 43 - protein, 13 aa, Artificial Sequence
  SSADLVPRGGGGG linker
SSADLVPRGGGGG

SEQ ID NO: 44 - protein, 20 aa, Artificial Sequence
  DBY peptide
ATGSNCPPHIENFSDIDMGE

SEQ ID NO: 45 - protein, 29 aa, Artificial Sequence
  CLIP peptide
KLPKPPKPVSKMRMATPLLMQALPMGALP

SEQ ID NO: 46 - protein, 8 aa, Artificial Sequence
  3C protease cleavage linker
LEVLFQGP

SEQ ID NO: 47 - protein, 11 aa, Artificial Sequence
  gamma 2 gliadin peptide
IIQPEQPAQLP

SEQ ID NO: 48 - protein, 17 aa, Artificial Sequence
  BC Hordein peptide
EPEQPIPEQPQPYPQQP

SEQ ID NO: 49 - DNA, 945 bp, Homo sapiens
gaattccaccatgggctggtcctgcatcatcctgttcctggtggcaaccgcaacaggagtgcactctggcgtgtcccagtctcccagcaacaaggtgaccgagaagggcaaggacgtggagctgcggtgtgatcctatcagcggccacacagccctgtactggtatagacagtccctgggccagggcctggagttcctgatctactttcagggcaatagcgcccctgacaagtctggcctgccaagcgatcggttctccgccgagagaaccggaggatccgtgtctaccctgacaatccagcggacacagcaggaggactccgccgtgtacctgtgcgccagctccttcaggtttaccgatacacagtattttggaccaggaaccaggctgacagtgctggaggacctgaagaacgtgttcccccctgaggtggccgtgtttgagccatctgaggccgagatcagccacacccagaaggccacactggtgtgcctggccaccggcttctaccccgatcacgtggagctgtcctggtgggtgaacggcaaggaggtgcactctggcgtgtgcacagacccacagcccctgaaggagcagcctgccctgaatgattctcggtacgccctgtctagccggctgagagtgagcgccaccttttggcagaacccaaggaatcacttccgctgccaggtgcagttttatggcctgagcgagaatgacgagtggacccaggatagggccaagcctgtgacacagatcgtgtccgccgaggcatggggaagggcagactgtggcttcaccagcgtgtcctatcagcagggcgtgctgtctgccaccatcctgtacgagatcctgctgggcaaggccacactgtatgccgtgctggtgtccgccctggtgctgatggccatggtgaagagaaaggatttttagtaagcggccgc

SEQ ID NO: 50 - protein, 15 aa, Artificial Sequence
  Hepatits B virus peptide
PDRVHFASPLHVAWR

SEQ ID NO: 51 - protein, 9 aa, Artificial Sequence
  Salmonella peptide
MMAWRMMRY

SEQ ID NO: 52 - protein, 16 aa, Artificial Sequence
  Thyroperoxidase peptide
YIDVWLGGLAENFLPY

SEQ ID NO: 53 - protein, 14 aa, Artificial Sequence
  Mycobacterium bovis peptide
KPLLIIAEDVEGEY

SEQ ID NO: 54 - protein, 11 aa, Artificial Sequence
  alpha 1 gliadin peptide
QPFPQPELPYP

SEQ ID NO: 55 - protein, 11 aa, Artificial Sequence
  alpha 2 gliadin peptide
FPQPELPYPQP

SEQ ID NO: 56 - protein, 11 aa, Artificial Sequence
  gamma 1 gliadin peptide
QPQQSFPEQQQ

SEQ ID NO: 57 - protein, 12 aa, Artificial Sequence
  gamma 2 gliadin peptide
GIIQPEQPAQLP

SEQ ID NO: 58 - protein, 11 aa, Artificial Sequence
  omega 1 gliadin peptide
QPFPQPEQPFP

SEQ ID NO: 59 - protein, 11 aa, Artificial Sequence
  omega 2 gliadin peptide
FPQPEQPFPWQ

SEQ ID NO: 60 - protein, 16 aa, Artificial Sequence
  BC hordein peptide
PQQPIPEQPQPYPQQP

SEQ ID NO: 61 - protein, 11 aa, Artificial Sequence
  alpha 3 gliadin peptide
PFRPEQPYPQP

SEQ ID NO: 62 - protein, 11 aa, Artificial Sequence
  alpha 1b gliadin peptide
LPYPQPELPYP

SEQ ID NO: 63 - protein, 11 aa, Artificial Sequence
  gamma 4b gliadin peptide
FPQPEQEFPQP

SEQ ID NO: 64 - protein, 11 aa, Artificial Sequence
  avenin 1 peptide
QPYPEQEEPFV

SEQ ID NO: 65 - protein, 11 aa, Artificial Sequence
  avenin 2 peptide
QPYPEQEQPFV

SEQ ID NO: 66 - protein, 11 aa, Artificial Sequence
  avenin 3 peptide
QPYPEQEQPIL

SEQ ID NO: 67 - protein, 14 aa, Artificial Sequence
  hordein 1 peptide
PQQPFPQPEQPFRQ

SEQ ID NO: 68 - protein, 14 aa, Artificial Sequence
  hordein 2 peptide
QEFPQPEQPFPQQP

SEQ ID NO: 69 - protein, 14 aa, Artificial Sequence
  secalin 1 peptide
PEQPFPQPEQPFPQ

SEQ ID NO: 70 - protein, 14 aa, Artificial Sequence
  secalin 2 peptide
QPFPQPEQPFPQSQ

SEQ ID NO: 71 - protein, 15 aa, Artificial Sequence
  14mer 1 peptide
PQQQTLQPEQPAQLP

SEQ ID NO: 72 - protein, 26 aa, Artificial Sequence
  26mer gliadin peptide
FLQPEQPFPEQPEQPYPEQPEQPFPQ

SEQ ID NO: 73 - protein, 328 aa, Artificial Sequence
  F1332m CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 74 - protein, 107 aa, Artificial Sequence; identical to SEQ ID NO: 35
  hk0MC CL

SEQ ID NO: 75 - protein, 323 aa, Oryctolagus cuniculus
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 76 - protein, 104 aa, Oryctolagus cuniculus
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 77 - protein, 114 aa, Oryctolagus cuniculus
QSLEESGGRLVTPGGSLTLTCTVSGIDLSNYAVGWVRQAPGKGLEYIGIIYASGSAYYASWAKGRFTISKTSSTTVDLKVTSLTTEDTATYFCARGYGRAFRIWGPGTLVTVSS

SEQ ID NO: 78 - protein, 5 aa, Oryctolagus cuniculus
NYAVG

SEQ ID NO: 79 - protein, 16 aa, Oryctolagus cuniculus
IIYASGSAYYASWAKG

SEQ ID NO: 80 - protein, 8 aa, Oryctolagus cuniculus
GYGRAFRI

SEQ ID NO: 81 - protein, 109 aa, Oryctolagus cuniculus
AVLTQTPSPVSAAVGGTVTISCQASQSVYNNNFLSWYQQKPGQRPKLLIYDASTLESGVPSRFSGSGSGTQFTLTISSVQCDDAATYYCLGGYDDHDNAFGGGTEVVVK

SEQ ID NO: 82 - protein, 13 aa, Oryctolagus cuniculus
QASQSVYNNNFLS

SEQ ID NO: 83 - protein, 7 aa, Oryctolagus cuniculus
DASTLES

SEQ ID NO: 84 - protein, 10 aa, Oryctolagus cuniculus
LGGYDDHDNA

SEQ ID NO: 85 - protein, 117 aa, Oryctolagus cuniculus
QSLEESGGRLVTPGTALTLTCTVSGFSLSSYAMGWVRQAPGKGLEYIGWTSPGDSAYYASWTKGRFTISRTSTTVELKITSPTTEDTATYFCARDAAYIGHWAFNLWGPGTLVTVSS

SEQ ID NO: 86 - protein, 5 aa, Artificial Sequence
  DQN0139Hb HCDR1
SYAMG

SEQ ID NO: 87 - protein, 16 aa, Artificial Sequence
  DQN0139Hb HCDR2
WTSPGDSAYYASWTKG

SEQ ID NO: 88 - protein, 12 aa, Artificial Sequence
  DQN0139Hb HCDR3
DAAYIGHWAFNL

SEQ ID NO: 89 - protein, 110 aa, Oryctolagus cuniculus
DVVMTQTPASVSAPVGGTVTINCQASESIYSNLAWYQQKPGQPPKLLIYGASTLESGVPSRVKGSGYGTQFTLTISDLECADAATYYCQQYYGSSSTAFTFGGGTKVEIK

SEQ ID NO: 90 - protein, 11 aa, Artificial Sequence
  DQN0139Lb LCDR1
QASESIYSNLA

SEQ ID NO: 91 - protein, 7 aa, Artificial Sequence
  DQN0139Lb LCDR2
GASTLES

SEQ ID NO: 92 - protein, 12 aa, Artificial Sequence
  DQN0139Lb LCDR3
QQYYGSSSTAFT

SEQ ID NO: 93 - protein, 22 aa, Artificial Sequence
  2A self-cleaving peptide sequence
GSGATNFSLLKQAGDVEENPGP

SEQ ID NO: 94 - protein, 15 aa, Artificial Sequence
  chemically synthesized omega gliadin W03E7 peptide
EQPFPQPEQPFPWQP

SEQ ID NO: 95 - protein, 16 aa, Artificial Sequence
  chemically synthesized BC hordein B08E2E7 peptide
EPEQPIPEQPQPYPQQ

SEQ ID NO: 96 - protein, 12 aa, Artificial Sequence
  chemically synthesized gamma 4a gliadin peptide
FSQPEQEFPQPQ

SEQ ID NO: 97 - DNA, 825 bp, Homo sapiens
gaattccaccatgggctggagctgcatcatcctgtttctggtggcaaccgcaacaggagtgcacagcaagaccacacagcccccttccatggactgcgcagagggaagggcagcaaacctgccctgtaatcacagcaccatctccggcaacgagtacgtgtattggtacagacagatccactctcagggccctcagtatatcatccacggcctgaagaacaatgagacaaatgagatggcctccctgatcatcaccgaggaccggaagagctccacactgatcctgccacacgccaccctgagagacacagccgtgtactattgcatcgtgctgggaggagcagatggactgacctttggcaagggcacacacctgatcatccagccctatatccagaacccagatcccgccgtgtaccagctgagggactccaagtctagcgataagagcgtgtgcctgttcaccgactttgattctcagacaaacgtgagccagagcaaggacagcgacgtgtacatcaccgacaagacagtgctggatatgagatccatggacttcaagtccaactctgccgtggcctggtctaataagagcgatttcgcctgcgccaacgcctttaacaattccatcatccctgaggataccttctttccttctccagagtcctcttgtgacgtgaagctggtggagaagagcttcgagaccgatacaaacctgaattttcagaacctgtccgtgatcggcttcaggatcctgctgctgaaggtggccggcttcaatctgctgatgaccctgcgcctgtggagctcctagtaagcggccgc